(19) United States
(12) Patent Application Publication
(10) Pub. No.: US 2010/0267743 A1
(43) Pub. Date: Oct. 21, 2010

(54) 3-(PHENOXYPYRROLIDIN-3-YL-METHYL)HETEROARYL, 3-(PHENYLPYRROLIDIN-3-YLMETHOXY)HETEROARYL, AND 3-(HETEROARYLPYRROLIDIN-3-YLMETHOXY)HETEROARYL COMPOUNDS

(76) Inventors: Eric L. Stangeland, Pacifica, CA (US); Priscilla Van Dyke, San Francisco, CA (US); Daisuke Roland Saito, Burlingame, CA (US); Jane Schmidt, San Francisco, CA (US); Lori Jean Patterson, San Francisco, CA (US); Timothy J. Church, San Mateo, CA (US); Adam Hughes, Belmont, CA (US)

Correspondence Address: THERAVANCE, INC., 901 GATEWAY BOULEVARD, SOUTH SAN FRANCISCO, CA, 94080, US

(21) Appl. No.: 12/760,276

(22) Filed: Apr. 14, 2010

Related U.S. Application Data
(60) Provisional application No. 61/169,404, filed on Apr. 15, 2009.

(51) Int. Cl. A61K 31/497; C07D 401/06; A61K 31/4439; C07D 277/28; A61K 31/427; C07D 403/06; A61K 31/506; A61P 25/28; A61P 25/24; A61P 25/08; A61P 25/16; A61P 3/04; A61P 25/02

(52) U.S. Cl. 514/255.05; 546/276.4; 514/343; 548/205; 514/365; 544/336; 514/256; 544/335

(57) ABSTRACT

In one aspect, the invention relates to compounds of formula I:

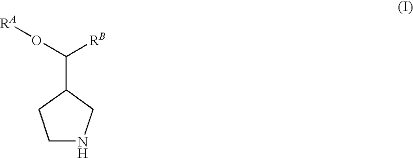

where R^{A} and R^{B} are as defined in the specification, or a pharmaceutically acceptable salt thereof. The compounds of formula I are serotonin and norepinephrine reuptake inhibitors. In another aspect, the invention relates to pharmaceutical compositions comprising such compounds; methods of using such compounds; and process and intermediates for preparing such compounds.

### CROSS-REFERENCE TO RELATED APPLICATIONS

This application claims the benefit of U.S. Provisional Application No. 61/169,404, filed on Apr. 15, 2009; the entire disclosure of which is incorporated herein by reference in its entirety.

### BACKGROUND OF THE INVENTION

1. Field of the Invention

The present invention relates to 3-(phenoxypyrrolidin-3-yl-methyl)heteroaryl, 3-(phenylpyrrolidin-3-ylmethoxy)heteroaryl, and 3-(heteroarylpyrrolidin-3-ylmethoxy)heteroaryl compounds having activity as serotonin (5-HT) and/or norepinephrine (NE) reuptake inhibitors. The invention also relates to pharmaceutical compositions comprising such compounds, processes and intermediates for preparing such compounds and methods of using such compounds to treat a pain disorder, such as neuropathic pain, and other ailments.

2. State of the Art

Pain is an unpleasant sensory and emotional experience associated with actual or potential tissue damage, or described in terms of such damage (International Association for the Study of Pain (IASP), Pain Terminology). Chronic pain persists beyond acute pain or beyond the expected time for an injury to heal (American Pain Society. “Pain Control in the Primary Care Setting.” 2006: 15). Neuropathic pain is pain initiated or caused by a primary lesion or dysfunction in the nervous system. Peripheral neuropathic pain occurs when the lesion or dysfunction affects the peripheral nervous system and central neuropathic pain when the lesion or dysfunction affects the central nervous system (IASP, supra).

Several types of therapeutic agents are currently used to treat neuropathic pain including, for example, tricyclic antidepressants (TCAs), serotonin and norepinephrine reuptake inhibitors (SNRIs), calcium channel ligands (e.g., gabapentin and pregabalin), topical lidocaine, and opioid agonists (e.g., morphine, oxycodone, methadone, levorphanol and tramadol). However, neuropathic pain can be very difficult to treat with no more than 40-60% of patients achieving, at best, partial relief of their pain (Dworkin et al. (2007) Pain 132:237-251). Moreover, all of the therapeutic agents currently used to treat neuropathic pain have various side effects (e.g., nausea, sedation, dizziness and somnolence) that can limit their effectiveness in some patients (Dworkin et al., supra).

SNRIs, such as duloxetine and venlafaxine, are often used as first line therapy for treating neuropathic pain. These agents inhibit the reuptake of both serotonin (5-hydroxytrypamine, 5-HT) and norepinephrine (NE) by binding to the serotonin and norepinephrine transporters (SERT and NET, respectively). However, both duloxetine and venlafaxine have higher affinity for SERT relative to NET (Vaishnavi et al. (2004) Biol. Psychiatry 55(3):320-322).

Preclinical studies suggest that inhibition of both SERT and NET may be necessary for maximally effective treatment of neuropathic and other chronic pain states (Jones et al. (2006) Neuropharmacology 51(7-8):1172-1180; Vickers et al. (2008) Bioorg. Med. Chem. Lett. 18:3230-3235; Fishbain et al. (2000) Pain Med. 1(4):310-316; and Mochizucki (2004) Human Psychopharmacology 19:S15-S19). However, in clinical studies, the inhibition of SERT has been reported to be related to nausea and other side effects (Greist et al. (2004) Clin. Ther. 26(9):1446-1455). Thus, therapeutic agents having more balanced SERT and NET affinity or slightly higher NET affinity are expected to be particularly useful for treating chronic pain while producing fewer side effects, such as nausea.

Thus, a need exists for novel compounds that are useful for treating chronic pain, such as neuropathic pain. In particular, a need exists for novel compounds that are useful for treating chronic pain and that have reduced side effects, such as nausea. A need also exists for novel dual-acting compounds that inhibit both SERT and NET with high affinity (e.g., pKᵢ≧8.0).

### SUMMARY OF THE INVENTION

The present invention provides novel compounds that have been found to possess serotonin reuptake inhibitory activity and/or norepinephrine reuptake inhibitory activity. Accordingly, compounds of the invention are expected to be useful and advantageous as therapeutic agents for those diseases and disorders that can be treated by inhibition of the serotonin and/or norepinephrine transporter, such as neuropathic pain.

One aspect of the invention relates to a compound of formula I:

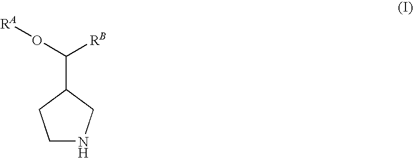

where: R^{A} and R^{B} are independently C₃₋₅heteroaryl, naphthalene, or

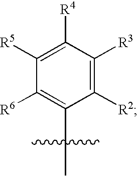

with the proviso that at least one of R^{A} and R^{B} is C₃₋₅heteroaryl or naphthalene; and further with the proviso that when R^{A} is:

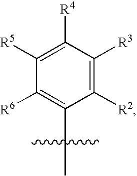

R^{B} is not unsubstituted 2-pyridine; C₃₋₅heteroaryl and naphthalene are optionally substituted with one to four R¹ groups independently selected from halo, —C₁₋₆alkyl, —O—C₁₋₆alkyl, —O—C₃₋₇cycloalkyl, —CN, and —C₀₋₁alkylene-NR^{a}R^{b}; R² through R⁶ are independently selected from H, halo, —C₁₋₆alkyl, —C₂₋₆alkynyl, —O—C₁₋₆alkyl, —C₁₋₄alkylene-O—C₁₋₄alkyl, —C₃₋₇cycloalkyl, —C₀₋₁alkylene-phenyl, —O—C₀₋₃alkylene-phenyl, —C₀₋₆alkylene-OH, —CN, —C₀₋₂alkylene-COOH, —CHO, —C(O)—C₁₋₆alkyl, —C(O)O—C₁₋₄alkyl, —CH₂SH, —S—C₁₋₆alkyl, —C₁₋₄alkylene-S—C₁₋₄alkyl, —SO₂—C₁₋₆alkyl, —SO₂NR^{a}R^{b}, —NHSO₂R^{a}, —C₀₋₁alkylene-NR^{a}R^{b}, —NHC(O)—C₁₋₆alkyl, —C(O)NR^{a}R^{b}, and —NO₂; R^{a} and R^{b} are independently H or —C₁₋₄alkyl; each alkyl in R¹ through R⁶ is optionally substituted with 1 to 5 fluoro atoms; and each phenyl in R² through R⁶ is optionally substituted with 1 or 2 groups independently selected from halo, —C₁₋₆alkyl, and —O—C₁₋₆alkyl; or a pharmaceutically acceptable salt thereof.

Another aspect of the invention relates to compounds of formula I having a configuration selected from:

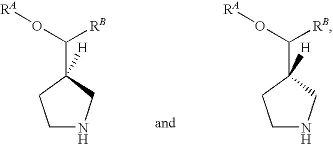

or enriched in a stereoisomeric form having such configuration.

Yet another aspect of the invention relates to compounds of formula I having a configuration selected from:

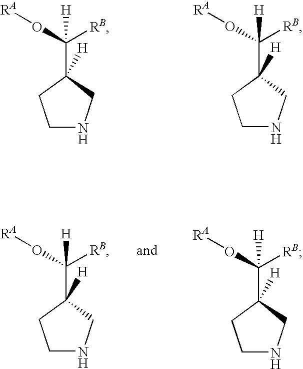

or enriched in a stereoisomeric form having such configuration.

Yet another aspect of the invention relates to pharmaceutical compositions comprising a pharmaceutically acceptable carrier and a compound of the invention. Such compositions may optionally contain other active agents such as anti-Alzheimer's agents, anticonvulsants, antidepressants, anti-Parkinson's agents, dual serotonin-norepinephrine reuptake inhibitors, non-steroidal anti-inflammatory agents, norepinephrine reuptake inhibitors, opioid agonists, selective serotonin reuptake inhibitors, sodium channel blockers, sympatholytics, and combinations thereof. Accordingly, in yet another aspect of the invention, a pharmaceutical composition comprises a compound of the invention, a second active agent, and a pharmaceutically acceptable carrier. Another aspect of the invention relates to a combination of active agents, comprising a compound of the invention and a second active agent. The compound of the invention can be formulated together or separately from the additional agent(s). When formulated separately, a pharmaceutically acceptable carrier may be included with the additional agent(s). Thus, yet another aspect of the invention relates to a combination of pharmaceutical compositions, the combination comprising: a first pharmaceutical composition comprising a compound of formula I or a pharmaceutically acceptable salt thereof and a first pharmaceutically acceptable carrier; and a second pharmaceutical composition comprising a second active agent and a second pharmaceutically acceptable carrier. The invention also relates to a kit containing such pharmaceutical compositions, for example where the first and second pharmaceutical compositions are separate pharmaceutical compositions.

Compounds of the invention possess serotonin reuptake inhibitory activity and norepinephrine reuptake inhibitory activity, and are therefore expected to be useful as therapeutic agents for treating patients suffering from a disease or disorder that is treated by the inhibition of the serotonin and/or the norepinephrine transporter. Thus, one aspect of the invention relates to a method of treating: a pain disorder such as neuropathic pain; a depressive disorder such as major depression; an affective disorder such as an anxiety disorder; attention deficit hyperactivity disorder; a cognitive disorder such as dementia; stress urinary incontinence; obesity; or vasomotor symptoms associated with menopause, comprising administering to a patient a therapeutically effective amount of a compound of the invention.

Still another aspect of the invention relates to a method for inhibiting serotonin reuptake in a mammal comprising administering to the mammal, a serotonin transporter-inhibiting amount of a compound of the invention. Yet another aspect of the invention relates to a method for inhibiting norepinephrine reuptake in a mammal comprising administering to the mammal, a norepinephrine transporter-inhibiting amount of a compound of the invention. And another aspect of the invention relates to a method for inhibiting serotonin reuptake and norepinephrine reuptake in a mammal comprising administering to the mammal, a serotonin transporter- and norepinephrine transporter-inhibiting amount of a compound of the invention.

Among the compounds of formula I, compounds of interest are those having an inhibitory constant (pKᵢ) at SERT or at NET greater than or equal to 5.0, and in some embodiment greater than or equal to 7.0, or greater than or equal to 8.0. Compounds of particular interest are those compounds having a pKᵢ at both SERT and NET greater than or equal to 7.0, and in one embodiment, having a pKᵢ at both SERT and NET greater than or equal to 8.0. In another embodiment, compounds of interest have balanced SERT and NET activity, i.e., have the same pKᵢ value at both SERT and NET±0.5.

Since compounds of the invention possess serotonin reuptake inhibitory activity and norepinephrine reuptake inhibitory activity, such compounds are also useful as research tools. Accordingly, one aspect of the invention relates to methods of using the compounds of the invention as research tools, comprising conducting a biological assay using a compound of the invention. Compounds of the invention can also be used to evaluate new chemical compounds. Thus another aspect of the invention relates to a method of evaluating a test compound in a biological assay, comprising: (a) conducting a biological assay with a test compound to provide a first assay value; (b) conducting the biological assay with a compound of the invention to provide a second assay value; wherein step (a) is conducted either before, after or concurrently with step (b); and (c) comparing the first assay value from step (a) with the second assay value from step (b). Exemplary biological assays include a serotonin reuptake assay and a norepinephrine reuptake assay. Still another aspect of the invention relates to a method of studying a biological system or sample comprising serotonin transporters, norepinephrine transporters, or both, the method comprising: (a) contacting the biological system or sample with a compound of the invention; and (b) determining the effects caused by the compound on the biological system or sample.

The invention also relates to processes and intermediates useful for preparing compounds of the invention. Accordingly, one aspect of the invention relates to a process for preparing a compound of formula I, the process comprising deprotecting a compound of the formula:

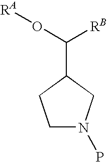

or a salt thereof, where P is an amino-protecting group to provide a compound of formula I, or a salt thereof. In other aspects, the invention relates to novel intermediates used in such processes. In one aspect of the invention, such novel intermediates have the formula of compound 9, 9′ or 9″, as defined herein.

Yet another aspect of the invention relates to the use of compounds of the invention in therapy, and for the manufacture of medicaments, especially for the manufacture of medicaments useful for treating pain disorders, depressive disorders, affective disorders, attention deficit hyperactivity disorder, cognitive disorders, stress urinary incontinence, for inhibiting serotonin reuptake in a mammal, or for inhibiting norepinephrine reuptake in a mammal. Still another aspect of the invention relates to use of compounds of the invention as research tools. Other aspects and embodiments of the invention are disclosed herein.

### DETAILED DESCRIPTION OF THE INVENTION

### Definitions

When describing the compounds, compositions, methods and processes of the invention, the following terms have the following meanings unless otherwise indicated. Additionally, as used herein, the singular forms “a,” “an” and “the” include the corresponding plural forms unless the context of use clearly dictates otherwise. The terms “comprising”, “including,” and “having” are intended to be inclusive and mean that there may be additional elements other than the listed elements. All numbers expressing quantities of ingredients, properties such as molecular weight, reaction conditions, and so forth used herein are to be understood as being modified in all instances by the term “about,” unless otherwise indicated. Accordingly, the numbers set forth herein are approximations that may vary depending upon the desired properties sought to be obtained by the present invention. At least, and not as an attempt to limit the application of the doctrine of equivalents to the scope of the claims, each number should at least be construed in light of the reported significant digits and by applying ordinary rounding techniques.

The term “alkyl” means a monovalent saturated hydrocarbon group which may be linear or branched. Unless otherwise defined, such alkyl groups typically contain from 1 to 10 carbon atoms and include, for example, —C₁₋₂alkyl, —C₁₋₃alkyl, —C₁₋₄alkyl, —C₁₋₆alkyl, and —C₁₋₈alkyl. Representative alkyl groups include, by way of example, methyl, ethyl, n-propyl, isopropyl, n-butyl, sec-butyl, isobutyl, tert-butyl, n-pentyl, n-hexyl, n-heptyl, n-octyl, n-nonyl, n-decyl and the like.

When a specific number of carbon atoms is intended for a particular term used herein, the number of carbon atoms is shown preceding the term as subscript. For example, the term “—C₁₋₆alkyl” means an alkyl group having from 1 to 6 carbon atoms, and the term “—C₃₋₇cycloalkyl” means a cycloalkyl group having from 3 to 7 carbon atoms, respectively, where the carbon atoms are in any acceptable configuration.

The term “alkylene” means a divalent saturated hydrocarbon group that may be linear or branched. Unless otherwise defined, such alkylene groups typically contain from 0 to 10 carbon atoms and include, for example, —C₀₋₁alkylene, —C₀₋₂alkylene, —C₀₋₃alkylene, —C₀₋₆alkylene, —C₁₋₄alkylene, —C₂₋₄alkylene and —C₁₋₆alkylene. Representative alkylene groups include, by way of example, methylene, ethane-1,2-diyl (“ethylene”), propane-1,2-diyl, propane-1,3-diyl, butane-1,4-diyl, pentane-1,5-diyl and the like. It is understood that when the alkylene term includes zero carbons such as —C₀₋₁alkylene- or —C₀₋₃alkylene-, such terms are intended to include the absence of carbon atoms, that is, the alkylene group is not present except for a covalent bond attaching the groups separated by the alkylene term.

The term “alkynyl” means a monovalent unsaturated hydrocarbon group which may be linear or branched and which has at least one, and typically 1, 2 or 3, carbon-carbon triple bonds. Unless otherwise defined, such alkynyl groups typically contain from 2 to 10 carbon atoms and include, for example, —C₂₋₄alkynyl, —C₂₋₆alkynyl and —C₃₋₁₀alkynyl. Representative alkynyl groups include, by way of example, ethynyl, n-propynyl, n-but-2-ynyl, n-hex-3-ynyl and the like.

The term “cycloalkyl” means a monovalent saturated carbocyclic hydrocarbon group. Unless otherwise defined, such cycloalkyl groups typically contain from 3 to 10 carbon atoms and include, for example, —C₃₋₅cycloalkyl, —C₃₋₆cycloalkyl and —C₃₋₇cycloalkyl. Representative cycloalkyl groups include, by way of example, cyclopropyl, cyclobutyl, cyclopentyl, cyclohexyl and the like.

The term “halo” means fluoro, chloro, bromo and iodo.

The term “C₃₋₅heteroaryl” means a monovalent aromatic group having a single ring and containing in the ring one or two heteroatoms selected from nitrogen, oxygen, and sulfur. Unless otherwise defined, such heteroaryl groups typically contain from 5 to 7 total ring atoms. Typically, the point of attachment is at any available carbon atom. Representative C₃heteroaryl groups include, oxazole (e.g., 2-oxazole and 5-oxazole), isoxazole (e.g., 3-isoxazole), thiazole (e.g., 2-thiazole and 4-thiazole), isothiazole (e.g., 3-isothiazole), imidazole (e.g., 2-imidazole), and pyrazole (e.g., 1H-pyrazol-3-yl). Representative C₄heteroaryl groups include, thiophene (e.g., 2-thienyl), furan (e.g., 2-furyl and 3-furyl), pyrrole (e.g., 3-pyrrolyl and 2H-pyrrol-3-yl), pyrazine, pyrimidine (e.g., 2,6-pyrimidinyl and 3,5-pyrimidinyl), and pyridazine (e.g., 3-pyridazinyl). Representative C₅heteroaryl groups include pyridine (e.g., 2-pyridyl, 3-pyridyl, and 4-pyridyl) and pyran (e.g., 2H-pyran and 4H-pyran).

The term “pharmaceutically acceptable” refers to a material that is not biologically or otherwise unacceptable when used in the invention. For example, the term “pharmaceutically acceptable carrier” refers to a material that can be incorporated into a composition and administered to a patient without causing unacceptable biological effects or interacting in an unacceptable manner with other components of the composition. Such pharmaceutically acceptable materials typically have met the required standards of toxicological and manufacturing testing, and include those materials identified as suitable inactive ingredients by the U.S. Food and Drug Administration.

The term “pharmaceutically acceptable salt” means a salt prepared from a base or an acid which is acceptable for administration to a patient, such as a mammal (e.g., salts having acceptable mammalian safety for a given dosage regime). However, it is understood that the salts covered by the invention are not required to be pharmaceutically acceptable salts, such as salts of intermediate compounds that are not intended for administration to a patient. Pharmaceutically acceptable salts can be derived from pharmaceutically acceptable inorganic or organic bases and from pharmaceutically acceptable inorganic or organic acids. In addition, when a compound of formula I contains both a basic moiety, such as an amine, and an acidic moiety such as a carboxylic acid, zwitterions may be formed and are included within the term “salt” as used herein. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucoronic, mandelic, mucic, nicotinic, orotic, pamoic, pantothenic, sulfonic acids (e.g., benzenesulfonic, camphosulfonic, edisylic, ethanesulfonic, isethionic, methanesulfonic, naphthalenesulfonic, naphthalene-1,5-disulfonic, naphthalene-2,6-disulfonic and p-toluenesulfonic acids), xinafoic acid, and the like.

The term “solvate” means a complex or aggregate formed by one or more molecules of a solute, for example, a compound of formula I or a pharmaceutically acceptable salt thereof, and one or more molecules of a solvent. Such solvates are typically crystalline solids having a substantially fixed molar ratio of solute and solvent. Representative solvents include, by way of example, water, methanol, ethanol, isopropanol, acetic acid and the like. When the solvent is water, the solvate formed is a hydrate.

The term “therapeutically effective amount” means an amount sufficient to effect treatment when administered to a patient in need thereof, i.e., the amount of drug needed to obtain the desired therapeutic effect. For example, a therapeutically effective amount for treating neuropathic pain is an amount of compound needed to, for example, reduce, suppress, eliminate or prevent the symptoms of neuropathic pain or to treat the underlying cause of neuropathic pain. On the other hand, the term “effective amount” means an amount sufficient to obtain a desired result, which may not necessary be a therapeutic result. For example, when studying a system comprising a norepinephrine transporter, an “effective amount” may be the amount needed to inhibit norepinephrine reuptake.

The term “treating” or “treatment” as used herein means the treating or treatment of a disease or medical condition (such as neuropathic pain) in a patient, such as a mammal (particularly a human), that includes one or more of the following: (a) preventing the disease or medical condition from occurring, i.e., prophylactic treatment of a patient; (b) ameliorating the disease or medical condition, i.e., eliminating or causing regression of the disease or medical condition in a patient; (c) suppressing the disease or medical condition, i.e., slowing or arresting the development of the disease or medical condition in a patient; or (d) alleviating the symptoms of the disease or medical condition in a patient. For example, the term “treating neuropathic pain” would include preventing neuropathic pain from occurring, ameliorating neuropathic pain, suppressing neuropathic pain, and alleviating the symptoms of neuropathic pain. The term “patient” is intended to include those mammals, such as humans, that are in need of treatment or disease prevention, that are presently being treated for disease prevention or treatment of a specific disease or medical condition, as well as test subjects in which compounds of the invention are being evaluated or being used in a assay, for example an animal model.

All other terms used herein are intended to have their ordinary meaning as understood by those of ordinary skill in the art to which they pertain.

In one aspect, this invention relates to novel compounds of formula I:

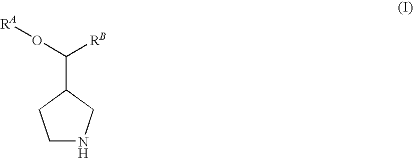

or a pharmaceutically acceptable salt thereof.

As used herein, the term “compound of the invention” or “compounds of the invention” include all compounds encompassed by formula I such as the species embodied in formula Ia-If, II-V, and all other subspecies of such formulas. In addition, when the compound of the invention contain a basic or acidic group (e.g., amino or carboxyl groups), the compound can exist as a free base, free acid, or in various salt forms. All such salt forms are included within the scope of the invention. Accordingly, those skilled in the art will recognize that reference to a compound herein, for example, reference to a “compound of the invention” or a “compound of formula I” includes a compound of formula I as well as pharmaceutically acceptable salts of that compound unless otherwise indicated. Furthermore, solvates of compounds of formula I are included within the scope of this invention.

The compounds of formula I contain at least two chiral centers and therefore, these compounds may be prepared and used in various stereoisomeric forms. Accordingly, the invention also relates to racemic mixtures, pure stereoisomers (e.g., enantiomers and diastereoisomers), stereoisomer-enriched mixtures, and the like unless otherwise indicated. When a chemical structure is depicted herein without any stereochemistry, it is understood that all possible stereoisomers are encompassed by such structure. Thus, for example, the terms “compound of formula I,” “compounds of formula II,” and so forth, are intended to include all possible stereoisomers of the compound. Similarly, when a particular stereoisomer is shown or named herein, it will be understood by those skilled in the art that minor amounts of other stereoisomers may be present in the compositions of the invention unless otherwise indicated, provided that the utility of the composition as a whole is not eliminated by the presence of such other isomers. Individual enantiomers may be obtained by numerous methods that are well known in the art, including chiral chromatography using a suitable chiral stationary phase or support, or by chemically converting them into diastereoisomers, separating the diastereoisomers by conventional means such as chromatography or recrystallization, then regenerating the original enantiomers. Additionally, where applicable, all cis-trans or E/Z isomers (geometric isomers), tautomeric forms and topoisomeric forms of the compounds of the invention are included within the scope of the invention unless otherwise specified.

More specifically, compounds of formula I contain at least two chiral centers indicated by the symbols * and ** in the following formula:

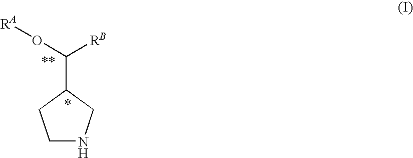

In one embodiment of the invention, the * chiral center has the (R) configuration, and the ** chiral center is undefined. This embodiment of the invention is shown in formula Ia:

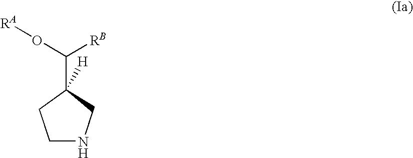

In another embodiment of the invention, the * chiral center has the (S) configuration, and the ** chiral center is undefined. This embodiment of the invention is shown in formula Ib:

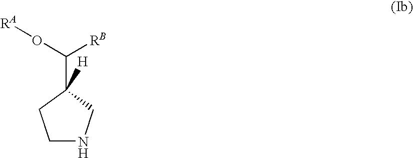

In one stereoisomer, both carbon atoms identified by the * and ** symbols have the (R) configuration. This embodiment of the invention is shown in formula Ic:

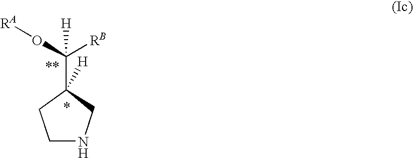

In this embodiment, compounds have the (R,R) configuration at the * and ** carbon atoms or are enriched in a stereoisomeric form having the (R,R) configuration at these carbon atoms.

In another stereoisomer, both carbon atoms identified by the * and ** symbols have the (S) configuration. This embodiment of the invention is shown in formula Id:

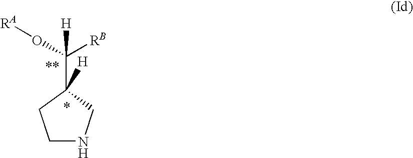

In this embodiment, compounds have the (S,S) configuration at the * and ** carbon atoms or are enriched in a stereoisomeric form having the (S,S) configuration at these carbon atoms.

In yet another stereoisomer, the carbon atom identified by the symbol * has the (S) configuration and the carbon atom identified by the symbol ** has the (R) configuration. This embodiment of the invention is shown in formula Ie:

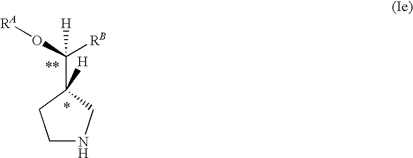

In this embodiment, compounds have the (S,R) configuration at the * and ** carbon atoms or are enriched in a stereoisomeric form having the (S,R) configuration at these carbon atoms.

In still another stereoisomer, the carbon atom identified by the symbol * has the (R) configuration and the carbon atom identified by the symbol ** has the (S) configuration. This embodiment of the invention is shown in formula If:

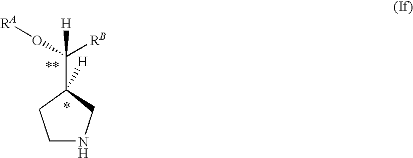

In this embodiment, compounds have the (R,S) configuration at the * and ** carbon atoms or are enriched in a stereoisomeric form having the (R,S) configuration at these carbon atoms.

Compounds of formula Ic and Id are enantiomers and therefore, in separate aspects, this invention relates to each individual enantiomer (i.e., Ic or Id), a racemic mixture of Ic and Id, or an enantiomer-enriched mixture of Ic and Id comprising predominately Ic or predominately Id. Similarly, compounds of formula Ie and If are enantiomers and therefore, in separate aspects, this invention relates to each individual enantiomer (i.e., Ie or If), a racemic mixture of Ie and If, or a enantiomer-enriched mixture of Ie and If comprising predominately Ie or predominately If.

In some embodiments, in order to optimize the therapeutic activity of the compounds of the invention, e.g., to treat neuropathic pain, it may be desirable that the carbon atoms identified by the * and ** symbols have a particular (R,R), (S,S), (S,R), or (R,S) configuration or are enriched in a stereoisomeric form having such configuration. For example, in one embodiment, the compounds of the invention have the (S,R) configuration of formula Ie or are enriched in a stereoisomeric form having the (S,R) configuration, and in another embodiment, the compounds of the invention have the (R,S) configuration of formula If, or are enriched in a stereoisomeric form having the (R,S) configuration. In other embodiments, the compounds of the invention are present as racemic mixtures, for example as a mixture of enantiomers of formula Ic and Id, or as a mixture of enantiomers of formula Ie and If.

This invention also includes isotopically-labeled compounds of formula I, i.e., compounds of formula I where one or more atoms have been replaced or enriched with atoms having the same atomic number but an atomic mass different from the atomic mass that predominates in nature. Examples of isotopes that may be incorporated into a compound of formula I include, but are not limited to ²H, ³H, ¹¹C, ¹³C, ¹⁴C, ¹³N, ¹⁵N, ¹⁵O, ¹⁷O, ¹⁸O, ³⁵S, ³⁶Cl, and ¹⁸F. Of particular interest are compounds of formula I enriched in tritium or carbon-14 which can be used, for example, in tissue distribution studies; compounds of formula I enriched in deuterium especially at a site of metabolism resulting, for example, in compounds having greater metabolic stability; and compounds of formula I enriched in a positron emitting isotope, such as ¹¹C, ¹⁸F, ¹⁵O and ¹³N, which can be used, for example, in Positron Emission Topography (PET) studies.

The compounds of the invention have been found to possess serotonin reuptake inhibitory activity and norepinephrine reuptake inhibitory activity. Among other properties, such compounds are expected to be useful as therapeutic agents for treating chronic pain, such as neuropathic pain. By combining dual activity into a single compound, double therapy can be achieved, i.e., serotonin reuptake inhibitory activity and norepinephrine reuptake inhibitory activity, using a single active component. Since pharmaceutical compositions containing one active component are typically easier to formulate than compositions containing two active components, such single-component compositions provide a significant advantage over compositions containing two active components.

Many combined serotonin and norepinephrine reuptake inhibitors (SNRIs) are more selective for SERT than for NET. For example, milnacipran, duloxetine, and venlafaxine and exhibit 2.5-fold, 10-fold, and 100-fold selectivity (measured as pKᵢ) for SERT over NET, respectively. Some, however, are less selective, such as bicifadine, which has a pKᵢ at SERT of 7.0 and a pKᵢ at NET of 6.7. Since it may be desirable to avoid selective compounds, in one embodiment of the invention the compounds have a more balanced SERT and NET activity, i.e., have the same pKᵢ value at both SERT and NET±0.5.

The nomenclature used herein to name the compounds of the invention is illustrated in the Examples herein. This nomenclature has been derived using the commercially-available AutoNom software (MDL, San Leandro, Calif.). Typically, compounds of formula I, have been named as 3-(phenoxypyrrolidin-3-yl-methyl)heteroaryls, 3-(phenylpyrrolidin-3-ylmethoxy)heteroaryls, and 3-(heteroarylpyrrolidin-3-ylmethoxy)heteroaryls.

### Representative Embodiments

The following substituents and values are intended to provide representative examples of various aspects and embodiments of the invention. These representative values are intended to further define and illustrate such aspects and embodiments and are not intended to exclude other embodiments or to limit the scope of the invention. In this regard, the representation that a particular value or substituent is preferred is not intended in any way to exclude other values or substituents from the invention unless specifically indicated.

As noted above, in one aspect, this invention relates to novel compounds of formula I:

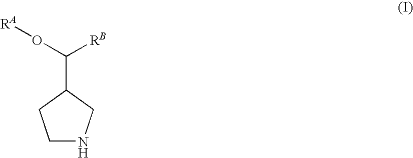

where R^{A} and R^{B} are independently C₃₋₅heteroaryl, naphthalene, or

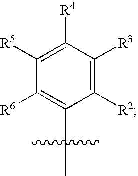

with the proviso that at least one of R^{A} and R^{B} is C₃₋₅heteroaryl or naphthalene; and further with the proviso that when R^{A} is:

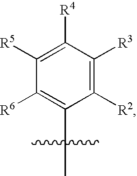

R^{B} is not unsubstituted 2-pyridine.

The C₃₋₅heteroaryl group and naphthalene groups are optionally substituted with one to four R¹ groups independently selected from halo, —C₁₋₆alkyl, —O—C₁₋₆alkyl, —O—C₃₋₇cycloalkyl, —CN, and —C₀₋₁alkylene-NR^{a}R^{b}. Further, the alkyl group may be substituted with 1 to 5 fluoro atoms. In one embodiment, the C₃₋₅heteroaryl group is selected from pyridine, thiazole, oxazole, furan, pyrazine, pyrimidine, and thiophene. In one embodiment, the C₃₋₅heteroaryl group is unsubstituted. In another embodiment, the C₃₋₅heteroaryl group is substituted with one to two R¹ groups independently selected from halo, —C₁₋₆alkyl, —O—C₁₋₆alkyl, —O—C₃₋₇cycloalkyl, —CN, and —C₀₋₁alkylene-NR^{a}R^{b}, where R^{a} and R^{b} are —C₁₋₄alkyl. The R^{A} and R^{B} C₃₋₅heteroaryl groups are attached to the core structure at a carbon atom. In one embodiment, the naphthalene group is unsubstituted. In another embodiment, the naphthalene group is substituted with one R¹ group selected from halo, —C₁₋₆alkyl, —O—C₁₋₆alkyl, where —C₁₋₆alkyl is optionally substituted with 1 to 5 fluoro atoms.

R² through R⁶ are independently selected from H, halo, —C₁₋₆alkyl, —C₂₋₆alkynyl, —O—C₁₋₆alkyl, —C₁₋₄alkylene-O—C₁₋₄alkyl, —C₃₋₇cycloalkyl, —C₀₋₁alkylene-phenyl, —O—C₀₋₃alkylene-phenyl, —C₀₋₆alkylene-OH, —CN, —C₀₋₂alkylene-COOH, —CHO, —C(O)—C₁₋₆alkyl, —C(O)O—C₁₋₄alkyl, —CH₂SH, —S—C₁₋₆alkyl, —C₁₋₄alkylene-S—C₁₋₄alkyl, —SO₂—C₁₋₆alkyl, —SO₂NR^{a}R^{b}, —NHSO₂R^{a}, —C₀₋₁alkylene-NR^{a}R^{b}, —NHC(O)—C₁₋₆alkyl, —C(O)NR^{a}R^{b}, and —NO₂. Further, each alkyl group in R² through R⁶ may be substituted with 1 to 5 fluoro atoms. In addition, each phenyl group in R² through R⁶ may be substituted with 1 or 2 groups independently selected from halo, —C₁₋₆alkyl, and —O—C₁₋₆alkyl.

The R^{a} and R^{b} groups are independently H or —C₁₋₄alkyl. It is understood that when referring to “each alkyl” group in R¹ or in R²⁻⁶, the term also includes any alkyl groups that might be present in the R^{a} and R^{b} moieties.

In some embodiments of the invention, one or more positions on the phenyl ring are substituted with a non-hydrogen moiety. For example, one such embodiment may be described by stating that that “R² is a non-hydrogen moiety”. It is understood that this means that R² can be any of the non-hydrogen moieties defined in formula I, i.e., halo, —C₁₋₆alkyl, —C₂₋₆alkynyl, —O—C₁₋₆alkyl, —C₁₋₄alkylene-O—C₁₋₄alkyl, —C₃₋₇cycloalkyl, —C₀₋₁alkylene-phenyl, —O—C₀₋₃alkylene-phenyl, —C₀₋₆alkylene-OH, —CN, —C₀₋₂alkylene-COOH, —CHO, —C(O)—C₁₋₆alkyl, —C(O)O—C₁₋₄alkyl, —CH₂SH, —S—C₁₋₆alkyl, —C₁₋₄alkylene-S—C₁₋₄alkyl, —SO₂—C₁₋₆alkyl, —SO₂NR^{a}R^{b}, —NHSO₂R^{a}, —C₀₋₁alkylene-NR^{a}R^{b}, —NHC(O)—C₁₋₆alkyl, —C(O)NR^{a}R^{b}, and —NO₂.

Exemplary halo groups include fluoro, chloro, bromo, and iodo. Exemplary —C₁₋₆alkyl groups include —CH₃, —CH₂CH₃, and —CH(CH₃)₂, as well as fluoro-substituted —C₁₋₆alkyl groups such as —CF₃. Exemplary —C₂₋₆alkynyl groups include —CH═CH₂. Exemplary —O—C₁₋₆alkyl groups include —OCH₃, —O—CH₂CH₃, and —OCH(CH₃)₂, as well as fluoro-substituted —O—C₁₋₆alkyl groups such as —OCF₃. Exemplary —C₁₋₄alkylene-O—C₁₋₄alkyl groups include —CH₂—OCH₃ and —CH₂—OCH₂CH₃. Exemplary —C₃₋₇cycloalkyl groups include cyclohexyl. Exemplary —C₀₋₁alkylene-phenyl groups include phenyl and benzyl. Exemplary —O—C₀₋₃alkylene-phenyl groups include —O-phenyl and —O-benzyl. As noted above, each phenyl group in R¹ and R² through R⁶ may be substituted with 1 or 2 groups independently selected from halo, —C₁₋₆alkyl, and —O—C₁₋₆alkyl. Examples of such substituted —O—C₀₋₃alkylene-phenyl groups include —O-2,4-dichlorophenyl, —O-3-chlorophenyl, —O-3-ethylphenyl, —O-4-ethylphenyl, —O-2-ethoxyphenyl, and —O-4-ethoxyphenyl. Exemplary —C₀₋₆alkylene-OH groups include —OH and —CH₂OH. Exemplary —C₀₋₂alkylene-COOH groups include —COOH. Exemplary —C(O)—C₁₋₆alkyl groups include —C(O)CH₃ and —C(O)CH₂CH₃. Exemplary —C(O)O—C₁₋₄alkyl groups include —C(O)OCH₃ and —C(O)OCH₂CH₃. Exemplary —S—C₁₋₆alkyl groups include —SCH₃. Exemplary —C₁₋₄alkylene-S—C₁₋₄alkyl groups include —CH₂—S—CH₃. Exemplary —SO₂—C₁₋₆alkyl groups include —SO₂CH₃. Exemplary —SO₂NR^{a}R^{b} groups include —SO₂NH₂ and —SO₂N(CH₃)₂. Exemplary —NHSO₂R^{a} groups include —NHSO₂H and —NHSO₂CH₃. Exemplary —C₀₋₁alkylene-NR^{a}R^{b} groups include —NH₂, —N(CH₃)₂, —CH₂NH(CH₂CH₃), and —CH₂N(CH₃)(CH₂CH₃). Exemplary —NHC(O)—C₁₋₆alkyl groups include —NHC(O)CH₃ and —NHC(O)CH₂CH₃. Exemplary —C(O)NR^{a}R^{b} groups include —CONH₂, —CONH(CH₂CH₃), and —C(O)N(CH₃)CH₂CH₃.

In one embodiment, R² is selected from H, halo, —C₁₋₆alkyl, —O—C₁₋₆alkyl, —C₃₋₇cycloalkyl, —C₀₋₁alkylene-phenyl, —O—C₀₋₃alkylene-phenyl, —C(O)—C₁₋₆alkyl, and —C(O)O—C₁₋₄alkyl, where each alkyl is optionally substituted with 1 to 5 fluoro atoms. In another embodiment, R³ is selected from H, halo, and —C₁₋₆alkyl. In one embodiment, R⁴ is selected from H, halo, —C₁₋₆alkyl, —O—C₁₋₆alkyl, and —C₀₋₁alkylene-phenyl. In yet another embodiment, R⁵ is selected from H, halo, and —C₁₋₆alkyl. In still another embodiment, R⁶ is selected from H, halo, and —C₁₋₆alkyl.

In another embodiment of the compound of formula I, R^{A} and R^{B} are independently selected from 2-pyridine, 3-pyridine, 4-pyridine, thiazole, oxazole, furan, pyrazine, pyrimidine, naphthalene, and

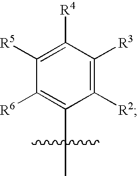

where each pyridine and naphthalene group is optionally substituted with one to four R¹ groups independently selected from halo, —C₁₋₆alkyl, —O—C₁₋₆alkyl, —O—C₃₋₇cycloalkyl, —CN, and —C₀₋₁alkylene-NR^{a}R^{b}, where R^{a} and R^{b} are —C₁₋₄alkyl; and R² through R⁶ are independently selected from H, halo, —C₁₋₆alkyl, —O—C₁₋₆alkyl, —C₃₋₇cycloalkyl, —C₀₋₁alkylene-phenyl, —O—C₀₋₃alkylene-phenyl, —C(O)—C₁₋₆alkyl, and —C(O)O—C₁₋₄alkyl; and each alkyl is optionally substituted with 1 to 5 fluoro atoms. In one particular embodiment, each pyridine and naphthalene group is optionally substituted with one to four R¹ groups independently selected from halo, —C₁₋₆alkyl, —O—C₁₋₆alkyl, and —O—C₃₋₇cycloalkyl; R² is H, halo, —C₁₋₆alkyl, —O—C₁₋₆alkyl, —C₃₋₇cycloalkyl, —C₀₋₁alkylene-phenyl, —C(O)—C₁₋₆alkyl, or —C(O)O—C₁₋₄alkyl; R³ is H, halo, or —C₁₋₆alkyl; R⁴ is H, halo, —C₁₋₆alkyl, or —C₀₋₁alkylene-phenyl; R⁵ is H or halo; R⁶ is H, halo, or —C₁₋₆alkyl; and each alkyl is optionally substituted with 1 to 5 fluoro atoms. In yet another particular embodiment, each pyridine group is optionally substituted with one to four R¹ groups independently selected from halo, —C₁₋₆alkyl, —O—C₁₋₆alkyl, and —O—C₃₋₇cycloalkyl; R² is H, halo, —C₁₋₆alkyl, —O—C₁₋₆alkyl, —C₀₋₁alkylene-phenyl, —C(O)—C₁₋₆alkyl, or —C(O)O—C₁₋₄alkyl; R³ and R⁴ are independently H, halo, or —C₁₋₆alkyl; and R⁵ and R⁶ are independently H or halo; and each alkyl is optionally substituted with 1 to 5 fluoro atoms.

In one embodiment of the invention, R^{A} is:

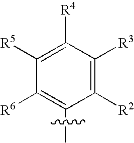

and R^{B} is a C₃₋₅heteroaryl, other than unsubstituted 2-pyridine. This embodiment of the invention is shown in formula II:

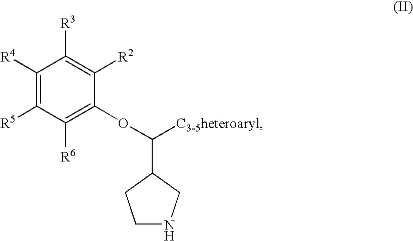

where C₃₋₅heteroaryl is selected from 2-pyridine substituted with one to two R¹ groups, 3-pyridine optionally substituted with one to two R¹ groups, 4-pyridine optionally substituted with one to two R¹ groups, thiazole, oxazole, furan, pyrazine, and pyrimidine; and R¹-R⁶ are as defined for formula I. In one embodiment of the compound of formula II, R^{B} is selected from 3-pyridine substituted with one halo group, 4-pyridine substituted with one halo group, thiazole, oxazole, furan, pyrazine, and pyrimidine; and R²-R⁶ are as defined for formula I. In yet another embodiment of the compound of formula II, R^{B} is thiazole or oxazole; and R²-R⁶ are as defined for formula I. In one particular embodiment of the compound of formula II, R² is H, halo, —C₁₋₆alkyl, —O—C₁₋₆alkyl, —C₃₋₇cycloalkyl, —C₀₋₁alkylene-phenyl, —O—C₀₋₃alkylene-phenyl, —C(O)—C₁₋₆alkyl, or —C(O)O—C₁₋₄alkyl, where the alkyl in —C₁₋₆alkyl is optionally substituted with 1 to 5 fluoro atoms; R³ is H, halo, or —C₁₋₆alkyl; R⁴ is H, halo, —C₁₋₆alkyl, —O—C₁₋₆alkyl, or —C₀₋₁alkylene-phenyl; R⁵ is H or halo; and R⁶ is H, halo, or —C₁₋₆alkyl.

In embodiment of the compound of formula II, R^{B} is 2-pyridine substituted with one to four R¹ groups, 3-pyridine optionally substituted with one to four R¹ groups, or 4-pyridine optionally substituted with one to four R¹ groups:

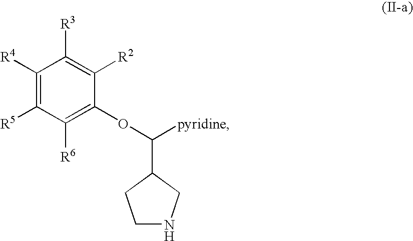

where R¹-R⁶ are as defined for formula I. In one particular embodiment of the compound of formula II-a, R^{B} is 3-pyridine optionally substituted with one halo group, or 4-pyridine; R² is H, halo, —C₁₋₆alkyl, —O—C₁₋₆alkyl, —C₀₋₁alkylene-phenyl, —C(O)—C₁₋₆alkyl, or —C(O)O—C₁₋₄alkyl, where the alkyl in —C₁₋₆alkyl is optionally substituted with 1 to 5 fluoro atoms; R³ is H, halo, or —C₁₋₆alkyl; R⁴ is H, halo, —C₁₋₆alkyl, or —C₀₋₁alkylene-phenyl; R⁵ is H or halo; and R⁶ is H, halo, or —C₁₋₆alkyl. In another embodiment of the compound of formula II-a, the pyridine is 3-pyridine, as depicted in formula II-a1:

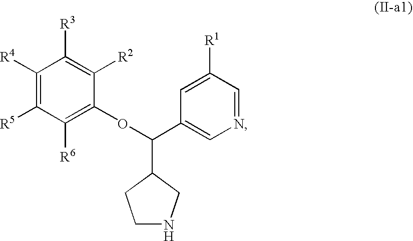

where R¹-R⁶ are as defined for formula I. In one embodiment of the compound of formula II-a1, R¹ is Cl; R² is H, halo, —C₁₋₆alkyl, —O—C₁₋₆alkyl, —C₀₋₁alkylene-phenyl, —C(O)—C₁₋₆alkyl, or —C(O)O—C₁₋₄alkyl, where the alkyl in —C₁₋₆alkyl is optionally substituted with 1 to 5 fluoro atoms; R³ is H or halo; R⁴ is H or halo; R⁵ is H or halo; and R⁶ is H or halo. In another embodiment of the compound of formula II-a1, R¹ is F; R² is H, halo, —C₁₋₆alkyl, —O—C₁₋₆alkyl, —C₀₋₁alkylene-phenyl, —C(O)—C₁₋₆alkyl, or —C(O)O—C₁₋₄alkyl, where the alkyl in —C₁₋₆alkyl is optionally substituted with 1 to 5 fluoro atoms; R³ is H or halo; R⁴ is H, halo, —C₁₋₆alkyl, or —C₀₋₁alkylene-phenyl; R⁵ is H or halo; and R⁶ is H, halo, or —C₁₋₆alkyl. In one embodiment of the compound of formula II-a1, R¹ is hydrogen; R² is halo, —C₁₋₆alkyl, or —O—C₁₋₆alkyl; R³ is H, halo, or —C₁₋₆alkyl; R⁴ is H or halo; R⁵ is H; and R⁶ is H or halo.

In another embodiment of the compound of formula IIa, the pyridine is 4-pyridine, as depicted in formula II-a3:

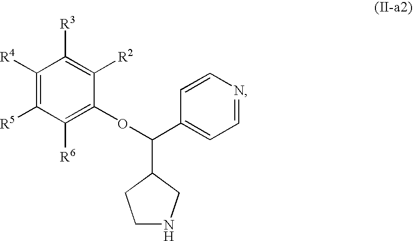

where R²-R⁶ are as defined for formula I. In one embodiment of the compound of formula II-a3, R² is halo; R³ is H or —C₁₋₆alkyl; R⁴ is H or halo; R⁵ is H; and R⁶ is halo.

In another embodiment of the compound of formula II, R^{B} is thiazole:

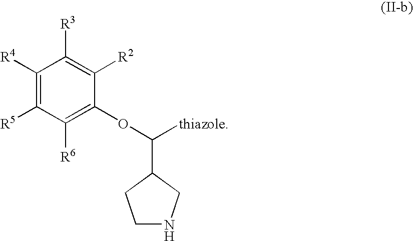

where R²-R⁶ are as defined for formula I. In one embodiment of the compound of formula II-b, R² is H, halo, or —C₁₋₆alkyl; R³ is H, halo, or —C₁₋₆alkyl; R⁴ is H, halo, —C₁₋₆alkyl, or —O—C₁₋₆alkyl; R⁵ is H or halo; and R⁶ is H or halo. The thiazole can be:

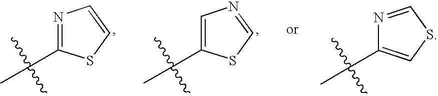

In one particular embodiment of the compound of formula II-b, the thiazole is 2-thiazole, as depicted in formula II-b1:

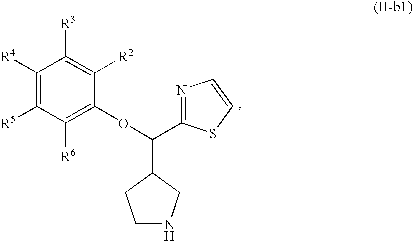

where R²-R⁶ are as defined for formula I. In one embodiment of the compound of formula II-b1, R² is H, halo, or —C₁₋₆alkyl; R³ is H, halo, or —C₁₋₆alkyl; R⁴ is H or halo; R⁵ is H or halo; and R⁶ is H or halo.

In another embodiment of the compound of formula II, R^{B} is oxazole:

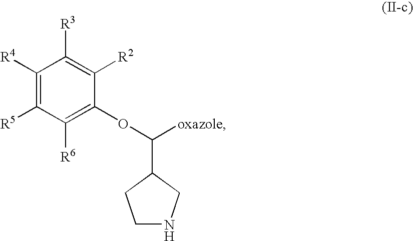

where R²-R⁶ are as defined for formula I. In one embodiment of the compound of formula II-c, R² is H, halo, —C₁₋₆alkyl, —O—C₁₋₆alkyl, —C₃₋₇cycloalkyl, —C₀₋₁alkylene-phenyl, —O—C₀₋₃alkylene-phenyl, —C(O)—C₁₋₆alkyl, or —C(O)O—C₁₋₄alkyl, where each alkyl is optionally substituted with 1 to 5 fluoro atoms; R³ is H, halo, or —C₁₋₆alkyl; R⁴ is H, halo, —C₁₋₆alkyl, or —C₀₋₁alkylene-phenyl; R⁵ is H or halo; and R⁶ is H, halo, or —C₁₋₆alkyl. The oxazole can be:

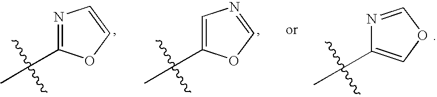

In one particular embodiment of the compound of formula II-c, the oxazole is 2-oxazole, as depicted in formula II-c1:

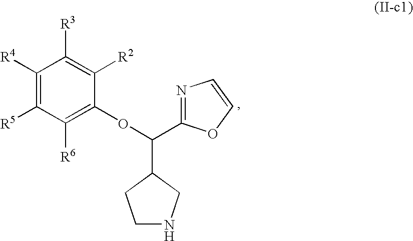

where R²-R⁶ are as defined for formula I. In one particular embodiment of the compound of formula II-c1, R² is H, halo, —C₁₋₆alkyl, —O—C₁₋₆alkyl, —C₃₋₇cycloalkyl, —C₀₋₁alkylene-phenyl, —C(O)—C₁₋₆alkyl, or —C(O)O—C₁₋₄alkyl, where the alkyl in —C₁₋₆alkyl is optionally substituted with 1 to 5 fluoro atoms; R³ is H or halo; R⁴ is H, halo, or —C₁₋₆alkyl; R⁵ is H or halo; and R⁶ is H, halo, or —C₁₋₆alkyl.

In another embodiment of the compound of formula II-c, the oxazole is 5-oxazole, as depicted in formula II-c2:

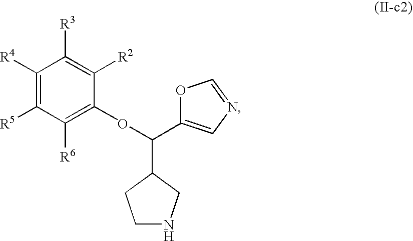

where R²-R⁶ are as defined for formula I. In one particular embodiment of the compound of formula II-c2, R² is halo or —C₁₋₆alkyl; R³ is H or halo; R⁴ is H or halo; R⁵ is H; and R⁶ is H or halo.

In another embodiment of the compound of formula II, R^{B} is furan:

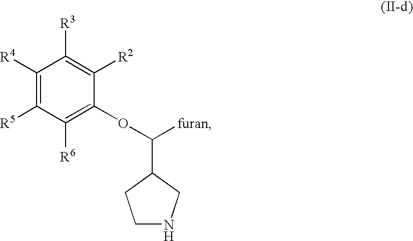

where R²-R⁶ are as defined for formula I. In one embodiment of the compound of formula II-d, R² is H, halo, or —C₁₋₆alkyl; R³ is H or halo; R⁴ is H or halo; R⁵ is H or halo; and R⁶ is H or halo. In one embodiment of the compound of formula II-d, the furan is 2-furyl, as depicted in formula II-d1:

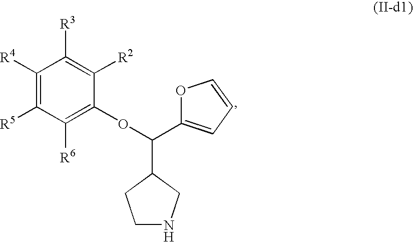

where R²-R⁶ are as defined for formula I. In another embodiment of the compound of formula IId, the furan is 3-furyl, as depicted in formula II-d2:

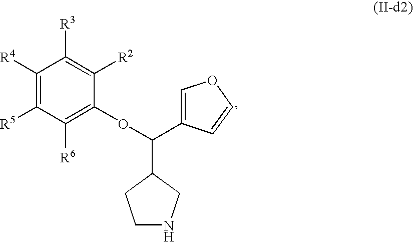

where R²-R⁶ are as defined for formula I. In one embodiment of the compound of formula II-d1 or II-d2, R² is H; R³ is halo; R⁴ is H; R⁵ is halo; and R⁶ is H.

In another embodiment of the compound of formula II, R^{B} is pyrazine, as depicted in formula II-e:

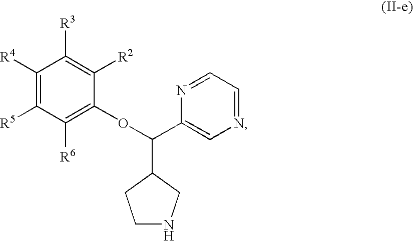

where R²-R⁶ are as defined for formula I. In one embodiment of the compound of formula II-e, R² is H, halo, —C₁₋₆alkyl, or —O—C₁₋₆alkyl; R³ is H, halo, or —C₁₋₆alkyl; R⁴ is H or halo; R⁵ is H or halo; and R⁶ is H or halo. In one particular embodiment of the compound of formula II-e, R² is H, halo, or —C₁₋₆alkyl; R³ is H, halo, or —C₁₋₆alkyl; R⁴ is H or halo; R⁵ is H or halo; and R⁶ is H or halo.

In another embodiment of the compound of formula II, R^{B} is pyrimidine.

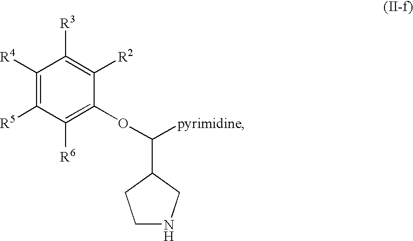

where R²-R⁶ are as defined for formula I. In one embodiment of the compound of formula II-f, R² is H, halo, —C₁₋₆alkyl, or —O—C₁₋₆alkyl; R³ is H or halo; R⁴ is H or halo; R⁵ is H or halo; and R⁶ is H or halo. In one embodiment of the compound of formula II-f, the pyrimidine is 2,6-pyrimidinyl, as depicted in formula II-f1:

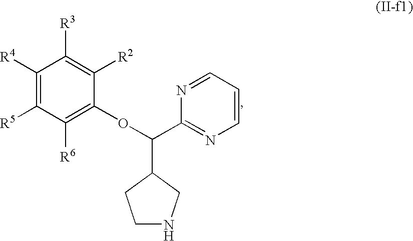

where R²-R⁶ are as defined for formula I. In another embodiment of the compound of formula IIf, the pyrimidine is 3,5-pyrimidinyl, as depicted in formula II-f2:

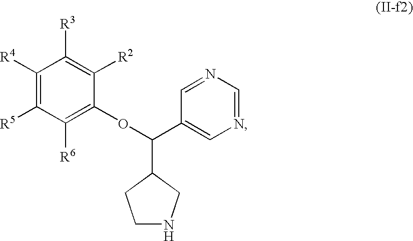

where R²-R⁶ are as defined for formula I. In one embodiment of the compound of formula II-II-f1 or II-f2, R² is —C₁₋₆alkyl; R³ is H; R⁴ is halo; R⁵ is H; and R⁶ is H.

In another embodiment of the invention, R^{A} is C₃₋₅heteroaryl and R^{B} is:

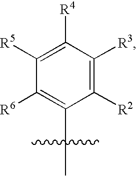

where R²-R⁶ are as defined for formula I. This embodiment of the invention is shown in formula III:

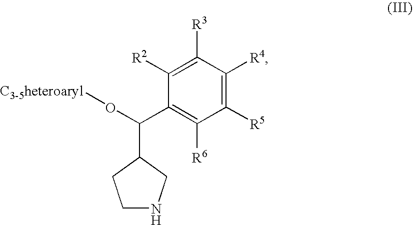

where C₃₋₅heteroaryl, and R²-R⁶ are as defined for formula I. In one embodiment of the compound of formula III, R^{A} is pyridine:

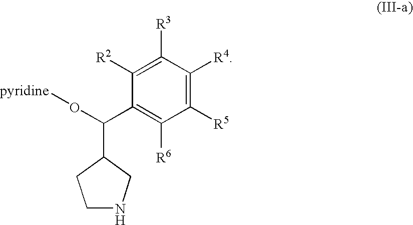

where R²-R⁶ are as defined for formula I. In one embodiment of the compound of formula III-a, the pyridine is optionally substituted with one to four R¹ groups independently selected from halo, —C₁₋₆alkyl, —O—C₁₋₆alkyl, —O—C₃₋₇cycloalkyl, —CN, and —C₀₋₁alkylene-NR^{a}R^{b}, where R^{a} and R^{b} are —C₁₋₄alkyl; where each alkyl is optionally substituted with 1 to 5 fluoro atoms; and R²-R⁶ are H. In another embodiment of the compound of formula III-a, the pyridine is 2-pyridine, as depicted in formula III-a1:

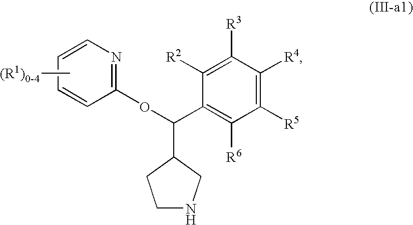

where R¹-R⁶ are as defined for formula I. In one embodiment of the compound of formula III-a1, the pyridine is optionally substituted with one to four R¹ groups independently selected from halo, —C₁₋₆alkyl, —O—C₁₋₆alkyl, —CN, and —C₀₋₁alkylene-NR^{a}R^{b}, where R^{a} and R^{b} are —C₁₋₄alky, where the alkyl in —C₁₋₆alkyl is optionally substituted with 1 to 5 fluoro atoms. In one particular embodiment of the compound of formula III-a1, the pyridine is optionally substituted with one to four R¹ groups independently selected from halo, —C₁₋₆alkyl, and —O—C₁₋₆alkyl, where the alkyl in —C₁₋₆alkyl is optionally substituted with 1 to 5 fluoro atoms.

In one embodiment of the compound of formula III-a, the pyridine is 3-pyridine, as depicted in formula III-a2:

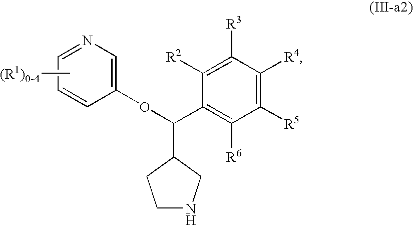

where R¹-R⁶ are as defined for formula I. In one embodiment of the compound of formula III-a2, the pyridine is optionally substituted with one or two R¹ groups independently selected from halo, —C₁₋₆alkyl, and —O—C₁₋₆alkyl, —O—C₃₋₇cycloalkyl, where the alkyl in —O—C₁₋₆alkyl is optionally substituted with 1 to 5 fluoro atoms. In one particular embodiment of the compound of formula III-a2, the pyridine is substituted with one or two R¹ groups independently selected from halo, —O—C₁₋₆alkyl, and —O—C₃₋₇cycloalkyl, where the alkyl in —O—C₁₋₆alkyl is optionally substituted with 1 to 5 fluoro atoms.

In one embodiment of the compound of formula III-a, the pyridine is 4-pyridine, as depicted in formula III-a3:

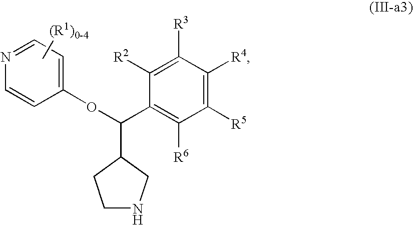

where R¹-R⁶ are as defined for formula I. In one embodiment of the compound of formula III-a3, the pyridine is substituted with one or two R¹ groups independently selected from halo, —C₁₋₆alkyl, and —CN.

In another embodiment of the invention, R^{A} and R^{B} are independently C₃₋₅heteroaryl. This embodiment of the invention is shown in formula IV:

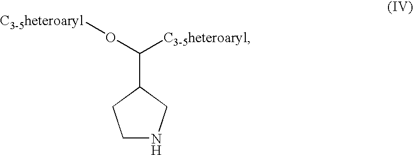

where the C₃₋₅heteroaryl groups are as defined for formula I. In one embodiment of the compound of formula IV, R^{A} is 5-chloro-3-methyl-2-pyridine, as depicted in formula IV-a:

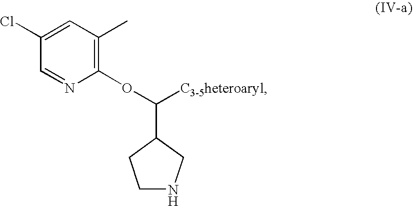

where the C₃₋₅heteroaryl group is as defined for formula I. In one particular embodiment of the compound of formula IV-a, R^{B} is 2-oxazole, 5-fluoro-3-pyridine, or 5-clororo-3-pyridine.

In another embodiment of the invention, R^{A} is naphthalene and R^{B} is C₃₋₅heteroaryl. This embodiment of the invention is shown in formula V:

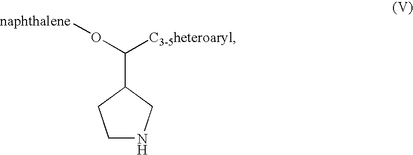

where the C₃₋₅heteroaryl group is as defined for formula I. In one embodiment of the compound of formula V, R^{A} is naphthalen-1-yl or naphthalen-2-yl, and R^{B} is 2-oxazole, 3-chloro-5-pyridine, or 3-fluoro-5-pyridine. In one embodiment of the compound of formula V, the naphthalene is 1-naphthalene, as depicted in formula V-a:

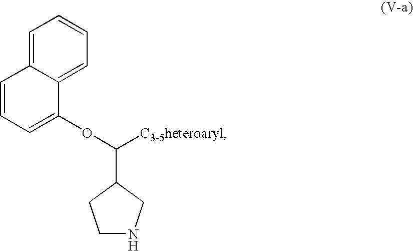

where the C₃₋₅heteroaryl group is defined for formula I. In another embodiment of the compound of formula V, the naphthalene is 2-naphthalene, as depicted in formula V-b:

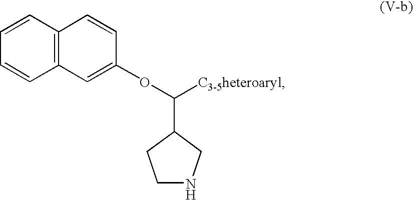

where the C₃₋₅heteroaryl group is defined for formula I.

In another embodiment of the invention, R^{A} is naphthalene and R^{B} is

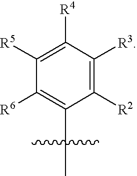

This embodiment of the invention is shown in formula VI:

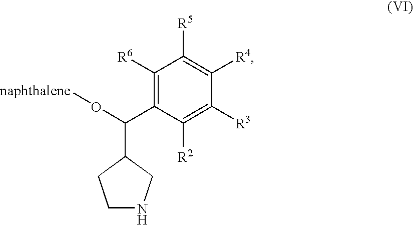

where R²-R⁶ are as defined for formula I. In one embodiment of the compound of formula VI, R^{A} is naphthalen-1-yl, optionally substituted with one R¹ group, as depicted in formula VI-a:

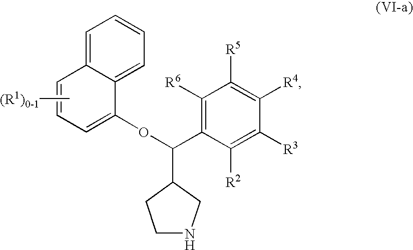

where R¹⁻⁶ are as defined for formula I. In another embodiment, R¹ is halo or —O—C₁₋₆alkyl; R² is H or —C₁₋₆alkyl; R³ is H, halo, —C₁₋₆alkyl, or —O—C₁₋₆alkyl; R⁴, R⁵ and R⁶ are H; and each alkyl is optionally substituted with 1 to 5 fluoro atoms. In another embodiment, R¹ is halo or —O—C₁₋₆alkyl; R² is H or —C₁₋₆alkyl; R³ is H, halo, —C₁₋₆alkyl, or —O—C₁₋₆alkyl; and R⁴, R⁵ and R⁶ are H.

In another embodiment of the invention, R^{A} is C₃₋₅heteroaryl and R^{B} is naphthalene. In yet another embodiment of the invention, R^{A} is:

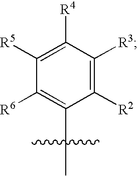

and R^{B} is naphthalene. In still another embodiment of the invention, R^{A} and R^{B} are naphthalene.

In one embodiment, the compounds of the invention exhibit a SERT and NET pKᵢ≧7.0, and in some embodiments have a SERT and NET pKᵢ≧8.0.

In addition, particular compounds of formula I that are of interest include those set forth in the Examples below, as well pharmaceutically acceptable salts thereof.

### General Synthetic Procedures

Compounds of the invention can be prepared from readily available starting materials using the following general methods, the procedures set forth in the Examples, or by using other methods, reagents, and starting materials that are known to those skilled in the art. Although the following procedures may illustrate a particular embodiment of the invention, it is understood that other embodiments of the invention can be similarly prepared using the same or similar methods or by using other methods, reagents and starting materials known to those of ordinary skill in the art. It will also be appreciated that where typical or preferred process conditions (i.e., reaction temperatures, times, mole ratios of reactants, solvents, pressures, etc.) are given, other process conditions can also be used unless otherwise stated. While optimum reaction conditions will typically vary depending on various reaction parameters such as the particular reactants, solvents and quantities used, those of ordinary skill in the art can readily determine suitable reaction conditions using routine optimization procedures.

Additionally, as will be apparent to those skilled in the art, conventional protecting groups may be necessary or desired to prevent certain functional groups from undergoing undesired reactions. The choice of a suitable protecting group for a particular functional group as well as suitable conditions and reagents for protection and deprotection of such functional groups are well-known in the art. Protecting groups other than those illustrated in the procedures described herein may be used, if desired. For example, numerous protecting groups, and their introduction and removal, are described in Greene and Wuts, Protecting Groups in Organic Synthesis, Third Edition, Wiley, New York, 1999, and references cited therein.

More particularly, in the schemes below, P represents an “amino-protecting group,” a term used herein to mean a protecting group suitable for preventing undesired reactions at an amino group. Representative amino-protecting groups include, but are not limited to, t-butoxycarbonyl (BOC), trityl (Tr), benzyloxycarbonyl (Cbz), 9-fluorenylmethoxycarbonyl (Fmoc), formyl, and the like. Standard deprotection techniques and reagents such as TFA in DCM or HCl in 1,4-dioxane, methanol, or ethanol, are used to remove protecting groups, when present. For example, a BOC group can be removed using an acidic reagent such as hydrochloric acid, trifluoroacetic acid and the like; while a Cbz group can be removed by employing catalytic hydrogenation conditions such as H₂ (1 atm), 10% Pd/C in an alcoholic solvent.

Suitable inert diluents or solvents for use in these schemes include, by way of illustration and not limitation, tetrahydrofuran (THF), acetonitrile, N,N-dimethylformamide (DMF), dimethylsulfoxide (DMSO), toluene, dichloromethane (DCM), chloroform (CHCl₃), and the like.

All reactions are typically conducted at a temperature within the range of about −78° C. to 110° C., for example at room temperature. Reactions may be monitored by use of thin layer chromatography (TLC), high performance liquid chromatography (HPLC), and/or LCMS until completion. Reactions may be complete in minutes, may take hours, typically from 1-2 hours and up to 48 hours, or days, such as up to 3-4 days. Upon completion, the resulting mixture or reaction product may be further treated in order to obtain the desired product. For example, the resulting mixture or reaction product may be subjected to one or more of the following procedures: dilution (for example with saturated NaHCO₃); extraction (for example, with ethyl acetate, CHCl₃, DCM, aqueous HCl); washing (for example, with DCM, saturated aqueous NaCl, or saturated aqueous NaHCO₃); drying (for example, over MgSO₄ or Na₂SO₄, or in vacuo); filtration; being concentrated (for example, in vacuo); being redissolved (for example in a 1:1 acetic acid:H₂O solution); and/or purification (for example by preparative HPLC, reverse phase preparative HPLC, or crystallization).

By way of illustration, compounds of formula I, as well as their salts, can be prepared by one or more of the following schemes. Scheme A exemplifies the preparation of the alcohol intermediate for those compounds where R^{B} is C₃₋₅heteroaryl, e.g., compounds of formula II, IV, and V.

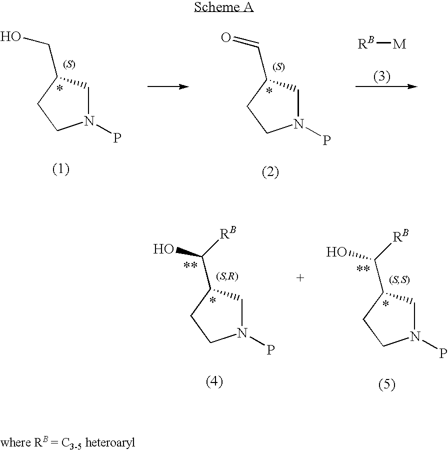

Note that the * chiral center shown in Scheme A is known to be S. However, the ** chiral center shown in Scheme A is not known unambiguously and was designated R or S based upon the first elution peak by reverse phase HPLC from the mixture of diastereomeric protected alcohols. The first eluting peak was designated R at the ** chiral center and the second eluting peak was designated S at the ** chiral center. Furthermore, while Scheme A illustrates formation of the (S,R) and (S,S) alcohols 4 and 5, respectively, the (R,S) and (R,R) alcohols can be made in a similar manner, using the (R) stereoisomer compound 1′ as the starting material:

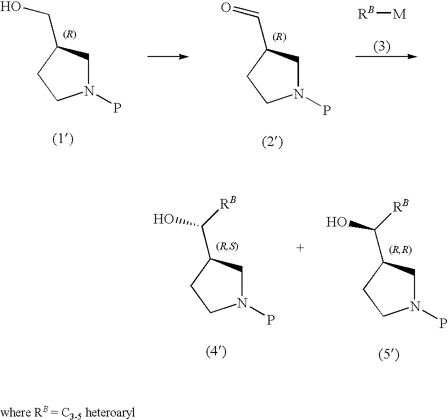

Compound 2 can be prepared by oxidizing compound 1 using any oxidizing agent suitable for converting a primary alcohol into an aldehyde. Representative oxidizing agents include, for example, dimethyl sulfoxide, Collin's reagent, Corey's reagent, pyridinium dichromate and the like. In one embodiment, compound 2 is prepared by the 2,2,6,6-tetramethyl-1-piperidinyloxy, free radical (TEMPO) mediated oxidation of compound 1. This method is particularly useful by minimizing the amount of racemization that can occur when the alcohol 1 or 1′ is oxidized. Compound 1, where P is Boc or benzyl, is commercially available. Compound 2′ can be prepared in a similar manner, using R-Boc-3-pyrrolidinemethanol, also known as (R)-3-hydroxymethylpyrrolidine-1-carboxylic acid t-butyl ester, as starting material compound V.

Compound (3) is a C₃₋₅heteroaryl-containing organometallic reagent, where M can represent magnesium, lithium, zinc, trimethylsilyl, and so forth. Compound (3) is typically commercially available or can be prepared by techniques that are known in the art.

In Scheme A, the Grignard reaction between compound 2 and compound 3 is typically conducted using standard Grignard reaction conditions. For example, compound 3, in a suitable solvent such as THF, is combined with an alkyl Grignard reagent such as isopropyl magnesium chloride or butyl magnesium chloride in a suitable solvent such as THF. The reaction is allowed to progress, while the mixture is stirred. Compound 2 is added, the mixture is stirred to allow the reaction to proceed. typically for several hours. The reaction is then quenched, for example using saturated ammonium chloride. Purification and separation by preparative HPLC or crystallization will then yield compounds 4 and 5. Similarly, compound 2′ as the starting material will yield compounds 4′ and 5′. Compounds 4 and/or 5 can also be prepared by reduction of the corresponding ketone.

Scheme B exemplifies the preparation of the alcohol intermediate for those compounds where R^{B} is naphthalene, or:

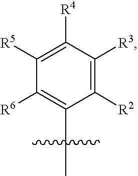

e.g., compounds of formula III and VI.

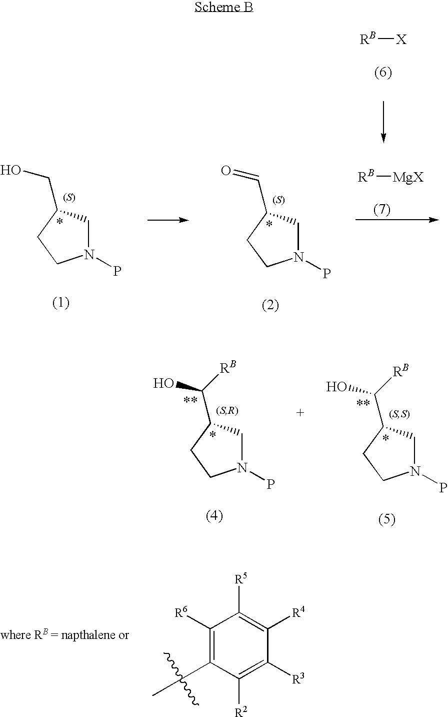

Note that the * chiral center shown in Scheme B is known to be S. The ** chiral center shown in Scheme B is also known, for compounds where R^{B} is the optionally substituted phenyl group. However, for compounds where R^{B} is naphthalene, the ** chiral center shown in Scheme B is not known unambiguously and was designated R or S based upon the first elution peak by reverse phase HPLC from the mixture of diastereomeric protected alcohols. The first eluting peak was designated R at the ** chiral center and the second eluting peak was designated S at the ** chiral center. Furthermore, as with Scheme A, while Scheme B illustrates formation of the (S,R) enantiomer of the compound of formula III, the (R,S) enantiomer can be made in a similar manner.

### Preparation of Compound 7

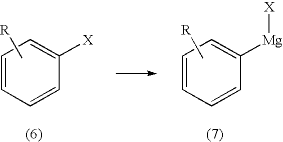

Compound 7 is a Grignard reagent, and serves to introduce an unsubstituted (a=0) or substituted phenyl group into compound 2. Compound 7, where R^{B} is an optionally substituted phenyl, can be readily prepared by treating a compound 6 (for example, where X is bromo or iodo) with magnesium metal. See for example, Knochel et al. (2003) Angew. Chem. Int. Ed. 42(36):4302-4320. Compound 7 may also be commercially available, examples of which include phenyl magnesium bromide. Compound 7, where R^{B} is naphthalene, can be prepared in a similar manner.

Alternately, other reagents can be used to introduce the phenyl group into compound 2 or 2′. For example, compound 7 can be replaced with:

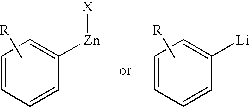

in Scheme II. Both compounds are commercially available or can be prepared by techniques that are known in the art.

In Scheme B, the Grignard reaction between compound 2 and compound 7 is typically conducted using standard Grignard reaction conditions. For example, compound 2, in a suitable solvent such as THF, is cooled under nitrogen to about −78° C. Compound 7 in a suitable solvent such as THF is added dropwise and the solution is allowed to warm to room temperature, typically overnight. The reaction is then quenched, for example using saturated NH₄Cl. Purification and separation by preparative HPLC or crystallization will then yield compounds 4 and 5.

Scheme C exemplifies the preparation of compounds where R^{A} is C₃₋₅heteroaryl, e.g., compounds of formula III and IV.

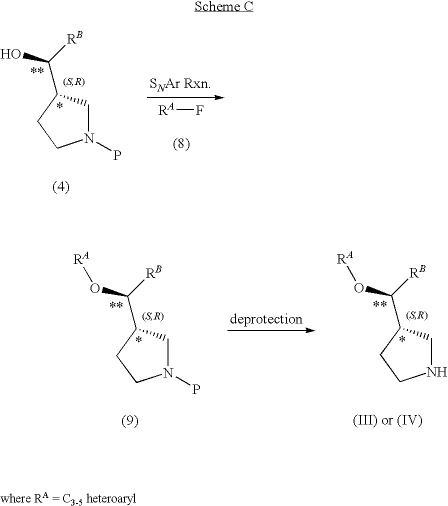

In Scheme C, compound 9 is prepared by reacting compound 4 with an appropriate heteroaryl fluoride (compound 8) using a nucleophilic aromatic substitution reaction (SNAr). For example, sodium hydride is slowly added to compound 4 that has been dissolved in an appropriate solvent such as DMF. An appropriate heteroaryl fluoride (compound 8) is then added and the mixture is stirred at about 70° C. until the reaction is complete, yielding compound 9, which is then deprotected to yield the compound of formula III of IV. In a similar manner, the (R,S) form of compound III or IV can be prepared by starting with the (R,S) alcohol intermediate.

Scheme D exemplifies one method of preparing compounds where R^{A} is naphthalene or:

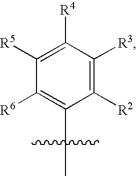

e.g., compounds formula II, V, and VI.

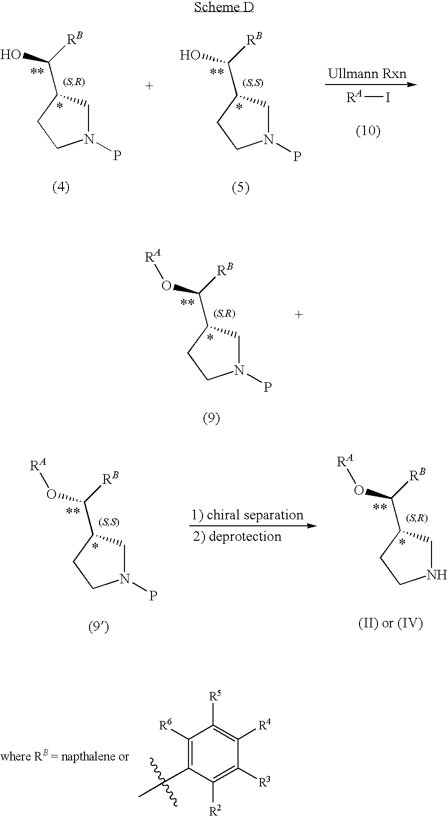

In Scheme D, compounds 4 and 5 are coupled to an appropriate aryl iodide (compound 10) under Ullmann reaction conditions to provide a mixture of compounds 9 and 9′. The Ullmann reaction is typically conducted in the presence of a copper(I) iodide/1,10-phenanthroline catalyst and a base such as cesium carbonate, in an appropriate solvent such as toluene or DMF. The reaction vessel is sealed and the mixture is heated at about 100-110° C. until the reaction is complete, typically about 2-3 days, yielding compounds 9 and 9′. This mixture is then purified and separated by normal phase chiral HPLC. The desired separated compound is then deprotected to yield the compound of formula II or IV. This final step is conducted under standard deprotection conditions, which will vary depending upon the protecting group used. For example, removal of the BOC group can be done using HCl and ethanol. Alternately, the mixture of compounds 9 and 9′ can first be deprotected, then separated by normal phase chiral HPLC to yield the compound of formula II or IV.

Compounds of formula II or IV can also be prepared using the Mitsunobu coupling reaction (Mitsunobu and Yamada (1967) M. Bull. Chem. Soc. JPN. 40:2380-2382). This reaction is typically conducted using standard Mitsunobu coupling conditions, using a redox system containing an azodicarboxylate such as diethyl azodicarboxylate or diisopropyl azodicarboxylate and a phosphine catalyst such as triphenylphosphine.

If desired, pharmaceutically acceptable salts of the compounds of formula I can be prepared by contacting the free acid or base form of a compound of formula I with a pharmaceutically acceptable base or acid.

Certain of the intermediates described herein are believed to be novel and accordingly, such compounds are provided as further aspects of the invention including, for example, compound 9″, depicted without chirality:

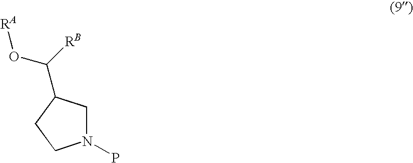

where P represents an amino-protecting group, particularly t-butoxycarbonyl (BOC). In one embodiment of the invention, compounds of the invention can be prepared by deprotecting compound 9″, to provide a compound of formula I, or a salt thereof. In one particular embodiment, such unprotected compounds have the formula of compounds 9 or 9′:

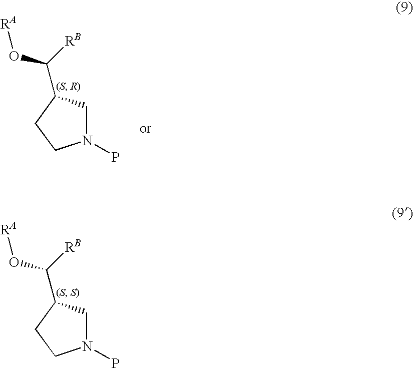

Further details regarding specific reaction conditions and other procedures for preparing representative compounds of the invention or intermediates thereof are described in the Examples set forth herein.

### Utility

Compounds of the invention possess serotonin and norepinephrine reuptake inhibitory activity. Thus, these compounds have therapeutic utility as combined serotonin and norepinephrine reuptake inhibitors (SNRIs). In one embodiment, compounds of the invention possess equal or approximately equal serotonin reuptake inhibitory activity and norepinephrine reuptake inhibitory activity.

The inhibition constant (Kᵢ) of a compound is the concentration of ligand in a radioligand binding inhibition assay that would occupy 50% of the transporters if no radioligand were present. Kᵢ values can be determined from radioligand binding studies with ³H-nisoxetine (for the norepinephrine transporter, NET) and ³H-citalopram (for the serotonin transporter, SERT), as described in Assay 1. These Kᵢ values are derived from IC₅₀ values in the binding assay using the Cheng-Prusoff equation and the K_{d} of the radioligand (Cheng & Prusoff (1973) Biochem. Pharmacol. 22(23):3099-3108). Functional IC₅₀ values can be determined in the functional inhibition of uptake assays described in Assay 2. These IC₅₀ values can be converted to Kᵢ values using the Cheng-Prusoff equation and the Kᵢ of the transmitter for the transporter. It is noted however, that the uptake assay conditions described in Assay 2 are such that the IC₅₀ values are very close to the Kᵢ values, should a mathematical conversion be desired, since the neurotransmitter concentration (5-HT or NE) used in the assay is well below its Kₘ for the respective transporter.

One measure of the affinity of a compound for SERT or NET is the inhibitory constant (pKᵢ) for binding to the transporter. The pKᵢ value is the negative logarithm to base 10 of the Kᵢ. In one embodiment, compounds of the invention have a pKᵢ at SERT or at NET greater than or equal to 5.0, and in another embodiment, have a pKᵢ at SERT and at NET greater than or equal to 5.0. In one particular embodiment, compounds of the invention have a pKᵢ at SERT or at NET greater than or equal to 7.0, and in another embodiment, have a pKᵢ at SERT and at NET greater than or equal to 7.0. In still another embodiment, compounds of the invention have a pKᵢ at SERT or at NET greater than or equal to 8.0, and in another embodiment, have a pKᵢ at SERT and at NET greater than or equal to 8.0. Such values can be determined by techniques that are well know in the art, as well as in the assays described herein.

In one embodiment, compounds of the invention exhibit a SERT Kᵢ/NET Kᵢ in the range of 0.1 to 100; in another embodiment, a SERT Kᵢ/NET Kᵢ in the range of 0.3 to 100; and in still another embodiment, a SERT Kᵢ/NET Kᵢ in the range of 0.3 to 10.

Another measure of serotonin and norepinephrine reuptake inhibition is the pIC₅₀ value. In one embodiment, compounds of interest have a serotonin reuptake inhibition pIC₅₀ value of greater than or equal to 7.0 and a norepinephrine reuptake inhibition pIC₅₀ value of greater than or equal to 7.0; and in another embodiment, compounds of interest have a serotonin reuptake inhibition pIC₅₀ value of greater than or equal to 7.5 and a norepinephrine reuptake inhibition pIC₅₀ value of greater than or equal to 7.5. In one particular embodiment, the compounds have a serotonin reuptake inhibition pIC₅₀ value of greater than or equal to 8.0 and a norepinephrine reuptake inhibition pIC₅₀ value of greater than or equal to 8.0. In one particular embodiment, the compounds of the invention have balanced pIC₅₀ values.

In another embodiment, compounds of the invention are selective for inhibition of SERT and NET over the dopamine transporter (DAT). For example in this embodiment, compounds of particular interest are those that exhibit a binding affinity for SERT and NET that is at least 5 times higher than the binding affinity for DAT, or that is at least 10 times higher than for DAT, or at least 20 or 30 times higher than for DAT. In another embodiment, the compounds do not exhibit significant DAT inhibition. In still another embodiment, the compounds exhibit less than 50% inhibition of DAT activity when measured at a concentration of 794 nM. Under the assay conditions used, a compound which exhibits ≦50% inhibition would have an estimated pKᵢ value at DAT of ≦6.1.

In still another embodiment, compounds of the invention possess dopamine reuptake inhibitory activity as well as SERT and NET activity. For example in this embodiment, compounds of particular interest are those that exhibit a pKᵢ at SERT and NET greater than or equal to 8.0, and a pKᵢ at DAT greater than or equal to 7.0.

It is noted that in some cases, compounds of the invention may possess either weak serotonin reuptake inhibitory activity or weak norepinephrine reuptake inhibitory activity. In these cases, those of ordinary skill in the art will recognize that such compounds still have utility as primarily either a NET inhibitor or a SERT inhibitor, respectively, or will have utility as research tools.

Exemplary assays to determine the serotonin and/or norepinephrine reuptake inhibiting activity of compounds of the invention include by way of illustration and not limitation, assays that measure SERT and NET binding, for example, as described in Assay 1. In addition, it is useful to understand the level of DAT binding and uptake in an assay such as that described in Assay 1. Useful secondary assays include neurotransmitter uptake assays to measure inhibition of serotonin and norepinephrine uptake into cells expressing the respective human or rat recombinant transporter (hSERT or hNET) as described in Assay 2, and ex vivo radioligand binding and neurotransmitter uptake assays that are used to determine the in vivo occupancy of SERT, NET and DAT in tissue as described in Assay 3. Other assays that are useful to evaluate pharmacological properties of test compounds include those listed in Assay 4. Exemplary in vivo assays include the formalin paw test described in Assay 5, which is a reliable predictor of clinical efficacy for the treatment of neuropathic pain, and the spinal nerve ligation model described in Assay 6. The aforementioned assays are useful in determining the therapeutic utility, for example, the neuropathic pain relieving activity, of compounds of the invention. Other properties and utilities of compounds of the invention can be demonstrated using various in vitro and in vivo assays well-known to those skilled in the art.

Compounds of the invention are expected to be useful for the treatment and/or prevention of medical conditions in which the regulation of monoamine transporter function is implicated, in particular those conditions mediated by or responsive to the inhibition of serotonin and norepinephrine reuptake. Thus it is expected that patients suffering from a disease or disorder that is treated by the inhibition of the serotonin and/or the norepinephrine transporter can be treated by administering a therapeutically effective amount of a serotonin and norepinephrine reuptake inhibitor of the invention. Such medical conditions include, by way of example, pain disorders such as neuropathic pain, fibromyalgia, and chronic pain, depressive disorders such as major depression, affective disorders such as an anxiety disorder, attention deficit hyperactivity disorder, cognitive disorders such as dementia, and stress urinary incontinence.

The amount of active agent administered per dose or the total amount administered per day may be predetermined or it may be determined on an individual patient basis by taking into consideration numerous factors, including the nature and severity of the patient's condition, the condition being treated, the age, weight, and general health of the patient, the tolerance of the patient to the active agent, the route of administration, pharmacological considerations such as the activity, efficacy, pharmacokinetics and toxicology profiles of the active agent and any secondary agents being administered, and the like. Treatment of a patient suffering from a disease or medical condition (such as neuropathic pain) can begin with a predetermined dosage or a dosage determined by the treating physician, and will continue for a period of time necessary to prevent, ameliorate, suppress, or alleviate the symptoms of the disease or medical condition. Patients undergoing such treatment will typically be monitored on a routine basis to determine the effectiveness of therapy. For example, in treating neuropathic pain, a measure of the effectiveness of treatment may involve assessment of the patient's quality of life, e.g., improvements in the patient's sleeping patterns, work attendance, ability to exercise and be ambulatory, etc. Pain scales, operating on a point basis, may also be used to help evaluate a patient's pain level. Indicators for the other diseases and conditions described herein, are well-known to those skilled in the art, and are readily available to the treating physician. Continuous monitoring by the physician will ensure that the optimal amount of active agent will be administered at any given time, as well as facilitating the determination of the duration of treatment. This is of particular value when secondary agents are also being administered, as their selection, dosage, and duration of therapy may also require adjustment. In this way, the treatment regimen and dosing schedule can be adjusted over the course of therapy so that the lowest amount of active agent that exhibits the desired effectiveness is administered and, further, that administration is continued only so long as is necessary to successfully treat the disease or medical condition.

### Pain Disorders

SNRIs have been shown to have a beneficial effect on pain such as painful diabetic neuropathy (duloxetine, Goldstein et al. (2005) Pain 116:109-118; venlafaxine, Rowbotham et al. (2004) Pain 110:697-706), fibromyalgia (duloxetine, Russell et al (2008) Pain 136(3):432-444; milnacipran, Vitton et al. (2004) Human Psychopharmacology 19:S27-S35), and migraine (venlafaxine, Ozyalcin et al. (2005) Headache 45(2):144-152). Thus, one embodiment of the invention relates to a method for treating a pain disorder, comprising administering to a patient a therapeutically effective amount of a compound of the invention. Typically, the therapeutically effective amount will be the amount that is sufficient to relieve the pain. Exemplary pain disorders include, by way of illustration, acute pain, persistent pain, chronic pain, inflammatory pain, and neuropathic pain. More specifically, these include pain associated with or caused by: arthritis; back pain including chronic low back pain; cancer, including tumor related pain (e.g. bone pain, headache, facial pain or visceral pain) and pain associated with cancer therapy (e.g. post-chemotherapy syndrome, chronic post-surgical pain syndrome and post-radiation syndrome); carpal tunnel syndrome; fibromyalgia; headaches including chronic tension headaches; inflammation associated with polymyalgia, rheumatoid arthritis and osteoarthritis; migraine; neuropathic pain including complex regional pain syndrome; overall pain; post-operative pain; shoulder pain; central pain syndromes, including post-stroke pain, and pain associated with spinal cord injuries and multiple sclerosis; phantom limb pain; pain associated with Parkinson's disease; and visceral pain (e.g., irritable bowel syndrome). Of particular interest is the treatment of neuropathic pain, which includes diabetic peripheral neuropathy (DPN), HIV-related neuropathy, post-herpetic neuralgia (PHN), and chemotherapy-induced peripheral neuropathy. When used to treat pain disorders such as neuropathic pain, compounds of the invention may be administered in combination with other therapeutic agents, including anticonvulsants, antidepressants, NSAIDs, muscle relaxants, opioid agonists, selective serotonin reuptake inhibitors, sodium channel blockers, and sympatholytics. Exemplary compounds within these classes are described herein.

### Depressive Disorders

Another embodiment of the invention relates to a method of treating a depressive disorder, comprising administering to a patient a therapeutically effective amount of a compound of the invention. Typically, the therapeutically effective amount will be the amount that is sufficient to alleviate depression and provide a sense of general well-being. Exemplary depressive disorders include, by way of illustration and not limitation: depression associated with Alzheimer's disease, bipolar disorder, cancer, child abuse, infertility, Parkinson's disease, postmyocardial infarction, and psychosis; dysthymia; grumpy or irritable old man syndrome; induced depression; major depression; pediatric depression; postmenopausal depression; post partum depression; recurrent depression; single episode depression; and subsyndromal symptomatic depression. Of particular interest is the treatment of major depression. When used to treat depressive disorders, compounds of the invention may be administered in combination with other therapeutic agents, including antidepressants and dual serotonin-norepinephrine reuptake inhibitors. Exemplary compounds within these classes are described herein.

### Affective Disorders

Another embodiment of the invention relates to a method of treating an affective disorder, comprising administering to a patient a therapeutically effective amount of a compound of the invention. Exemplary affective disorders include, by way of illustration and not limitation: anxiety disorders such as general anxiety disorder; avoidant personality disorder; eating disorders such as anorexia nervosa, bulimia nervosa and obesity; obsessive compulsive disorder; panic disorder; personality disorders such as avoidant personality disorder and attention deficit hyperactivity disorder (ADHD); post-traumatic stress syndrome; phobias such as agoraphobia, as well as simple and other specific phobias, and social phobia; premenstrual syndrome; psychotic disorders, such as schizophrenia and mania; seasonal affective disorder; sexual dysfunction, including premature ejaculation, male impotence, and female sexual dysfunction such as female sexual arousal disorder; social anxiety disorder; and substance abuse disorders, including chemical dependencies such as addictions to alcohol, benzodiazepines, cocaine, heroin, nicotine and phenobarbital, as well as withdrawal syndromes that may arise from these dependencies. When used to treat affective disorders, compounds of the invention may be administered in combination with other therapeutic agents, including antidepressants. Exemplary compounds within these classes are described herein.

Atomoxetine, which is 10-fold NET selective, is approved for attention deficit hyperactivity disorder (ADHD) therapy, and clinical studies have shown that the SNRI, venlafaxine, can also have a beneficial effect in treating ADHD (Mukaddes et al. (2002) Eur. Neuropsychopharm. 12(Supp 3):421). Thus, the compounds of the invention are also expected to be useful in methods for treating attention deficit hyperactivity disorder by administering to a patient a therapeutically effective amount of a compound of the invention. When used to treat depression, compounds of the invention may be administered in combination with other therapeutic agents, including antidepressants. Exemplary compounds within these classes are described herein.

### Cognitive Disorders

Another embodiment of the invention relates to a method of treating a cognitive disorder, comprising administering to a patient a therapeutically effective amount of a compound of the invention. Exemplary cognitive disorders include, by way of illustration and not limitation: dementia, which includes degenerative dementia (e g , Alzheimer's disease, Creutzfeldt-Jakob disease, Huntingdon's chorea, Parkinson's disease, Pick's disease, and senile dementia), vascular dementia (e.g., multi-infarct dementia), and dementia associated with intracranial space occupying lesions, trauma, infections and related conditions (including HIV infection), metabolism, toxins, anoxia and vitamin deficiency; and mild cognitive impairment associated with aging, such as age associated memory impairment, amnesiac disorder and age-related cognitive decline. When used to treat cognitive disorders, compounds of the invention may be administered in combination with other therapeutic agents, including anti-Alzheimer's agents and anti-Parkinson's agents. Exemplary compounds within these classes are described herein.

### Other Disorders

SNRIs have also been shown to be effective for the treatment of stress urinary incontinence (Dmochowski (2003) Journal of Urology 170(4): 1259-1263). Thus, another embodiment of the invention relates to a method for treating stress urinary incontinence, comprising administering to a patient a therapeutically effective amount of a compound of the invention. When used to treat stress urinary incontinence, compounds of the invention may be administered in combination with other therapeutic agents, including anticonvulsants. Exemplary compounds within these classes are described herein.

Duloxetine, an SNRI, is undergoing clinical trials for evaluating its efficacy in treating chronic fatigue syndrome, and has recently been shown to be effective in treating fibromyalgia (Russell et al. (2008) Pain 136(3):432-444). The compounds of the invention, due to their ability to inhibit SERT and NET, are also expected to have this utility, and another embodiment of the invention relates to a method for treating chronic fatigue syndrome, comprising administering to a patient a therapeutically effective amount of a compound of the invention.

Sibutramine, a norepinephrine and dopamine reuptake inhibitor, has been shown to be useful in treating obesity (Wirth et al. (2001) JAMA 286(11):1331-1339). The compounds of the invention, due to their ability to inhibit NET, are also expected to have this utility, and another embodiment of the invention relates to a method for treating obesity, comprising administering to a patient a therapeutically effective amount of a compound of the invention.

Desvenlafaxine, an SNRI, has been shown to relieve vasomotor symptoms associated with menopause (Deecher et al. (2007) Endocrinology 148(3):1376-1383). The compounds of the invention, due to their ability to inhibit SERT and NET, are also expected to have this utility, and another embodiment of the invention relates to a method for treating vasomotor symptoms associated with menopause, comprising administering to a patient a therapeutically effective amount of a compound of the invention.

### Research Tools

Since compounds of the invention possess both serotonin reuptake inhibition activity and norepinephrine reuptake inhibition activity, such compounds are also useful as research tools for investigating or studying biological systems or samples having serotonin or norepinephrine transporters. Any suitable biological system or sample having serotonin and/or norepinephrine transporters may be employed in such studies which may be conducted either in vitro or in vivo. Representative biological systems or samples suitable for such studies include, but are not limited to, cells, cellular extracts, plasma membranes, tissue samples, isolated organs, mammals (such as mice, rats, guinea pigs, rabbits, dogs, pigs, humans, and so forth), and the like, with mammals being of particular interest. In one particular embodiment of the invention, serotonin reuptake in a mammal is inhibited by administering a serotonin reuptake-inhibiting amount of a compound of the invention. In another particular embodiment, norepinephrine reuptake in a mammal is inhibited by administering a norepinephrine reuptake-inhibiting amount of a compound of the invention. Compounds of the invention can also be used as research tools by conducting biological assays using such compounds.

When used as a research tool, a biological system or sample comprising a serotonin transporter and/or a norepinephrine transporter is typically contacted with a serotonin reuptake-inhibiting or norepinephrine reuptake-inhibiting amount of a compound of the invention. After the biological system or sample is exposed to the compound, the effects of inhibiting serotonin reuptake and/or norepinephrine reuptake are determined using conventional procedures and equipment. Exposure encompasses contacting cells or tissue with the compound, administering the compound to a mammal, for example by i.p. or i.v. administration, and so forth. This determining step may comprise measuring a response, i.e., a quantitative analysis or may comprise an observation, i.e., a qualitative analysis. Measuring a response involves, for example, determining the effects of the compound on the biological system or sample using conventional procedures and equipment, such as serotonin and norepinephrine reuptake assays. The assay results can be used to determine the activity level as well as the amount of compound necessary to achieve the desired result, i.e., a serotonin reuptake-inhibiting and a norepinephrine reuptake-inhibiting amount.

Additionally, compounds of the invention can be used as research tools for evaluating other chemical compounds, and thus are also useful in screening assays to discover, for example, new compounds having both serotonin reuptake-inhibiting activity and norepinephrine reuptake-inhibiting activity. In this manner, a compound of the invention is used as a standard in an assay to allow comparison of the results obtained with a test compound and with compounds of the invention to identify those test compounds that have about equal or superior reuptake-inhibiting activity, if any. For example, reuptake data for a test compound or a group of test compounds is compared to the reuptake data for a compound of the invention to identify those test compounds that have the desired properties, e.g., test compounds having reuptake-inhibiting activity about equal or superior to a compound of the invention, if any. This aspect of the invention includes, as separate embodiments, both the generation of comparison data (using the appropriate assays) and the analysis of the test data to identify test compounds of interest. Thus, a test compound can be evaluated in a biological assay, by a method comprising the steps of: (a) conducting a biological assay with a test compound to provide a first assay value; (b) conducting the biological assay with a compound of the invention to provide a second assay value; wherein step (a) is conducted either before, after or concurrently with step (b); and (c) comparing the first assay value from step (a) with the second assay value from step (b). Exemplary biological assays include serotonin and norepinephrine reuptake assays.

### Pharmaceutical Compositions and Formulations

Compounds of the invention are typically administered to a patient in the form of a pharmaceutical composition or formulation. Such pharmaceutical compositions may be administered to the patient by any acceptable route of administration including, but not limited to, oral, rectal, vaginal, nasal, inhaled, topical (including transdermal) and parenteral modes of administration. Further, the compounds of the invention may be administered, for example orally, in multiple doses per day (e.g. two, three times or four times daily), in a single daily dose, in a twice-daily dose, in a single weekly dose, and so forth. It will be understood that any form of the compounds of the invention, (i.e., free base, pharmaceutically acceptable salt, solvate, etc.) that is suitable for the particular mode of administration can be used in the pharmaceutical compositions discussed herein.

Accordingly, in one embodiment, the invention relates to a pharmaceutical composition comprising a pharmaceutically acceptable carrier and a compound of the invention. The compositions may contain other therapeutic and/or formulating agents if desired. When discussing compositions, the “compound of the invention” may also be referred to herein as the “active agent,” to distinguish it from other components of the formulation, such as the carrier. Thus, it is understood that the term “active agent” includes compounds of formula I as well as pharmaceutically acceptable salts and solvates of that compound.

The pharmaceutical compositions of the invention typically contain a therapeutically effective amount of a compound of the invention. Those skilled in the art will recognize, however, that a pharmaceutical composition may contain more than a therapeutically effective amount, i.e., bulk compositions, or less than a therapeutically effective amount, i.e., individual unit doses designed for multiple administration to achieve a therapeutically effective amount. Typically, the composition will contain from about 0.01-95 wt % of active agent, including, from about 0.01-30 wt %, such as from about 0.01-10 wt %, with the actual amount depending upon the formulation itself, the route of administration, the frequency of dosing, and so forth. In one embodiment, a composition suitable for an oral dosage form, for example, may contain about 5-70 wt %, or from about 10-60 wt % of active agent.

Any conventional carrier or excipient may be used in the pharmaceutical compositions of the invention. The choice of a particular carrier or excipient, or combinations of carriers or excipients, will depend on the mode of administration being used to treat a particular patient or type of medical condition or disease state. In this regard, the preparation of a suitable composition for a particular mode of administration is well within the scope of those skilled in the pharmaceutical arts. Additionally, carriers or excipients used in such compositions are commercially available. By way of further illustration, conventional formulation techniques are described in Remington: The Science and Practice of Pharmacy, 20^{th} Edition, Lippincott Williams & White, Baltimore, Md. (2000); and Ansel et al., Pharmaceutical Dosage Forms and Drug Delivery Systems, 7^{th} Edition, Lippincott Williams & White, Baltimore, Md. (1999).

Representative examples of materials which can serve as pharmaceutically acceptable carriers include, but are not limited to, the following: sugars, such as lactose, glucose and sucrose; starches, such as corn starch and potato starch; cellulose, such as microcrystalline cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; compressed propellant gases, such as chlorofluorocarbons and hydrofluorocarbons; and other non-toxic compatible substances employed in pharmaceutical compositions.

Pharmaceutical compositions are typically prepared by thoroughly and intimately mixing or blending the active agent with a pharmaceutically acceptable carrier and one or more optional ingredients. The resulting uniformly blended mixture may then be shaped or loaded into tablets, capsules, pills, canisters, cartridges, dispensers, and the like, using conventional procedures and equipment.

In one embodiment, the pharmaceutical compositions are suitable for oral administration. One exemplary dosing regimen would be an oral dosage form administered once or twice daily. Suitable compositions for oral administration may be in the form of capsules, tablets, pills, lozenges, cachets, dragees, powders, granules; solutions or suspensions in an aqueous or non-aqueous liquid; oil-in-water or water-in-oil liquid emulsions; elixirs or syrups; and the like; each containing a predetermined amount of the active agent.

When intended for oral administration in a solid dosage form (i.e., as capsules, tablets, pills, and the like), the composition will typically comprise the active agent and one or more pharmaceutically acceptable carriers, such as sodium citrate or dicalcium phosphate. Solid dosage forms may also comprise: fillers or extenders, such as starches, microcrystalline cellulose, lactose, sucrose, glucose, mannitol, and/or silicic acid; binders, such as carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and/or sodium carbonate; solution retarding agents, such as paraffin; absorption accelerators, such as quaternary ammonium compounds; wetting agents, such as cetyl alcohol and/or glycerol monostearate; absorbents, such as kaolin and/or bentonite clay; lubricants, such as talc, calcium stearate, magnesium stearate, solid polyethylene glycols, sodium lauryl sulfate, and/or mixtures thereof; coloring agents; and buffering agents.

Release agents, wetting agents, coating agents, sweetening, flavoring and perfuming agents, preservatives and antioxidants may also be present in the pharmaceutical compositions. Exemplary coating agents for tablets, capsules, pills and like, include those used for enteric coatings, such as cellulose acetate phthalate, polyvinyl acetate phthalate, hydroxypropyl methylcellulose phthalate, methacrylic acid-methacrylic acid ester copolymers, cellulose acetate trimellitate, carboxymethyl ethyl cellulose, hydroxypropyl methyl cellulose acetate succinate, and the like. Examples of pharmaceutically acceptable antioxidants include: water-soluble antioxidants, such as ascorbic acid, cysteine hydrochloride, sodium bisulfate, sodium metabisulfate, sodium sulfite and the like; oil-soluble antioxidants, such as ascorbyl palmitate, butylated hydroxyanisole, butylated hydroxytoluene, lecithin, propyl gallate, alpha-tocopherol, and the like; and metal-chelating agents, such as citric acid, ethylenediamine tetraacetic acid, sorbitol, tartaric acid, phosphoric acid, and the like.

Compositions may also be formulated to provide slow or controlled release of the active agent using, by way of example, hydroxypropyl methyl cellulose in varying proportions or other polymer matrices, liposomes and/or microspheres. In addition, the pharmaceutical compositions of the invention may contain opacifying agents and may be formulated so that they release the active agent only, or preferentially, in a certain portion of the gastrointestinal tract, optionally, in a delayed manner. Examples of embedding compositions which can be used include polymeric substances and waxes. The active agent can also be in micro-encapsulated form, if appropriate, with one or more of the above-described excipients.

Suitable liquid dosage forms for oral administration include, by way of illustration, pharmaceutically acceptable emulsions, microemulsions, solutions, suspensions, syrups and elixirs. Liquid dosage forms typically comprise the active agent and an inert diluent, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (e.g., cottonseed, groundnut, corn, germ, olive, castor and sesame oils), glycerol, tetrahydrofuryl alcohol, polyethylene glycols and fatty acid esters of sorbitan, and mixtures thereof. Suspensions may contain suspending agents such as, for example, ethoxylated isostearyl alcohols, polyoxyethylene sorbitol and sorbitan esters, microcrystalline cellulose, aluminium metahydroxide, bentonite, agar-agar and tragacanth, and mixtures thereof.

When intended for oral administration, the pharmaceutical compositions of the invention may be packaged in a unit dosage form. The term “unit dosage form” refers to a physically discrete unit suitable for dosing a patient, i.e., each unit containing a predetermined quantity of the active agent calculated to produce the desired therapeutic effect either alone or in combination with one or more additional units. For example, such unit dosage forms may be capsules, tablets, pills, and the like.

In another embodiment, the compositions of the invention are suitable for inhaled administration, and will typically be in the form of an aerosol or a powder. Such compositions are generally administered using well-known delivery devices, such as a nebulizer, dry powder, or metered-dose inhaler. Nebulizer devices produce a stream of high velocity air that causes the composition to spray as a mist that is carried into a patient's respiratory tract. An exemplary nebulizer formulation comprises the active agent dissolved in a carrier to form a solution, or micronized and combined with a carrier to form a suspension of micronized particles of respirable size. Dry powder inhalers administer the active agent as a free-flowing powder that is dispersed in a patient's air-stream during inspiration. An exemplary dry powder formulation comprises the active agent dry-blended with an excipient such as lactose, starch, mannitol, dextrose, polylactic acid, polylactide-co-glycolide, and combinations thereof. Metered-dose inhalers discharge a measured amount of the active agent using compressed propellant gas. An exemplary metered-dose formulation comprises a solution or suspension of the active agent in a liquefied propellant, such as a chlorofluorocarbon or hydrofluoroalkane. Optional components of such formulations include co-solvents, such as ethanol or pentane, and surfactants, such as sorbitan trioleate, oleic acid, lecithin, and glycerin. Such compositions are typically prepared by adding chilled or pressurized hydrofluoroalkane to a suitable container containing the active agent, ethanol (if present) and the surfactant (if present). To prepare a suspension, the active agent is micronized and then combined with the propellant. Alternatively, a suspension formulation can be prepared by spray drying a coating of surfactant on micronized particles of the active agent. The formulation is then loaded into an aerosol canister, which forms a portion of the inhaler.

Compounds of the invention can also be administered parenterally (e.g., by subcutaneous, intravenous, intramuscular, or intraperitoneal injection). For such administration, the active agent is provided in a sterile solution, suspension, or emulsion. Exemplary solvents for preparing such formulations include water, saline, low molecular weight alcohols such as propylene glycol, polyethylene glycol, oils, gelatin, fatty acid esters such as ethyl oleate, and the like. A typical parenteral formulation is a sterile pH 4-7 aqueous solution of the active agent. Parenteral formulations may also contain one or more solubilizers, stabilizers, preservatives, wetting agents, emulsifiers, and dispersing agents. These formulations may be rendered sterile by use of a sterile injectable medium, a sterilizing agent, filtration, irradiation, or heat.

Compounds of the invention can also be administered transdermally using known transdermal delivery systems and excipients. For example, the compound can be admixed with permeation enhancers, such as propylene glycol, polyethylene glycol monolaurate, azacycloalkan-2-ones and the like, and incorporated into a patch or similar delivery system. Additional excipients including gelling agents, emulsifiers and buffers, may be used in such transdermal compositions if desired.

If desired, compounds of the invention may be administered in combination with one or more other therapeutic agents. Thus, in one embodiment, compositions of the invention may optionally contain other drugs that are co-administered with a compound of the invention. For example, the composition may further comprise one or more drugs (also referred to as “secondary agents(s)”) selected from the group of anti-Alzheimer's agents, anticonvulsants (antiepileptics), antidepressants, anti-Parkinson's agents, dual serotonin-norepinephrine reuptake inhibitors (SNRIs), non-steroidal anti-inflammatory agents (NSAIDs), norepinephrine reuptake inhibitors, opioid agonists (opioid analgesics), selective serotonin reuptake inhibitors, sodium channel blockers, sympatholytics, and combinations thereof. Numerous examples of such therapeutic agents are well known in the art, and examples are described herein. By combining a compound of the invention with a secondary agent, triple therapy can be achieved, i.e., serotonin reuptake inhibitory activity, norepinephrine reuptake inhibitory activity, and activity associated with the secondary agent (e.g., antidepressant activity), using only two active components. Since pharmaceutical compositions containing two active components are typically easier to formulate than compositions containing three active components, such two-component compositions provide a significant advantage over compositions containing three active components. Accordingly, in yet another aspect of the invention, a pharmaceutical composition comprises a compound of the invention, a second active agent, and a pharmaceutically acceptable carrier. Third, fourth etc. active agents may also be included in the composition. In combination therapy, the amount of compound of the invention that is administered, as well as the amount of secondary agents, may be less than the amount typically administered in monotherapy.

A compound of the invention may be either physically mixed with the second active agent to form a composition containing both agents; or each agent may be present in separate and distinct compositions which are administered to the patient simultaneously or sequentially. For example, a compound of the invention can be combined with a second active agent using conventional procedures and equipment to form a combination of active agents comprising a compound of the invention and a second active agent. Additionally, the active agents may be combined with a pharmaceutically acceptable carrier to form a pharmaceutical composition comprising a compound of the invention, a second active agent and a pharmaceutically acceptable carrier. In this embodiment, the components of the composition are typically mixed or blended to create a physical mixture. The physical mixture is then administered in a therapeutically effective amount using any of the routes described herein.

Alternatively, the active agents may remain separate and distinct before administration to the patient. In this embodiment, the agents are not physically mixed together before administration but are administered simultaneously or at separate times as separate compositions. Such compositions can be packaged separately or may be packaged together in a kit. When administered at separate times, the secondary agent will typically be administered less than 24 hours after administration of the compound of the invention, ranging anywhere from concurrent with administration of the compound of the invention to about 24 hours post-dose. This is also referred to as sequential administration. Thus, a compound of the invention can be orally administered simultaneously or sequentially with another active agent using two tablets, with one tablet for each active agent, where sequential may mean being administered immediately after administration of the compound of the invention or at some predetermined time later (e.g., one hour later or three hours later). Alternatively, the combination may be administered by different routes of administration, i.e., one orally and the other by inhalation.

In one embodiment, the kit comprises a first dosage form comprising a compound of the invention and at least one additional dosage form comprising one or more of the secondary agents set forth herein, in quantities sufficient to carry out the methods of the invention. The first dosage form and the second (or third, etc,) dosage form together comprise a therapeutically effective amount of active agents for the treatment or prevention of a disease or medical condition in a patient.

Secondary agent(s), when included, are present in a therapeutically effective amount. i.e., are typically administered in an amount that produces a therapeutically beneficial effect when co-administered with a compound of the invention. The secondary agent can be in the form of a pharmaceutically acceptable salt, solvate, optically pure stereoisomer, and so forth. Thus, secondary agents listed below are intended to include all such forms, and are commercially available or can be prepared using conventional procedures and reagents.

Representative anti-Alzheimer's agents include, but are not limited to: donepezil, galantamine, memantine, rivastigmine, selegiline, tacrine, and combinations thereof.

Representative anticonvulsants (antiepileptics) include, but are not limited to: acetazolamide, albutoin, 4-amino-3-hydroxybutyric acid, beclamide, carbamazepine, cinromide, clomethiazole, clonazepam, diazepam, dimethadione, eterobarb, ethadione, ethosuximide, ethotoin, felbamate, fosphenytoin, gabapentin, lacosamide, lamotrigine, lorazepam, magnesium bromide, magnesium sulfate, mephenytoin, mephobarbital, methsuximide, midazolam, nitrazepam, oxazepam, oxcarbazepine, paramethadione, phenacemide, pheneturide, phenobarbital, phensuximide, phenytoin, potassium bromide, pregabalin, primidone, progabide, sodium bromide, sodium valproate, sulthiame, tiagabine, topiramate, trimethadione, valproic acid, valpromide, vigabatrin, zonisamide, and combinations thereof. In a particular embodiment, the anticonvulsant is selected from carbamazepine, gabapentin, pregabalin, and combinations thereof.

Representative antidepressants include, but are not limited to: adinazolam, amitriptyline, clomipramine, desipramine, dothiepin (e.g., dothiepin hydrochloride), doxepin, imipramine, lofepramine, mirtazapine, nortriptyline, protriptyline, trimipramine, venlafaxine, zimelidine, and combinations thereof.

Representative anti-Parkinson's agents include, but are not limited to: amantadine, apomorphine, benztropine, bromocriptine, carbidopa, diphenhydramine, entacapone, levodopa, pergolide, pramipexole, ropinirole, selegiline, tolcapone, trihexyphenidyl, and combinations thereof.

Representative dual serotonin-norepinephrine reuptake inhibitors (SNRIs) include, but are not limited to: bicifadine, desvenlafaxine, duloxetine, milnacipran, nefazodone, venlafaxine, and combinations thereof.

Representative non-steroidal anti-inflammatory agents (NSAIDs) include, but are not limited to: acemetacin, acetaminophen, acetyl salicylic acid, alclofenac, alminoprofen, amfenac, amiprilose, amoxiprin, anirolac, apazone, azapropazone, benorilate, benoxaprofen, bezpiperylon, broperamole, bucloxic acid, carprofen, clidanac, diclofenac, diflunisal, diftalone, enolicam, etodolac, etoricoxib, fenbufen, fenclofenac, fenclozic acid, fenoprofen, fentiazac, feprazone, flufenamic acid, flufenisal, fluprofen, flurbiprofen, furofenac, ibufenac, ibuprofen, indomethacin, indoprofen, isoxepac, isoxicam, ketoprofen, ketorolac, lofemizole, lornoxicam, meclofenamate, meclofenamic acid, mefenamic acid, meloxicam, mesalamine, miroprofen, mofebutazone, nabumetone, naproxen, niflumic acid, nimesulide, nitroflurbiprofen, olsalazine, oxaprozin, oxpinac, oxyphenbutazone, phenylbutazone, piroxicam, pirprofen, pranoprofen, salsalate, sudoxicam, sulfasalazine, sulindac, suprofen, tenoxicam, tiopinac, tiaprofenic acid, tioxaprofen, tolfenamic acid, tolmetin, triflumidate, zidometacin, zomepirac, and combinations thereof. In a particular embodiment, the NSAID is selected from etodolac, flurbiprofen, ibuprofen, indomethacin, ketoprofen, ketorolac, meloxicam, naproxen, oxaprozin, piroxicam, and combinations thereof. In a particular embodiment, the NSAID is selected from ibuprofen, indomethacin, nabumetone, naproxen (for example, naproxen sodium), and combinations thereof.

Representative muscle relaxants include, but are not limited to: carisoprodol, chlorzoxazone, cyclobenzaprine, diflunisal, metaxalone, methocarbamol, and combinations thereof.

Representative norepinephrine reuptake inhibitors include, but are not limited to: atomoxetine, buproprion and the buproprion metabolite hydroxybuproprion, maprotiline, reboxetine (for example, (S,S)-reboxetine), viloxazine, and combinations thereof. In a particular embodiment, the norepinephrine reuptake inhibitor is selected from atomoxetine, reboxetine, and combinations thereof.

Representative opioid agonists (opioid analgesics) include, but are not limited to: buprenorphine, butorphanol, codeine, dihydrocodeine, fentanyl, hydrocodone, hydromorphone, levallorphan, levorphanol, meperidine, methadone, morphine, nalbuphine, nalmefene, nalorphine, naloxone, naltrexone, nalorphine, oxycodone, oxymorphone, pentazocine, propoxyphene, tramadol, and combinations thereof. In certain embodiments, the opioid agonist is selected from codeine, dihydrocodeine, hydrocodone, hydromorphone, morphine, oxycodone, oxymorphone, tramadol, and combinations thereof.

Representative selective serotonin reuptake inhibitors (SSRIs) include, but are not limited to: citalopram and the citalopram metabolite desmethylcitalopram, dapoxetine, escitalopram (e.g., escitalopram oxalate), fluoxetine and the fluoxetine desmethyl metabolite norfluoxetine, fluvoxamine (e.g., fluvoxamine maleate), paroxetine, sertraline and the sertraline metabolite demethylsertraline, and combinations thereof. In certain embodiments, the SSRI is selected from citalopram, paroxetine, sertraline, and combinations thereof.

Representative sodium channel blockers include, but are not limited to: carbamazepine, fosphenytoin, lamotrignine, lidocaine, mexiletine, oxcarbazepine, phenytoin, and combinations thereof.

Representative sympatholytics include, but are not limited to: atenolol, clonidine, doxazosin, guanethidine, guanfacine, modafinil, phentolamine, prazosin, reserpine, tolazoline (e.g., tolazoline hydrochloride), tamsulosin, and combinations thereof.

The following formulations illustrate representative pharmaceutical compositions of the present invention:

### Exemplary Hard Gelatin Capsules for Oral Administration

A compound of the invention (50 g), spray-dried lactose (440 g) and magnesium stearate (10 g) are thoroughly blended. The resulting composition is then loaded into hard gelatin capsules (500 mg of composition per capsule).

Alternately, a compound of the invention (20 mg) is thoroughly blended with starch (89 mg), microcrystalline cellulose (89 mg) and magnesium stearate (2 mg). The mixture is then passed through a No. 45 mesh U.S. sieve and loaded into a hard gelatin capsule (200 mg of composition per capsule).

### Exemplary Gelatin Capsule Formulation for Oral Administration

A compound of the invention (100 mg) is thoroughly blended with polyoxyethylene sorbitan monooleate (50 mg) and starch powder (250 mg). The mixture is then loaded into a gelatin capsule (400 mg of composition per capsule).

Alternately, a compound of the invention (40 mg) is thoroughly blended with microcrystalline cellulose (Avicel PH 103; 259.2 mg) and magnesium stearate (0.8 mg). The mixture is then loaded into a gelatin capsule (Size #1, White, Opaque) (300 mg of composition per capsule).

### Exemplary Tablet Formulation for Oral Administration

A compound of the invention (10 mg), starch (45 mg) and microcrystalline cellulose (35 mg) are passed through a No. 20 mesh U.S. sieve and mixed thoroughly. The granules so produced are dried at 50-60° C. and passed through a No. 16 mesh U.S. sieve. A solution of polyvinylpyrrolidone (4 mg as a 10% solution in sterile water) is mixed with sodium carboxymethyl starch (4.5 mg), magnesium stearate (0.5 mg), and talc (1 mg), and this mixture is then passed through a No. 16 mesh U.S. sieve. The sodium carboxymethyl starch, magnesium stearate and talc are then added to the granules. After mixing, the mixture is compressed on a tablet machine to afford a tablet weighing 100 mg.

Alternately, a compound of the invention (250 mg) is thoroughly blended with microcrystalline cellulose (400 mg), silicon dioxide fumed (10 mg), and stearic acid (5 mg). The mixture is then compressed to form tablets (665 mg of composition per tablet).

Alternately, a compound of the invention (400 mg) is thoroughly blended with cornstarch (50 mg), croscarmellose sodium (25 mg), lactose (120 mg), and magnesium stearate (5 mg). The mixture is then compressed to form a single-scored tablet (600 mg of compositions per tablet).

### Exemplary Suspension Formulation for Oral Administration

The following ingredients are mixed to form a suspension containing 100 mg of active agent per 10 mL of suspension:

| Ingredients | Amount | |
|---|---|---|
| Compound of the invention | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum ® K (magnesium aluminum silicate) | 1.0 | g |
| Flavoring | 0.035 | mL |
| Colorings | 0.5 | mg |
| Distilled water | q.s. to 100 | mL |

### Exemplary Injectable Formulation for Administration by Injection

A compound of the invention (0.2 g) is blended with 0.4 M sodium acetate buffer solution (2.0 mL). The pH of the resulting solution is adjusted to pH 4 using 0.5 N aqueous hydrochloric acid or 0.5 N aqueous sodium hydroxide, as necessary, and then sufficient water for injection is added to provide a total volume of 20 mL. The mixture is then filtered through a sterile filter (0.22 micron) to provide a sterile solution suitable for administration by injection.

### Exemplary Compositions for Administration by Inhalation

A compound of the invention (0.2 mg) is micronized and then blended with lactose (25 mg). This blended mixture is then loaded into a gelatin inhalation cartridge. The contents of the cartridge are administered using a dry powder inhaler, for example.

Alternately, a micronized compound of the invention (10 g) is dispersed in a solution prepared by dissolving lecithin (0.2 g) in demineralized water (200 mL). The resulting suspension is spray dried and then micronized to form a micronized composition comprising particles having a mean diameter less than about 1.5 μm. The micronized composition is then loaded into metered-dose inhaler cartridges containing pressurized 1,1,1,2-tetrafluoroethane in an amount sufficient to provide about 10 μg to about 500 μg of the compound of the invention per dose when administered by the inhaler.

Alternately, a compound of the invention (25 mg) is dissolved in citrate buffered (pH 5) isotonic saline (125 mL). The mixture is stirred and sonicated until the compound is dissolved. The pH of the solution is checked and adjusted, if necessary, to pH 5 by slowly adding aqueous 1N sodium hydroxide. The solution is administered using a nebulizer device that provides about 10 μg to about 500 μg of the compound of the invention per dose.

### Examples

The following Preparations and Examples are provided to illustrate specific embodiments of the invention. These specific embodiments, however, are not intended to limit the scope of the invention in any way unless specifically indicated.

The following abbreviations have the following meanings unless otherwise indicated and any other abbreviations used herein and not defined have their standard meaning.
AcOH acetic acid
ACN acetonitrile
Boc t-butoxycarbonyl
Bn benzyl
BSA bovine serum albumin
DCM dichloromethane (i.e., methylene chloride)
DMEM Dulbecco's Modified Eagle's Medium
DMF N,N-dimethylformamide
DMSO dimethylsulfoxide
EDTA ethylenediaminetetraacetic acid
Et ethyl
EtOAc ethyl acetate
EtOH ethanol
FBS fetal bovine serum
hDAT human dopamine transporter
iPro isopropyl
HEPES 4-(2-hydroxyethyl)-1-piperazineethanesulfonic acid
hNET human norepinephrine transporter
hSERT human serotonin transporter
5-HT 5-hydroxytryptamine
Me methyl
MeOH methanol
PBS phosphate buffered saline
Ph phenyl
TEMPO 2,2,6,6-tetramethyl-1-piperidinyloxy, free radical
TFA trifluoroacetic acid
THF tetrahydrofuran

Any other abbreviations used herein but not defined have their standard, generally accepted meaning. Unless noted otherwise, all materials, such as reagents, starting materials and solvents, were purchased from commercial suppliers (such as Sigma-Aldrich, Fluka Riedel-de Haën, and the like) and were used without further purification.

In the following figures, the two chiral centers are identified by the * and ** symbols. When describing the stereochemistry, the carbon atom indicated by the * symbol is designated first. Thus, an “SR” designation represents a compound having the (S) configuration at the carbon atom indicated by the * symbol and having the (R) configuration at the ** carbon atom. The same hold true for racemic mixtures. For example, an “RS/SR” designation represents a racemic mixture of (R,S) compounds and (S,R) compounds, i.e., a mixture of compounds having the (R) configuration at the * carbon atom and the (S) configuration at the ** carbon atom and compounds having the (S) configuration at the * carbon atom and the (R) configuration at the ** carbon atom.

Note that the * chiral center is known to be S. However, the ** chiral center is not known unambiguously and was designated R or S based upon the first elution peak by reverse phase HPLC from the mixture of diastereomeric protected alcohols. The first eluting peak was designated R at the ** chiral center and the second eluting peak was designated S at the ** chiral center.

### Preparation 1

### (S)-3-formylpyrrolidine-1-carboxylic Acid t-Butyl Ester

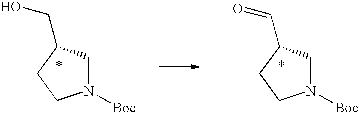

To a solution of (S)-3-hydroxymethylpyrrolidine-1-carboxylic acid t-butyl ester (7.4 g, 37 mmol) in DCM (74 mL, 1.2 mol) was added TEMPO (100 mg, 0.7 mmol) and potassium bromide (200 mg, 2 mmol). This mixture was cooled to 0° C. and vigorously stirred as a pre-chilled (at 0° C.) 1:1 mixture of 0.7 M of NaOCl in water (78 mL, 55 mmol) and a saturated, aqueous NaHCO₃ solution (75 mL) was added dropwise over a period of 10 minutes. The resultant mixture was extracted with DCM (3×100 mL). The combined organic layers were washed with water (2×100 mL), then saturated aqueous NaCl (1×100 mL). The organic layer was dried over anhydrous Na₂SO₄, filtered and concentrated in vacuo to yield (S)-3-formylpyrrolidine-1-carboxylic acid t-butyl ester, which was used without further purification (5.8 g).

### Preparation 2

### (S)-3-[(S)-(5-Fluoropyridin-3-yl)hydroxymethyl]pyrrolidine-1-carboxylic Acid t-Butyl Ester and (S)-3-[(R)-(5-Fluoropyridin-3-yl)hydroxymethyl]pyrrolidine-1-carboxylic Acid t-Butyl Ester

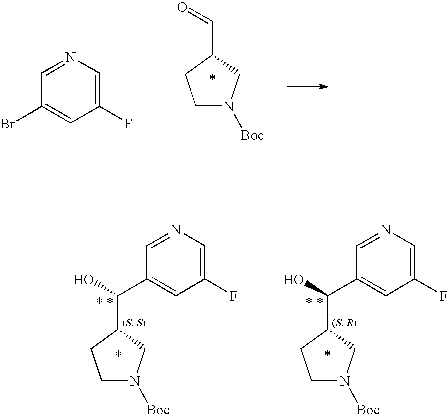

A solution of 3-bromo-5-fluoropyridine (1 g, 5.7 mmol) in THF (10 mL) was placed in a flask that had been purged with and maintained under nitrogen. A solution of butyl magnesium chloride (720 mg, 6.2 mmol) in THF (3.1 mL) was added dropwise with stirring, and the resulting solution was stirred for 3 hours at 25° C. A solution of (S)-3-formylpyrrolidine-1-carboxylic acid t-butyl ester (1.1 g, 5.3 mmol) in THF (3 mL) was added dropwise with stirring at 0° C. in 0.5 hour, and the resulting solution was stirred for 2 hours at 25° C. The reaction was then quenched by the addition of 10 mL of water/ice. The resulting solution was extracted with EtOAc (2×10 mL) and the combined organic layers were dried over anhydrous Na₂SO₄. The solids were filtered out and the filtrate was concentrated in vacuo. The residue was applied onto a silica gel column with EtOAc/petroleum ether (1:2) to yield (S)-3-[(5-fluoropyridin-3-yl)hydroxymethyl]pyrrolidine-1-carboxylic acid t-butyl ester as a crude yellow oil (500 mg).

### (S,S) Product

Several lots of the crude product were combined (7.5 g) and purified by chiral preparative HPLC with the following conditions (Gilson Pre-HPLC): column 20 mm×250 mm, 5 micron particle size (Daicel Chemical Ind., Ltd.); mobile phase hexanes/EtOH=90/10; detector UV 254 nm. This resulted in obtaining 2.1 g (28% yield) of (S)-3-[(S)-(5-fluoropyridin-3-yl)hydroxymethyl]pyrrolidine-1-carboxylic acid t-butyl ester as a slight yellow solid:

ES m/z: [M-Boc-H₂O+H]⁺ calcd for C₁₀H₁₂FN₂, 179; found 179.

¹H-NMR (400 MHz, CDCl₃): δ(ppm)=8.41 (2H, s), 7.54 (1H, m), 4.65 (1H, d, J=8.2 Hz), 3.61 (1H, m), 3.46 (1H,s), 3.35 (1H, m), 3.33 (1H, m), 2.51 (1H, s), 1.63 (2H, m), 1.45 (9h, s).

The (S,S) product was analyzed for chiral purity and found to be 99.78% ee.

### (S,R) Product

Several lots of the crude product were combined (4.5 g) and purified by chiral preparative HPLC with the following conditions (Gilson Pre-HPLC): column 20 mm×250 mm, 20 micron particle size (Daicel Chemical Ind., Ltd.); mobile phase hexanes/isopropanol=92/8; detector UV 254 nm. This resulted in obtaining 2.0 g (45% yield) of (S)-3-[(R)-(5-fluoropyridin-3-yl)hydroxymethyl]pyrrolidine-1-carboxylic acid t-butyl ester as a white solid:

ES m/z: [M-Boc-H₂O+H]⁺ calcd for C₁₀H₁₂FN₂, 179; found 179.

¹H-NMR (400 MHz, CDCl₃): δ(ppm)=8.41(2H, d, J=7.2 Hz), 7.47 (1H, d, J=8.8 Hz), 4.69 (1H, d, J=7.6 Hz), 3.51 (1H, s), 3.27 (2H, s), 3.05 (1H, s), 2.51 (1H, m), 2.14 (1H, s), 1.90 (1H, m), 1.43 (9H, s).

The (S,R) product was analyzed for chiral purity and found to be 99.81% ee.

### Example 1

### 3-[(R)-(4-Chloro-2-methoxyphenoxy)-(S)-pyrrolidin-3-yl-methyl]-5-fluoropyridine

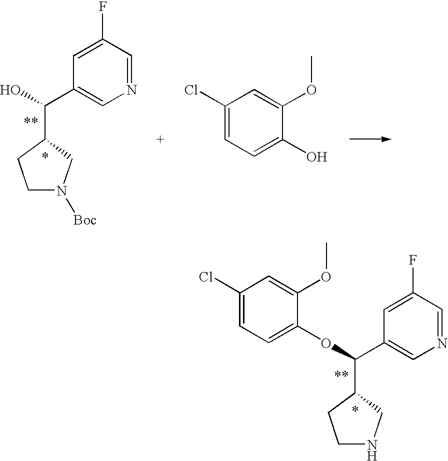

(S)-3-[(S)-(5-Fluoropyridin-3-yl)hydroxymethyl]pyrrolidine-1-carboxylic acid t-butyl ester (40 mg, 0.1 mmol) and triphenylphosphine (41.0 mg, 0.156 mmol) in THF (84.6 μL, 1.0 mmol) was combined, and to the solution was added 4-chloro-2-methoxy-phenol (21.8 μL). The vial was then sonicated for several minutes for proper mixing. While sonicating, diisopropyl azodicarboxylate (30.8 μL, 156 μmol) was added dropwise to the mixture over a couple minutes and then sonicated for 15 minutes. The mixture was concentrated, redissolved in 1.25 M of HCl in EtOH (0.8 mL, 1 mmol) and stirred overnight. The concentrated mixture was redissolved in a 1:1 AcOH:H₂O solution and purified by preparative HPLC to yield the title compound as a TFA salt (50.2 mg; 96% purity). MS m/z: [M+H]⁺ calcd for C₁₇H₁₈ClFN₂O₂, 337.10; found 337.0.

### Example 2

Following the procedures described in the example above, the method depicted in Scheme C, or the Mitsunobu coupling reaction, and substituting the appropriate starting materials and reagents, compounds 1 to 66, having the following formula were also prepared as TFA salts:

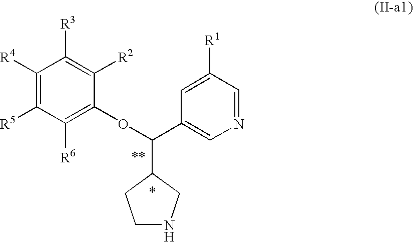

where R¹ is Cl, F or H

The following compounds have R¹ as Cl:

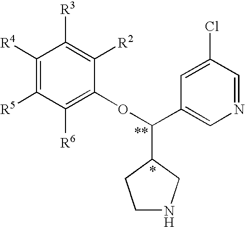

| | | | | | | | | | MS m/z: | |
|---|---|---|---|---|---|---|---|---|---|---|
| | | | | | | | | | [M + H]⁺ | |
| # | * | ** | R² | R³ | R⁴ | R⁵ | R⁶ | Formula | calcd | found |
| 1 | S | R | Cl | H | Cl | Cl | H | C₁₆H₁₄Cl₄N₂O | 390.99 | 391.0 |
| 2 | S | R | Cl | F | Cl | H | H | C₁₆H₁₄Cl₃FN₂O | 375.02 | 375.7 |
| 3 | S | R | Cl | F | H | F | Cl | C₁₆H₁₃Cl₃F₂N₂O | 393.01 | 393.0 |
| 4 | S | S | Cl | F | H | F | Cl | C₁₆H₁₃Cl₃F₂N₂O | 393.01 | 393.0 |
| 5 | S | R | Bn | H | Cl | H | H | C₂₃H₂₂Cl₂N₂O | 413.11 | 413.0 |
| 6 | S | R | Cl | F | H | H | F | C₁₆H₁₄Cl₂F₂N₂O | 359.05 | 359.0 |
| 7 | S | S | Cl | F | H | H | F | C₁₆H₁₄Cl₂F₂N₂O | 359.05 | 359.0 |
| 8 | S | R | —COMe | Cl | Cl | H | H | C₁₈H₁₇Cl₃N₂O₂ | 399.04 | 399.0 |
| 9 | S | R | —COMe | H | F | H | F | C₁₈H₁₇ClF₂N₂O₂ | 367.09 | 367.0 |
| 10 | S | R | Et | H | F | H | F | C₁₈H₁₉ClF₂N₂O | 353.12 | 353.0 |
| 11 | S | R | Me | H | Cl | H | H | C₁₇H₁₈Cl₂N₂O | 337.08 | 337.0 |
| 12 | S | R | OMe | H | H | H | H | C₁₇H₁₉ClN₂O₂ | 319.11 | 319.0 |
| 13 | S | S | OMe | H | H | H | H | C₁₇H₁₉ClN₂O₂ | 319.11 | 319.0 |
| 14 | S | R | OMe | H | Cl | F | H | C₁₇H₁₇Cl₂FN₂O₂ | 371.07 | 371.0 |
| 15 | S | R | H | Cl | H | Cl | H | C₁₆H₁₅Cl₃N₂O | 357.03 | 357.0 |
| 16 | S | S | H | Cl | H | Cl | H | C₁₆H₁₅Cl₃N₂O | 357.03 | 357.0 |
| 17 | S | R | —CF₃ | H | Cl | H | H | C₁₇H₁₅Cl₂F₃N₂O | 391.05 | 391.0 |
| 18 | S | R | Cl | H | Cl | H | H | C₁₆H₁₅Cl₃N₂O | 357.03 | 357.0 |
| 19 | S | R | —C(O)—OCH₃ | H | Cl | H | H | C₁₈H₁₈Cl₂N₂O₃ | 381.07 | 381.0 |
| 20 | S | R | Cl | H | F | H | H | C₁₆H₁₅Cl₂FN₂O | 341.05 | 341.0 |
| 21 | S | R | Et | H | F | H | H | C₁₈H₂₀ClFN₂O | 335.12 | 335.0 |
| 22 | S | S | OMe | H | Cl | H | H | C₁₇H₁₈Cl₂N₂O₂ | 353.07 | 353.0 |
| 23 | S | R | Cl | H | Me | H | H | C₁₇H₁₈Cl₂N₂O | 337.08 | 337.0 |
| 24 | S | R | Cl | H | Ph | H | H | C₂₂H₂₀Cl₂N₂O | 399.10 | 399.0 |
| 25 | S | R | F | H | Cl | H | F | C₁₆H₁₄Cl₂F₂N₂O | 359.05 | 359.0 |
| 26 | S | R | Cl | H | F | H | Cl | C₁₆H₁₄Cl₃FN₂O | 375.02 | 375.0 |
| 27 | S | R | Cl | H | Cl | H | F | C₁₆H₁₄Cl₃FN₂O | 375.02 | 375.0 |
| 28 | S | R | Cl | H | Me | H | Cl | C₁₇H₁₇Cl₃N₂O | 371.04 | 371.0 |
| 29 | S | R | Me | H | Cl | H | Me | C₁₈H₂₀Cl₂N₂O | 351.10 | 351.0 |
| 30 | S | R | Cl | H | Cl | H | Me | C₁₇H₁₇Cl₃N₂O | 371.04 | 371.0 |
| 31 | S | S | F | H | Cl | H | H | C₁₆H₁₅Cl₂FN₂O | 341.05 | 341.0 |
| 32 | S | R | F | H | F | H | H | C₁₆H₁₅ClF₂N₂O | 325.08 | 325.0 |
| 33 | S | R | F | H | F | H | F | C₁₆H₁4ClF₃N₂O | 343.07 | 343.0 |

```
  1. 3-Chloro-5-[(R)-(S)-pyrrolidin-3-y1-(2,4,5-trichlorophenoxy)methyl]pyridine
  2. 3-Chloro-5-[(R)-(2,4-dichloro-3-fluorophenoxy)-(S)-pyrrolidin-3-yl-methyl]-pyridine
  3. 3-Chloro-5-[(R)-(2,6-dichloro-3,5-difluorophenoxy)-(S)-pyrrolidin-3-yl-methyl]-pyridine
  4. 3-Chloro-5-[(S)-(2,6-dichloro-3,5-difluorophenoxy)-(S)-pyrrolidin-3-yl-methyl]-pyridine
  5. 3-[(R)-(2-Benzyl-4-chlorophenoxy)-(S)-pyrrolidin-3-yl-methyl]-5-chloropyridine
  6. 3-Chloro-5-[(R)-(2-chloro-3,6-difluorophenoxy)-(S)-pyrrolidin-3-yl-methyl]-pyridine
  7. 3-Chloro-5-[(S)-(2-chloro-3,6-difluorophenoxy)-(S)-pyrrolidin-3-yl-methyl]-pyridine
  8. 1-{2,3-Dichloro-6-[(R)-(5-chloropyridin-3-yl)-(S)-pyrrolidin-3-yl-methoxy]-phenyl}ethanone
  9. 1-{2-[(R)-(5-Chloropyridin-3-yl)-(S)-pyrrolidin-3-yl-methoxy]-3,5-difluoro-phenyl}ethanone
10. 3-Chloro-5-[(R)-(2-ethyl-4,6-difluorophenoxy)-(S)-pyrrolidin-3-yl-methyl]pyridine
11. 3-Chloro-5-[(R)-(4-chloro-2-methylphenoxy)-(S)-pyrrolidin-3-yl-methyl]pyridine
12. 3-Chloro-5-[(R)-(2-methoxyphenoxy)-(S)-pyrrolidin-3-yl-methyl]pyridine
13. 3-Chloro-5-[(S)-(2-methoxyphenoxy)-(S)-pyrrolidin-3-yl-methyl]pyridine
14. 3-Chloro-5-[(R)-(4-chloro-5-fluoro-2-methoxyphenoxy)-(S)-pyrrolidin-3-yl-methyl]pyridine
15. 3-Chloro-5-[(R)-(3,5-dichlorophenoxy)-(S)-pyrrolidin-3-yl-methyl]pyridine
16. 3-Chloro-5-[(S)-(3,5-dichlorophenoxy)-(S)-pyrrolidin-3-yl-methyl]pyridine
17. 3-Chloro-5-[(R)-(4-chloro-2-trifluoromethylphenoxy)-(S)-pyrrolidin-3-yl-methyl]-pyridine
18. 3-Chloro-5-[(R)-(2,4-dichlorophenoxy)-(S)-pyrrolidin-3-yl-methyl]pyridine
19. 5-Chloro-2-[(R)-(5-chloropyridin-3-yl)-(S)-pyrrolidin-3-yl-methoxy]-benzoic acid methyl ester
20. 3-Chloro-5-[(R)-(2-chloro-4-fluorophenoxy)-(S)-pyrrolidin-3-yl-methyl]pyridine
21. 3-Chloro-5-[(R)-(2-ethyl-4-fluorophenoxy)-(S)-pyrrolidin-3-yl-methyl]pyridine
22. 3-Chloro-5-[(R)-(4-chloro-2-methoxyphenoxy)-(S)-pyrrolidin-3-yl-methyl]pyridine
23. 3-Chloro-5-[(R)-(2-chloro-4-methylphenoxy)-(S)-pyrrolidin-3-yl-methyl]pyridine
24. 3-Chloro-5-[(R)-(3-chlorobiphenyl-4-yloxy)-(S)-pyrrolidin-3-yl-methyl]pyridine
25. 3-Chloro-5-[(R)-(4-chloro-2,6-difluorophenoxy)-(S)-pyrrolidin-3-yl-methy1]-pyridine
26. 3-Chloro-5-[(R)-(2,6-dichloro-4-fluorophenoxy)-(S)-pyrrolidin-3-yl-methy1]-pyridine
27. 3-Chloro-5-[(R)-(2,4-dichloro-6-fluorophenoxy)-(S)-pyrrolidin-3-yl-methy1]-pyridine
28. 3-Chloro-5-[(R)-(2,6-dichloro-4-methylphenoxy)-(S)-pyrrolidin-3-yl-methy1]-pyridine
29. 3-Chloro-5-[(R)-(4-chloro-2,6-dimethylphenoxy)-(S)-pyrrolidin-3-yl-methy1]-pyridine
30. 3-Chloro-5-[(R)-(2,4-dichloro-6-methylphenoxy)-(S)-pyrrolidin-3-yl-methy1]-pyridine
31. 3-Chloro-5-[(R)-(4-chloro-2-fluorophenoxy)-(S)-pyrrolidin-3-yl-methyl]pyridine
32. 3-Chloro-5-[(R)-(2,4-difluorophenoxy)-(S)-pyrrolidin-3-yl-methyl]pyridine
33. 3-Chloro-5-[(R)-(S)-pyrrolidin-3-yl-(2,4,6-trifluorophenoxy)methyl]pyridine
```

The following compounds have R¹ as F:

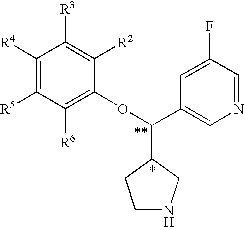

| | | | | | | | | | MS m/z: | |
|---|---|---|---|---|---|---|---|---|---|---|
| | | | | | | | | | [M + H]⁺ | |
| # | * | ** | R² | R³ | R⁴ | R⁵ | R⁶ | Formula | calcd | found |
| 34 | S | R | Cl | H | Cl | Cl | H | C₁₆H₁₄Cl₃FN₂O | 375.02 | 375.0 |
| 35 | S | R | F | H | F | H | F | C₁₆H₁₄F₄N₂O | 327.10 | 327.0 |
| 36 | S | R | Cl | H | Cl | H | H | C₁₆H₁₅Cl₂FN₂O | 341.05 | 341.0 |
| 37 | S | R | Cl | F | Cl | H | H | C₁₆H₁₄Cl₂F₂N₂O | 359.05 | 359.0 |
| 38 | S | R | Cl | H | Cl | H | F | C₁₆H₁₄Cl₂F₂N₂O | 359.05 | 359.0 |
| 39 | S | R | Cl | H | Cl | H | Me | C₁₇H₁₇Cl₂FN₂O | 355.07 | 355.0 |
| 40 | S | R | F | H | F | H | H | C₁₆H₁₅F₃N₂O | 309.11 | 309.0 |
| 41 | S | R | Cl | F | H | F | Cl | C₁₆H₁₃C1₂F₃N₂O | 377.04 | 377.0 |
| 42 | S | S | Cl | F | H | F | Cl | C₁₆H₁₃C1₂F₃N₂O | 377.04 | 377.0 |
| 43 | S | R | Cl | H | F | H | Cl | C₁₆H₁₄Cl₂F₂N₂O | 359.05 | 359.0 |
| 44 | S | R | Cl | H | Me | H | Cl | C₁₇H₁₇Cl₂FN₂O | 355.07 | 355.0 |
| 45 | S | R | F | H | Cl | H | F | C₁₆H₁₄ClF₃N₂O | 343.07 | 343.0 |
| 46 | S | R | Me | H | Cl | H | Me | C₁₈H₂₀ClFN₂O | 335.12 | 335.0 |
| 47 | S | R | Bn | H | Cl | H | H | C₂₃H₂₂ClFN₂O | 397.14 | 397.0 |
| 48 | S | R | CF₃ | H | Cl | H | H | C₁₇H₁₅ClF₄N₂O | 375.08 | 375.0 |
| 49 | S | R | Cl | F | H | H | F | C₁₆H₁₄ClF₃N₂O | 343.07 | 343.0 |
| 50 | S | S | Cl | F | H | H | F | C₁₆H₁₄ClF₃N₂O | 343.07 | 343.0 |
| 51 | S | R | Cl | H | F | H | H | C₁₆H₁₅ClF₂N₂O | 325.08 | 325.0 |
| 52 | S | R | Cl | H | Me | H | H | C₁₇H₁₈ClFN₂O | 321.11 | 321.0 |
| 53 | S | R | Cl | H | Ph | H | H | C₂₂H₂₀ClFN₂O | 383.13 | 383.0 |
| 54 | S | R | —COMe | Cl | Cl | H | H | C₁₈H₁₇Cl₂FN₂O₂ | 383.07 | 383.0 |
| 55 | S | R | —COMe | H | F | H | F | C₁₈H₁₇F₃N₂O₂ | 351.12 | 351.2 |
| 56 | S | R | —COOMe | H | Cl | H | H | C₁₈H₁₈ClFN₂O₃ | 365.10 | 365.0 |
| 57 | S | R | Et | H | F | H | F | C₁₈H₁₉F₃N₂O | 337.15 | 337.0 |
| 58 | S | R | Et | H | F | H | H | C₁₈H₂₀F₂N₂O | 319.15 | 319.2 |
| 59 | S | R | F | H | Cl | H | H | C₁₆H₁₅ClF₂N₂O | 325.08 | 325.0 |
| 60 | S | R | Me | H | Cl | H | H | C₁₇H₁₈ClFN₂O | 321.11 | 321.0 |
| 61 | S | S | Me | H | Cl | H | H | C₁₇H₁₈ClFN₂O | 321.11 | 321.0 |
| 62 | S | R | OMe | H | H | H | H | C₁₇H₁₉FN₂O₂ | 303.14 | 303.0 |
| 63 | S | S | OMe | H | H | H | H | C₁₇H₁₉FN₂O₂ | 303.14 | 303.2 |
| 64 | S | R | OMe | H | Cl | F | H | C₁₇H₁₇ClF₂N₂O₂ | 355.09 | 355.0 |
| 65 | S | R | H | Cl | H | Cl | H | C₁₆H₁₅Cl₂FN₂O | 341.05 | 341.0 |
| 66 | S | S | H | Cl | H | Cl | H | C₁₆H₁₅Cl₂FN₂O | 341.05 | 341.0 |

34. 3-Fluoro-5-[(R)-(S)-pyrrolidin-3-yl-(2,4,5-trichlorophenoxy)-methyl]pyridine
35. 3-Fluoro-5-[(R)-(S)-pyrrolidin-3-yl-(2,4,6-trifluorophenoxy)-methyl]pyridine
36. 3-[(R)-(2,4-Dichlorophenoxy)-(S)-pyrrolidin-3-yl-methyl]-5-fluoropyridine
37. 3-[(R)-(2,4-Dichloro-3-fluorophenoxy)-(S)-pyrrolidin-3-yl-methyl]-5-fluoropyridine
38. 3-[(R)-(2,4-Dichloro-6-fluorophenoxy)-(S)-pyrrolidin-3-yl-methyl]-5-fluoropyridine
39. 3-[(R)-(2,4-Dichloro-6-methylphenoxy)-(S)-pyrrolidin-3-yl-methyl]-5-fluoropyridine
40. 3-[(R)-(2,4-Difluorophenoxy)-(S)-pyrrolidin-3-yl-methyl]-5-fluoropyridine
41. 3-[(R)-(2,6-Dichloro-3,5-difluorophenoxy)-(S)-pyrrolidin-3-yl-methyl]-5-fluoropyridine
42. 3-[(S)-(2,6-Dichloro-3,5-difluorophenoxy)-(S)-pyrrolidin-3-yl-methyl]-5-fluoropyridine
43. 3-[(R)-(2,6-Dichloro-4-fluorophenoxy)-(S)-pyrrolidin-3-yl-methyl]-5-fluoropyridine
44. 3-[(R)-(2,6-Dichloro-4-methylphenoxy)-(S)-pyrrolidin-3-yl-methyl]-5-fluoropyridine
45. 3-[(R)-(4-Chloro-2,6-difluorophenoxy)-(S)-pyrrolidin-3-yl-methyl]-5-fluoropyridine
46. 3-[(R)-(4-Chloro-2,6-dimethylphenoxy)-(S)-pyrrolidin-3-yl-methy1]-5-fluoropyridine
47. 3-[(R)-(2-Benzy1-4-chlorophenoxy)-(S)-pyrrolidin-3-yl-methy1]-5-fluoropyridine
48. 3-[(R)-(4-Chloro-2-trifluoromethylphenoxy)-(S)-pyrrolidin-3-yl-methyl]-5-fluoropyridine
49. 3-[(R)-(2-Chloro-3,6-difluorophenoxy)-(S)-pyrrolidin-3-yl-methyl]-5-fluoropyridine
50. 3-[(S)-(2-Chloro-3,6-difluorophenoxy)-(S)-pyrrolidin-3-yl-methy1]-5-fluoropyridine
51. 3-[(R)-(2-Chloro-4-fluorophenoxy)-(S)-pyrrolidin-3-yl-methyl]-5-fluoropyridine
52. 3-[(R)-(2-Chloro-4-methylphenoxy)-(S)-pyrrolidin-3-yl-methyl]-5-fluoropyridine
53. 3-[(R)-(3-Chlorobiphenyl-4-yloxy)-(S)-pyrrolidin-3-yl-methyl]-5-fluoropyridine
54. 1-{2,3-Dichloro-6-[(R)-(5-fluoropyridin-3-yl)-(S)-pyrrolidin-3-yl-methoxy]phenyl}ethanone
55. 1-{3,5-Difluoro-2-[(R)-(5-fluoropyridin-3-yl)-(S)-pyrrolidin-3-yl-methoxy]phenyl}ethanone
56. 5-Chloro-2-[(R)-(5-fluoropyridin-3-yl)-(S)-pyrrolidin-3-yl-methoxy]benzoic acid methyl ester
57. 3-[(R)-(2-Ethy1-4,6-difluorophenoxy)-(S)-pyrrolidin-3-yl-methyl]-5-fluoropyridine
58. 3-[(R)-(2-Ethy1-4-fluorophenoxy)-(S)-pyrrolidin-3-yl-methyl]-5-fluoropyridine
59. 3-[(R)-(4-Chloro-2-fluorophenoxy)-(S)-pyrrolidin-3-yl-methyl]-5-fluoropyridine
60. 3-[(R)-(4-Chloro-2-methylphenoxy)-(S)-pyrrolidin-3-yl-methyl]-5-fluoropyridine
61. 3-[(S)-(4-Chloro-2-methylphenoxy)-(S)-pyrrolidin-3-yl-methyl]-5-fluoropyridine
62. 3-Fluoro-5-[(R)-(2-methoxyphenoxy)-(S)-pyrrolidin-3-yl-methyl]pyridine
63. 3-Fluoro-5-[(S)-(2-methoxyphenoxy)-(S)-pyrrolidin-3-yl-methyl]pyridine
64. 3-[(R)-(4-Chloro-5-fluoro-2-methoxyphenoxy)-(S)-pyrrolidin-3-yl-methyl]-5-fluoropyridine
65. 3-[(R)-(3,5-Dichlorophenoxy)-(S)-pyrrolidin-3-yl-methyl]-5-fluoropyridine
66. 3-[(S)-(3,5-Dichlorophenoxy)-(S)-pyrrolidin-3-yl-methyl]-5-fluoropyridine

The following compounds have R¹ as H:

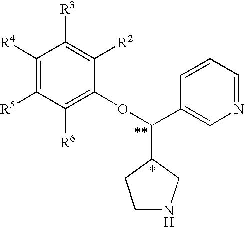

| | | | | | | | | | MS m/z: | |
|---|---|---|---|---|---|---|---|---|---|---|
| | | | | | | | | | [M + H]⁺ | |
| # | * | ** | R² | R³ | R⁴ | R⁵ | R⁶ | Formula | calcd | found |
| 67 | S | R | Cl | Cl | H | H | Cl | C₁₆H₁₅Cl₃N₂O | 357.03 | 357.0 |
| 68 | S | R | F | H | F | H | F | C₁₆H₁₅F₃N₂O | 309.11 | 309.0 |
| 69 | S | S | F | H | F | H | F | C₁₆H₁₅F₃N₂O | 309.11 | 309.0 |
| 70 | S | R | F | H | F | H | H | C₁₆H₁₆F₂N₂O | 291.12 | 291.0 |
| 71 | S | S | F | H | F | H | H | C₁₆H₁₆F₂N₂O | 291.12 | 291.0 |
| 72 | S | R | Cl | H | H | H | Cl | C₁₆H₁₆Cl₂N₂O | 323.06 | 323.2 |
| 73 | S | S | Cl | H | H | H | Cl | C₁₆H₁₆Cl₂N₂O | 323.06 | 323.2 |
| 74 | SR/SS | | Cl | Me | H | H | Cl | C₁₇H₁₈Cl₂N₂O | 337.08 | 337.2 |
| 75 | S | R | Cl | Me | H | H | Cl | C₁₇H₁₈Cl₂N₂O | 337.08 | 337.2 |
| 76 | 5 | R | F | H | Cl | H | F | C₁₆H₁₅ClF₂N₂O | 325.08 | 325.0 |
| 77 | S | R | Cl | Me | H | H | F | C₁₇H₁₈ClFN₂O | 321.11 | 321.0 |
| 78 | S | R | Et | H | F | H | F | C₁₈H₂₀F₂N₂O | 319.15 | 319.2 |
| 79 | S | R | F | Me | H | H | Cl | C₁₇H₁₈ClFN₂O | 321.11 | 321.0 |
| 80 | S | R | Me | H | Cl | H | H | C₁₇H₁₉ClN₂O | 303.12 | 303.0 |
| 81 | SR/SS | | OMe | H | H | H | H | C₁₇H₂₀N₂O₂ | 285.15 | 285.2 |
| 82 | S | R | OMe | H | H | H | H | C₁₇H₂₀N₂O₂ | 285.15 | 285.2 |

67. 3-[(R)-(S)-Pyrrolidin-3-yl-(2,3,6-trichlorophenoxy)methyl]pyridine
68. 3-[(R)-(S)-Pyrrolidin-3-yl-(2,4,6-trifluorophenoxy)methyl]pyridine
69. 3-[(S)-(S)-Pyrrolidin-3-yl-(2,4,6-trifluorophenoxy)methyl]pyridine
70. 3-[(R)-(2,4-Difluorophenoxy)-(S)-pyrrolidin-3-yl-methyl]pyridine
71. 3-[(S)-(2,4-Difluorophenoxy)-(S)-pyrrolidin-3-yl-methyl]pyridine
72. 3-[(R)-(2,6-Dichlorophenoxy)-(S)-pyrrolidin-3-yl-methyl]pyridine
73. 3-[(S)-(2,6-Dichlorophenoxy)-(S)-pyrrolidin-3-yl-methyl]pyridine
74. 3-[(S)-(2,6-Dichloro-3-methylphenoxy)-pyrrolidin-3-yl-methyl]pyridine
75. 3-[(R)-(2,6-Dichloro-3-methylphenoxy)-(S)-pyrrolidin-3-yl-methyl]pyridine
76. 3-[(R)-(4-Chloro-2,6-difluorophenoxy)-(S)-pyrrolidin-3-yl-methyl]pyridine
77. 3-[(R)-(2-Chloro-6-fluoro-3-methylphenoxy)-(S)-pyrrolidin-3-yl-methyl]pyridine
78. 3-[(R)-(2-Ethyl-4,6-difluorophenoxy)-(S)-pyrrolidin-3-yl-methyl]pyridine
79. 3-[(R)-(6-Chloro-2-fluoro-3-methylphenoxy)-(S)-pyrrolidin-3-yl-methyl]pyridine
80. 3-[(R)-(4-Chloro-2-methylphenoxy)-(S)-pyrrolidin-3-yl-methyl]pyridine
81. 3-[(S)-(2-Methoxyphenoxy)-pyrrolidin-3-yl-methyl]pyridine
82. 3-[(R)-(2-Methoxyphenoxy)-(S)-pyrrolidin-3-yl-methyl]pyridine

### Example 3

Following the procedures described in the examples above or in the Schemes, and substituting the appropriate starting materials and reagents, compounds 1 to 7, having the following formula were also prepared as TFA salts:

(II-a3)

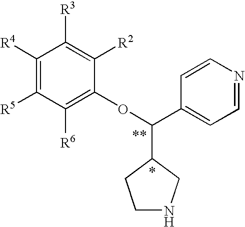

| | | | | | | | | | MS m/z: [M + H]⁺ | |
|---|---|---|---|---|---|---|---|---|---|---|
| # | * | ** | R² | R³ | R⁴ | R⁵ | R⁶ | Formula | calcd | found |
| 1 | S | R | F | H | F | H | F | C₁₆H₁₅F₃N₂O | 309.11 | 309.0 |
| 2 | S | S | F | H | F | H | F | C₁₆H₁₅F₃N₂O | 309.11 | 309.0 |
| 3 | SR/SS | | F | H | F | H | H | C₁₆H₁₆F₂N₂O | 291.12 | 291.0 |
| 4 | S | R | Cl | H | H | H | Cl | C₁₆H₁₆Cl₂N₂O | 323.06 | 323.2 |
| 5 | S | S | Cl | H | H | H | Cl | C₁₆H₁₆Cl₂N₂O | 323.06 | 323.2 |
| 6 | S | R | Cl | Me | H | H | Cl | C₁₇H₁₈Cl₂N₂O | 337.08 | 337.2 |
| 7 | S | S | Cl | Me | H | H | Cl | C₁₆H₁₈Cl₂N₂O | 337.08 | 337.2 |

1 4-[(R)-(S)-Pyrrolidin-3-yl-(2,4,6-trifluorophenoxy)methyl]pyridine
2 4-[(S)-(S)-Pyrrolidin-3-yl-(2,4,6-trifluorophenoxy)methyl]pyridine
3 4-[(S)-(2,4-Difluorophenoxy)-pyrrolidin-3-yl-methyl]pyridine
4 4-[(R)-(2,6-Dichlorophenoxy)-(S)-pyrrolidin-3-yl-methyl]pyridine
5 4-[(S)-(2,6-Dichlorophenoxy)-(S)-pyrrolidin-3-yl-methyl]pyridine
6 4-[(R)-(2,6-Dichloro-3-methylphenoxy)-(S)-pyrrolidin-3-yl-methyl]pyridine
7 4-[(S)-(2,6-Dichloro-3-methylphenoxy)-(S)-pyrrolidin-3-yl-methyl]pyridine

### Example 4

### 2-[((S)-(4-Chloro-2-methylphenoxy)((S)-pyrrolidin-3-yl-methyl]thiazole

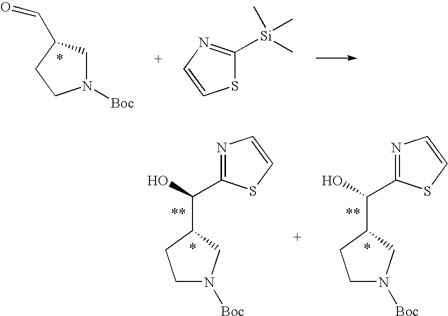

(S)-3-formylpyrrolidine-1-carboxylic acid t-butyl ester (3.1 g, 15.8 mmol) and cesium fluoride (30 mg, 0.2 mmol) in THF (35 mL, 388 mmol) were placed in a flask, cooled at 0° C., under an atmosphere of nitrogen. 2-(Trimethylsilyl)thiazole (3.8 mL, 23.6 mmol) was slowly added, via syringe, over 5 minutes. The mixture was allowed to warm to room temperature and stirred at this temperature for 2 hours, then saturated NH₄Cl (10 mL) and 1M HCl (2 mL) were added to quench the reaction. The mixture was stirred at room temperature for 3 hours, before adding EtOAc (100 mL). The organic layer was collected, dried over anhydrous Na₂SO₄, and the solvent removed by vacuum. No separation of diastereomers was observed by HPLC. The crude product was purified by preparative HPLC (3×2 g crude) by dissolving in 50% AcOH/water (3×4 mL) and purifying by gradient as shown: 10-50% ACN/water (0.05% TFA) over 80 minutes on the 2″ column at 40 mL/min. The fractions were collected and lyophilized to give a mixture of the diastereomers (S)-3-((R)-hydroxythiazol-2-yl-methyl)pyrrolidine-1-carboxylic acid t-butyl ester and (S)-3-((S)-hydroxythiazol-2-yl-methyl)pyrrolidine-1-carboxylic acid t-butyl ester as an oil (3.0 g).

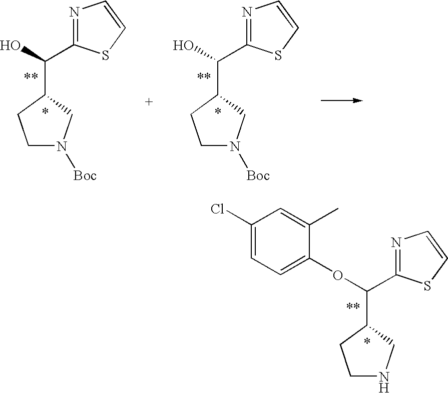

A mixture of the (S)-3-(hydroxylthiazol-2-yl-methyl)pyrrolidine-1-carboxylic acid t-butyl ester diastereomers (205 mg, 721 μmol) was dissolved in THF (0.5 mL, 6 mmol). 4-Chloro-2-methylphenol (154 mg, 1.1 mmol) and triphenylphosphine (208 mg, 793 μmol) were added, and the mixture sonicated for 2 minutes to allow dissolution. Once dissolved diisopropyl azodicarboxylate (156 μL, 0.793 mmol) was slowly added dropwise, via a syringe, while sonicating. The reaction was left to sonicate for 15 minutes, at room temperature. After 15 minutes the reaction was stopped and the solvent was removed by vacuum to leave an oil.

Solid liquid extraction was done as follows. PL-HCO₃ MP-resins were equilibrated with MeOH (1 mL). The oil was dissolved in MeOH (1 mL), transferred to the cartridge and eluted via gravity. The resin was washed with MeOH (4 mL) and the solvent removed. The resulting product was stirred in 1.25 M HCl in EtOH (2 mL, 2 mmol) and stirred overnight, after which the reaction was stopped and the solvent removed. The product was purified by preparative HPLC then the diastereomers were separated to give the title compound (46.9 mg) as a TFA salt. MS m/z: [M+H]⁺ calcd for C₁₅H₁₇ClFN₂OS, 308.83; found 309.2.

### Example 5

Following the procedures described in the examples above or in the Schemes, and substituting the appropriate starting materials and reagents, compounds 1 to 33, having the following formula were also prepared as TFA salts:

(II-b1)

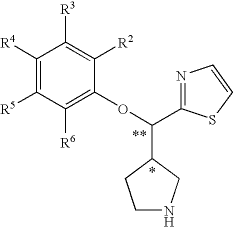

| | | | | | | | | | MS m/z: [M + H]⁺ | |
|---|---|---|---|---|---|---|---|---|---|---|
| # | * | ** | R² | R³ | R⁴ | R⁵ | R⁶ | Formula | calcd | found |
| 1 | S | R | F | F | F | H | H | C₁₄H₁₃F₃N₂OS | 315.07 | 315.0 |
| 2 | S | S | F | F | F | H | H | C₁₄H₁₃F₃N₂OS | 315.07 | 315.0 |
| 3 | S | R | F | F | H | F | F | C₁₄H₁₂F₄N₂OS | 333.06 | 333.0 |
| 4 | S | S | F | F | H | F | F | C₁₄H₁₂F₄N₂OS | 333.06 | 333.0 |
| 5 | SR/SS | | F | H | F | H | F | C₁₄H₁₃F₃N₂OS | 315.07 | 315.0 |
| 6 | S | R | F | H | F | H | F | C₁₄H₁₃F₃N₂OS | 315.07 | 315.0 |
| 7 | S | S | F | H | F | H | F | C₁₄H₁₃F₃N₂OS | 315.07 | 315.0 |
| 8 | S | R | Cl | F | H | F | Cl | C₁₄H₁₂Cl₂F₂N₂OS | 365.00 | 365.0 |
| 9 | S | S | Cl | F | H | F | Cl | C₁₄H₁₂Cl₂F₂N₂OS | 365.00 | 365.0 |
| 10 | S | R | Cl | H | F | H | Cl | C₁₄H₁₃Cl₂FN₂OS | 347.01 | 347.0 |
| 11 | S | S | Cl | H | F | H | Cl | C₁₄H₁₃Cl₂FN₂OS | 347.01 | 347.0 |
| 12 | SR/SS | | Cl | H | Me | H | Cl | C₁₅H₁₆Cl₂N₂OS | 343.04 | 343.0 |
| 13 | S | R | Cl | H | Me | H | Cl | C₁₅H₁₆Cl₂N₂OS | 343.04 | 343.0 |
| 14 | S | S | Cl | H | Me | H | Cl | C₁₅H₁₆Cl₂N₂OS | 343.04 | 343.0 |
| 15 | S | R | Cl | F | H | H | F | C₁₄H₁₃ClF₂N₂OS | 331.04 | 331.0 |
| 16 | S | S | Cl | F | H | H | F | C₁₄H₁₃ClF₂N₂OS | 331.04 | 331.0 |
| 17 | SR/SS | | Cl | Me | H | H | F | C₁₅H₁₆ClFN₂OS | 327.07 | 327.0 |
| 18 | S | R | Cl | Me | H | H | F | C₁₅H₁₆ClFN₂OS | 327.07 | 327.0 |
| 19 | S | S | Cl | H | F | H | H | C₁₄H₁₄ClFN₂OS | 313.05 | 313.2 |
| 20 | SR/SS | | Cl | H | F | H | H | C₁₄H₁₄ClFN₂OS | 313.05 | 313.2 |
| 21 | S | R | Cl | H | F | H | H | C₁₄H₁₄ClFN₂OS | 313.05 | 313.2 |
| 22 | S | R | Me | H | Cl | H | H | C₁₅H₁₇ClN₂OS | 309.08 | 309.0 |
| 23 | S | S | Cl | Me | H | H | F | C₁₅H₁₆ClFN₂OS | 327.07 | 327.0 |
| 24 | S | R | Me | H | H | H | H | C₁₅H₁₈N₂OS | 275.11 | 275.0 |
| 25 | S | S | Me | H | H | H | H | C₁₅H₁₈N₂OS | 275.11 | 275.0 |
| 26 | S | R | H | Cl | H | Cl | H | C₁₄H₁₄Cl₂N₂OS | 329.02 | 329.0 |
| 27 | S | S | H | Cl | H | Cl | H | C₁₄H₁₄Cl₂N₂OS | 329.02 | 329.0 |
| 28 | S | R | F | H | OMe | H | F | C₁₅H₁₆F₂N₂O₂S | 327.09 | 327.0 |
| 29 | SR/SS | | F | H | OMe | H | F | C₁₅H₁₆F₂N₂O₂S | 327.09 | 327.0 |
| 30 | S | R | H | Cl | F | H | H | C₁₄H₁₄ClFN₂OS | 313.05 | 313.0 |
| 31 | S | S | H | Cl | F | H | H | C₁₄H₁₄ClFN₂OS | 313.05 | 313.0 |
| 32 | S | R | F | H | Cl | H | H | C₁₄H₁₄ClFN₂OS | 313.05 | 313.0 |
| 33 | S | S | F | H | Cl | H | H | C₁₄H₁₄ClFN₂OS | 313.05 | 313.0 |

```
  1 2-[(R)-((S)-Pyrrolidin-3-yl-(2,3,4-trifluorophenoxy)methyl]thiazole
  2 2-[((S)-((S)-Pyrrolidin-3-yl-(2,3,4-trifluorophenoxy)methyl]thiazole
  3 2-[(R)-((S)-Pyrrolidin-3-yl-(2,3,5,6-tetrafluorophenoxy)methyl]thiazole
  4 2-[((S)-((S)-Pyrrolidin-3-yl-(2,3,5,6-tetrafluorophenoxy)methyl]thiazole
  5 2-[((S)-Pyrrolidin-3-yl-(2,4,6-trifluorophenoxy)methyl]thiazole
  6 2-[(R)-((S)-Pyrrolidin-3-yl-(2,4,6-trifluorophenoxy)methyl]thiazole
  7 2-[((S)-((S)-Pyrrolidin-3-yl-(2,4,6-trifluorophenoxy)methyl]thiazole
  8 2-[(R)-(2,6-Dichloro-3,5-difluorophenoxy)-((S)-pyrrolidin-3-yl-methyl]thiazole
  9 2-[((S)-(2,6-Dichloro-3,5-difluorophenoxy)-((S)-pyrrolidin-3-yl-methyl]thiazole
10 2-[(R)-(2,6-Dichloro-4-fluorophenoxy)-((S)-pyrrolidin-3-yl-methyl]thiazole
11 2-[((S)-(2,6-Dichloro-4-fluorophenoxy)-((S)-pyrrolidin-3-yl-methyl]thiazole
12 2-[((S)-(2,6-Dichloro-4-methylphenoxy)-pyrrolidin-3-yl-methyl]thiazole
13 2-[(R)-(2,6-Dichloro-4-methylphenoxy)-((S)-pyrrolidin-3-yl-methyl]thiazole
14 2-[((S)-(2,6-Dichloro -4-methylphenoxy)-((S)-pyrrolidin-3-yl-methyl]thiazole
15 2-[(R)-(2-Chloro-3,6-difluorophenoxy)-((S)-pyrrolidin-3-yl-methyl]thiazole
16 2-[((S)-(2-Chloro-3,6-difluorophenoxy)-((S)-pyrrolidin-3-yl-methyl]thiazole
17 2-[((S)-(2-Chloro-6-fluoro-3-methylphenoxy)-pyrrolidin-3-yl-methyl]thiazole
18 2-[(R)-(2-Chloro-6-fluoro-3-methylphenoxy)-((S)-pyrrolidin-3-yl-methyl]thiazole
19 2-[(S)-(2-Chloro-4-fluorophenoxy)-(S)-pyrrolidin-3-ylmethyl]thiazole
20 2-[(S)-(2-Chloro-4-fluorophenoxy)-pyrrolidin-3-yl-methyl]thiazole
21 2-[(R)-(2-Chloro-4-fluorophenoxy)-(S)-pyrrolidin-3-yl-methyl]thiazole
22 2-[(R)-(4-Chloro-2-methylphenoxy)-((S)-pyrrolidin-3-yl-methyl]thiazole
23 2-[((S)-(2-Chloro-6-fluoro-3-methylphenoxy)-((S)-pyrrolidin-3-yl-methyl]thiazole
24 2-((R)-((S)-Pyrrolidin-3-yl-o-tolyloxy-methyl)thiazole
25 2-(((S)-((S)-Pyrrolidin-3-yl-o-tolyloxy-methyl)thiazole
26 2-[(R)-(3,5-Dichlorophenoxy)-((S)-pyrrolidin-3-yl-methyl]thiazole
27 2-[((S)-(3,5-Dichlorophenoxy)-((S)-pyrrolidin-3-yl-methyl]thiazole
28 2-[(R)-(2,6-Difluoro-4-methoxyphenoxy)-((S)-pyrrolidin-3-yl-methyl]thiazole
29 2-[((S)-(2,6-Difluoro-4-methoxyphenoxy)-pyrrolidin-3-yl-methyl]thiazole
30 2-[(R)-(3-Chloro-4-fluorophenoxy)-((S)-pyrrolidin-3-yl-methyl]thiazole
31 2-[((S)-(3-Chloro-4-fluorophenoxy)-((S)-pyrrolidin-3-yl-methyl]thiazole
32 2-[(R)-(4-Chloro-2-fluorophenoxy)-((S)-pyrrolidin-3-yl-methyl]thiazole
33 2-[((S)-(4-Chloro-2-fluorophenoxy)-((S)-pyrrolidin-3-yl-methyl]thiazole
```

### Example 6

Following the procedures described in the examples above, the method depicted in Scheme C, or the Mitsunobu coupling reaction, and substituting the appropriate starting materials and reagents, compounds 1 to 51, having the following formula were also prepared as TFA salts:

(II-c1)

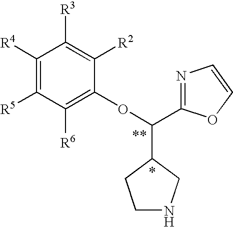

| | | | | | | | | | MS m/z: [M + H]⁺ | |
|---|---|---|---|---|---|---|---|---|---|---|
| # | * | ** | R² | R³ | R⁴ | R⁵ | R⁶ | Formula | calcd | found |
| 1 | S | R | Cl | H | Cl | Cl | H | C₁₄H₁₃Cl₃N₂O₂ | 347.00 | 347.0 |
| 2 | S | R | F | H | F | H | F | C₁₄H₁₃F₃N₂O₂ | 299.09 | 299.0 |
| 3 | S | R | Cl | H | Cl | H | H | C₁₄H₁₄Cl₂N₂O₂ | 313.04 | 313.0 |
| 4 | S | R | Cl | F | Cl | H | H | C₁₄H₁₃Cl₂FN₂O₂ | 331.03 | 331.0 |
| 5 | S | R | Cl | H | Cl | H | F | C₁₄H₁₃Cl₂FN₂O₂ | 331.03 | 331.0 |
| 6 | S | R | Cl | H | Cl | H | Me | C₁₅H₁₆Cl₂N₂O₂ | 327.06 | 327.0 |
| 7 | S | R | F | H | F | H | H | C₁₄H₁₄F₂N₂O₂ | 281.10 | 281.0 |
| 8 | S | R | Cl | H | H | H | Cl | C₁₄H₁₄Cl₂N₂O₂ | 313.04 | 313.0 |
| 9 | S | R | Cl | F | H | F | Cl | C₁₄H₁₂Cl₂F₂N₂O₂ | 349.02 | 349.0 |
| 10 | S | S | Cl | F | H | F | Cl | C₁₄H₁₂Cl₂F₂N₂O₂ | 349.02 | 349.0 |
| 11 | S | R | Cl | H | F | H | Cl | C₁₄H₁₃Cl₂FN₂O₂ | 331.03 | 331.0 |
| 12 | S | R | Cl | H | Me | H | Cl | C₁₅H₁₆Cl₂N₂O₂ | 327.06 | 327.0 |
| 13 | S | R | F | H | Cl | H | F | C₁₄H₁₃ClF₂N₂O₂ | 315.06 | 315.0 |
| 14 | S | R | Me | H | H | H | Me | C₁₆H₂₀N₂O₂ | 273.15 | 273.2 |
| 15 | S | R | Me | H | Cl | H | Me | C₁₆H₁₉ClN₂O₂ | 307.11 | 307.0 |
| 16 | S | R | Bn | H | Cl | H | H | C₂₁H₂₁ClN₂O₂ | 369.13 | 369.0 |
| 17 | S | R | CF₃ | H | H | H | H | C₁₅H₁₅F₃N₂O₂ | 313.11 | 313.0 |
| 18 | S | R | CF₃ | H | Cl | H | H | C₁₅H₁₄ClF₃N₂O₂ | 347.07 | 347.0 |
| 19 | S | R | Cl | H | H | H | H | C₁₄H₁₅ClN₂O₂ | 279.08 | 279.0 |
| 20 | S | R | Cl | F | H | H | F | C₁₄H₁₃ClF₂N₂O₂ | 315.06 | 315.0 |
| 21 | S | S | Cl | F | H | H | F | C₁₄H₁₃ClF₂N₂O₂ | 315.06 | 315.0 |
| 22 | S | R | Cl | H | F | H | H | C₁₄H₁₄ClFN₂O₂ | 297.07 | 297.0 |
| 23 | S | R | Cl | H | Me | H | H | C₁₅H₁₇ClN₂O₂ | 293.10 | 293.0 |
| 24 | S | R | Cl | H | Me | F | H | C₁₅H₁₆ClFN₂O₂ | 311.09 | 311.0 |
| 25 | S | R | Cl | H | Ph | H | H | C₂₀H₁₉ClN₂O₂ | 355.11 | 355.0 |
| 26 | S | R | Cl | H | H | H | Me | C₁₅H₁₇ClN₂O₂ | 293.10 | 293.0 |
| 27 | S | R | —COMe | Cl | Cl | H | H | C₁₆H₁₆Cl₂N₂O₃ | 355.05 | 355.0 |
| 28 | S | R | —COMe | H | F | H | F | C₁₆H₁₆F₂N₂O₃ | 323.11 | 323.0 |
| 29 | S | R | —COOMe | H | Cl | H | H | C₁₆H₁₇ClN₂O₄ | 337.09 | 337.0 |
| 30 | S | R | Et | H | F | H | H | C₁₆H₁₉FN₂O₂ | 291.14 | 291.2 |
| 31 | S | R | F | H | Cl | H | H | C₁₄H₁₄ClFN₂O₂ | 297.07 | 297.0 |
| 32 | S | R | iPro | H | H | H | H | C₁₇H₂₂N₂O₂ | 287.17 | 287.2 |
| 33 | S | R | Me | H | H | H | H | C₁₅H₁₈N₂O₂ | 259.14 | 259.2 |
| 34 | S | R | Me | H | Cl | H | H | C₁₅H₁₇ClN₂O₂ | 293.10 | 293.0 |
| 35 | S | S | Me | H | Cl | H | H | C₁₅H₁₇ClN₂O₂ | 293.10 | 293.0 |
| 36 | S | R | OBn | H | H | H | H | C₂₁H₂₂N₂O₃ | 351.16 | 351.2 |
| 37 | S | R | OCF₃ | H | H | H | H | C₁₅H₁₅F₃N₂O₃ | 329.10 | 329.0 |
| 38 | S | R | OEt | H | H | H | H | C₁₆H₂₀N₂O₃ | 289.15 | 289.0 |
| 39 | S | R | OMe | H | Cl | H | H | C₁₅H₁₇ClN₂O₃ | 309.09 | 309.0 |
| 40 | S | R | OMe | H | Cl | F | H | C₁₅H₁₆ClFN₂O₃ | 327.08 | 327.0 |
| 41 | S | R | OMe | H | H | H | F | C₁₅H₁₇FN₂O₃ | 293.12 | 293.0 |
| 42 | S | R | Ph | H | H | H | H | C₂₀H₂₀N₂O₂ | 321.15 | 321.2 |
| 43 | S | R | H | Cl | H | Cl | H | C₁₄H₁₄Cl₂N₂O₂ | 313.04 | 313.0 |
| 44 | S | S | H | Cl | H | Cl | H | C₁₄H₁₄Cl₂N₂O₂ | 313.04 | 313.0 |
| 45 | S | R | H | Cl | H | H | H | C₁₄H₁₅ClN₂O₂ | 279.08 | 279.0 |
| 46 | S | R | H | Me | H | H | H | C₁₅H₁₈N₂O₂ | 259.14 | 259.0 |
| 47 | S | R | H | H | Cl | H | H | C₁₄H₁₅ClN₂O₂ | 279.08 | 279.0 |
| 48 | S | R | H | H | Me | H | H | C₁₅H₁₈N₂O₂ | 259.14 | 259.0 |
| 49 | S | R | Cl | Cl | H | H | H | C₁₄H₁₄Cl₂N₂O₂ | 313.04 | 313.0 |
| 50 | S | R | Me | Cl | H | H | H | C₁₅H₁₇ClN₂O₂ | 293.10 | 293.0 |
| 51 | S | R | cyclohexyl | H | Cl | H | H | C₂₀H₂₅ClN₂O₂ | 361.16 | 361.2 |
| 52 | S | R | OMe | H | H | H | H | C₁₅H₁₈N₂O₃ | 275.13 | 275.0 |
| 53 | S | R | Et | H | F | H | F | C₁₆H₁₈F₂N₂O₂ | 309.13 | 309.0 |

```
  1 2-[(R)-(S)-Pyrrolidin-3-yl-(2,4,5-trichlorophenoxy)methyl]oxazole
  2 2-[(R)-(S)-Pyrrolidin-3-yl-(2,4,6-trifluorophenoxy)methyl]oxazole
  3 2-[(R)-(2,4-Dichlorophenoxy)-(S)-pyrrolidin-3-yl-methyl]oxazole
  4 2-[(R)-(2,4-Dichloro-3-fluorophenoxy)-(S)-pyrrolidin-3-yl-methyl]oxazole
  5 2-[(R)-(2,4-Dichloro-6-fluorophenoxy)-(S)-pyrrolidin-3-yl-methyl]oxazole
  6 2-[(R)-(2,4-Dichloro-6-methylphenoxy)-(S)-pyrrolidin-3-yl-methyl]oxazole
  7 2-[(R)-(2,4-Difluorophenoxy)-(S)-pyrrolidin-3-yl-methyl]oxazole
  8 2-[(R)-(2,6-Dichlorophenoxy)-(S)-pyrrolidin-3-yl-methyl]oxazole
  9 2-[(R)-(2,6-Dichloro-3,5-difluorophenoxy)-(S)-pyrrolidin-3-yl-methyl]oxazole
10 2-[(S)-(2,6-Dichloro-3,5-difluorophenoxy)-(S)-pyrrolidin-3-yl-methyl]oxazole
11 2-[(R)-(2,6-Dichloro-4-fluorophenoxy)-(S)-pyrrolidin-3-yl-methyl]oxazole
12 2-[(R)-(2,6-Dichloro-4-methylphenoxy)-(S)-pyrrolidin-3-yl-methyl]oxazole
13 2-[(R)-(4-Chloro-2,6-difluorophenoxy)-(S)-pyrrolidin-3-yl-methyl]oxazole
14 2-[(R)-(2,6-Dimethylphenoxy)-(S)-pyrrolidin-3-yl-methyl]oxazole
15 2-[(R)-(4-Chloro-2,6-dimethylphenoxy)-(S)-pyrrolidin-3-yl-methyl]oxazole
16 2-[(R)-(2-Benzyl-4-chlorophenoxy)-(S)-pyrrolidin-3-yl-methyl]oxazole
17 2-[(R)-(S)-Pyrrolidin-3-yl-(2-trifluoromethyl-phenoxy)-methyl]oxazole
18 2-[(R)-(4-Chloro-2-trifluoromethylphenoxy)-(S)-pyrrolidin-3-yl-methyl]oxazole
19 2-[(R)-(2-Chlorophenoxy)-(S)-pyrrolidin-3-yl-methyl]oxazole
20 2-[(R)-(2-Chloro-3,6-difluorophenoxy)-(S)-pyrrolidin-3-yl-methyl]oxazole
21 2-[(S)-(2-Chloro-3,6-difluorophenoxy)-(S)-pyrrolidin-3-yl-methyl]oxazole
22 2-[(R)-(2-Chloro-4-fluorophenoxy)-(S)-pyrrolidin-3-yl-methyl]oxazole
23 2-[(R)-(2-Chloro-4-methylphenoxy)-(S)-pyrrolidin-3-yl-methyl]oxazole
24 2-[(R)-(2-Chloro-5-fluoro-4-methylphenoxy)-(S)-pyrrolidin-3-yl-methyl]oxazole
25 2-[(R)-(3-Chlorobiphenyl-4-yloxy)-(S)-pyrrolidin-3-yl-methyl]oxazole
26 2-[(R)-(2-Chloro-6-methylphenoxy)-(S)-pyrrolidin-3-yl-methyl]oxazole
27 1-[2,3-Dichloro-6-((R)-oxazol-2-yl-(S)-pyrrolidin-3-yl-methoxy)phenyl]ethanone
28 1-[3,5-Difluoro-2-((R)-oxazol-2-yl-(S)-pyrrolidin-3-yl-methoxy)phenyl]ethanone
29 5-Chloro-2-((R)-oxazol-2-yl-(S)-pyrrolidin-3-yl-methoxy)benzoic acid methyl ester
30 2-[(R)-(2-Ethyl-4-fluorophenoxy)-(S)-pyrrolidin-3-yl-methyl]oxazole
31 2-[(R)-(4-Chloro-2-fluorophenoxy)-(S)-pyrrolidin-3-yl-methyl]oxazole
32 2-[(R)-(2-Isopropylphenoxy)-(S)-pyrrolidin-3-yl-methyl]oxazole
33 2-((R)-(S)-Pyrrolidin-3-yl-o-tolyloxymethyl)oxazole
34 2-[(R)-(4-Chloro-2-methylphenoxy)-(S)-pyrrolidin-3-yl-methyl]oxazole
35 2-[(S)-(4-Chloro-2-methylphenoxy)-(S)-pyrrolidin-3-yl-methyl]oxazole
36 2-[(R)-(2-Benzyloxyphenoxy)-(S)-pyrrolidin-3-yl-methyl]oxazole
37 2-[(R)-(S)-Pyrrolidin-3-yl-(2-trifluoromethoxyphenoxy)methyl]oxazole
38 2-[(R)-(2-Ethoxyphenoxy)-(S)-pyrrolidin-3-yl-methyl]oxazole
39 2-[(R)-(4-Chloro-2-methoxyphenoxy)-(S)-pyrrolidin-3-yl-methyl]oxazole
40 2-[(R)-(4-Chloro-5-fluoro-2-methoxyphenoxy)-(S)-pyrrolidin-3-yl-methyl]oxazole
41 2-[(R)-(2-Fluoro-6-methoxyphenoxy)-(S)-pyrrolidin-3-yl-methyl]oxazole
42 2-[(R)-(Biphenyl-2-yloxy)-(S)-pyrrolidin-3-yl-methyl]oxazole
43 2-[(R)-(3,5-Dichlorophenoxy)-(S)-pyrrolidin-3-yl-methyl]oxazole
44 2-[(S)-(3,5-Dichlorophenoxy)-(S)-pyrrolidin-3-yl-methyl]oxazole
45 2-[(R)-(3-Chlorophenoxy)-(S)-pyrrolidin-3-yl-methyl]oxazole
46 2-((R)-(S)-Pyrrolidin-3-yl-m-tolyloxymethyl)oxazole
47 2-[(R)-(4-Chlorophenoxy)-(S)-pyrrolidin-3-yl-methyl]oxazole
48 2-((R)-(S)-Pyrrolidin-3-yl-p-tolyloxymethyl)oxazole
49 2-[(R)-(2,3-Dichlorophenoxy)-(S)-pyrrolidin-3-yl-methyl]oxazole
50 2-[(R)-(3-Chloro-2-methyl-phenoxy)-(S)-pyrrolidin-3-yl-methyl]oxazole
51 2-[(R)-(4-Chloro-2-cyclohexylphenoxy)-(S)-pyrrolidin-3-yl-methyl]oxazole
52 2-[(R)-(2-Methoxyphenoxy)-(S)-pyrrolidin-3-yl-methyl]oxazole
53 2-[(R)-(2-Ethyl-4,6-difluorophenoxy)-(S)-pyrrolidin-3-yl-methyl]oxazole
```

### Example 7

Following the procedures described in the examples above, the method depicted in Scheme C, or the Mitsunobu coupling reaction, and substituting the appropriate starting materials and reagents, compounds 1 to 3, having the following formula were also prepared as TFA salts:

(II-c2)

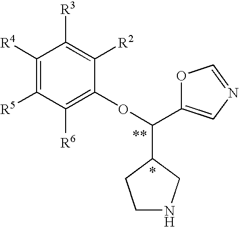

| | | | | | | | | | MS m/z: | |
|---|---|---|---|---|---|---|---|---|---|---|
| | | | | | | | | | [M + H]⁺ | |
| # | * | ** | R² | R³ | R⁴ | R⁵ | R⁶ | Formula | calcd | found |
| 1 | S | R | H | Cl | H | Cl | H | C₁₄H₁₄Cl₂N₂O₂ | 313.04 | 313.0 |
| 2 | S | R | Cl | F | H | H | F | C₁₄H₁₃ClF₂N₂O₂ | 315.06 | 315.0 |
| 3 | S | R | Me | H | Cl | H | H | C₁₅H₁₇ClN₂O₂ | 293.10 | 293.0 |

1 5-[(R)-(3,5-Dichlorophenoxy)-(S)-pyrrolidin-3-ylmethyl]oxazole
2 5-[(R)-(2-Chloro-3,6-difluorophenoxy)-(S)-pyrrolidin-3-ylmethyl]oxazole
3 5-[(R)-(4-Chloro-2-methylphenoxy)-(S)-pyrrolidin-3-ylmethyl]oxazole

### Example 8

Following the procedures described in the examples above, the method depicted in Scheme C, or the Mitsunobu coupling reaction, and substituting the appropriate starting materials and reagents, compounds 1 to 4, having the following formula were also prepared as TFA salts:

(II-d1)

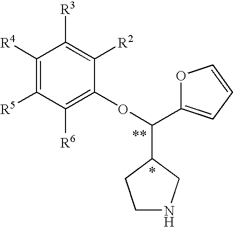

| | | | | | | | | | MS m/z: [M + H]⁺ | |
|---|---|---|---|---|---|---|---|---|---|---|
| # | * | ** | R² | R³ | R⁴ | R⁵ | R⁶ | Formula | calcd | found |
| 1 | S | R | Cl | F | H | H | F | C₁₅H₁₄ClF₂NO₂ | 314.07 | 314.0 |
| 2 | S | R | Me | H | Cl | H | H | C₁₆H₁₈ClNO₂ | 292.10 | 292.0 |
| 3 | S | R | H | Cl | H | Cl | H | C₁₅H₁₅Cl₂NO₂ | 312.05 | 312.0 |
| 4 | S | S | H | Cl | H | Cl | H | C₁₅H₁₅Cl₂NO₂ | 312.05 | 312.0 |

1 (S)-3-[(R)-(2-Chloro-3,6-difluorophenoxy)furan-2-yl-methyl]pyrrolidine
2 (S)-3-[(R)-(4-Chloro-2-methylphenoxy)furan-2-yl-methyl]pyrrolidine
3 (S)-3-[(R)-(3,5-Dichlorophenoxy)furan-2-yl-methyl]pyrrolidine
4 (S)-3-[(S)-(3,5-Dichlorophenoxy)furan-2-yl-methyl]pyrrolidine

### Example 9

Following the procedures described in the examples above, the method depicted in Scheme C, or the Mitsunobu coupling reaction, and substituting the appropriate starting materials and reagents, compound 1, having the following formula was also prepared as a TFA salt:

(II-d2)

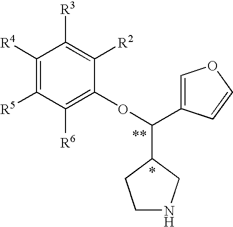

| | | | | | | | | | MS m/z: [M + H]⁺ | |
|---|---|---|---|---|---|---|---|---|---|---|
| # | * | ** | R² | R³ | R⁴ | R⁵ | R⁶ | Formula | calcd | found |
| 1 | S | R | H | Cl | H | Cl | H | C₁₅H₁₅Cl₂NO₂ | 312.05 | 312.0 |

1 (S)-3-[(R)-(3,5-Dichlorophenoxy)-furan-3-yl-methyl]pyrrolidione

### Example 10

Following the procedures described in the examples above, the method depicted in Scheme C, or the Mitsunobu coupling reaction, and substituting the appropriate starting materials and reagents, compounds 1 to 10, having the following formula were also prepared as TFA salts:

(II-e)

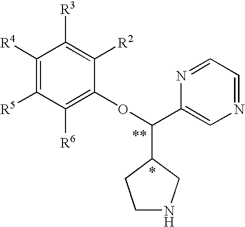

| | | | | | | | | | MS m/z: [M + H]⁺ | |
|---|---|---|---|---|---|---|---|---|---|---|
| # | * | ** | R² | R³ | R⁴ | R⁵ | R⁶ | Formula | calcd | found |
| 1 | S | R | Cl | F | H | F | Cl | C₁₅H₁₃Cl₂F₂N₃O | 360.04 | 360.0 |
| 2 | S | S | Cl | F | H | F | Cl | C₁₅H₁₃Cl₂F₂N₃O | 360.04 | 360.0 |
| 3 | S | R | Cl | F | H | H | F | C₁₅H₁₄ClF₂N₃O | 326.08 | 326.0 |
| 4 | S | S | Cl | F | H | H | F | C₁₅H₁₄ClF₂N₃O | 326.08 | 326.0 |
| 5 | S | R | Me | H | Cl | H | H | C₁₆H₁₈ClN₃O | 304.11 | 304.0 |
| 6 | S | S | Me | H | Cl | H | H | C₁₆H₁₈ClN₃O | 304.11 | 304.0 |
| 7 | S | R | OMe | H | H | H | H | C₁₆H₁₉N₃O₂ | 286.15 | 286.2 |
| 8 | S | S | OMe | H | H | H | H | C₁₆H₁₉N₃O₂ | 286.15 | 286.2 |
| 9 | S | R | H | Cl | H | Cl | H | C₁₅H₁₅Cl₂N₃O | 324.06 | 324.0 |
| 10 | S | S | H | Cl | H | Cl | H | C₁₅H₁₅Cl₂N₃O | 324.06 | 324.0 |

```
  1 2-[(R)-(2,6-Dichloro-3,5-difluorophenoxy)-(S)-pyrrolidin-3-yl-methyl]pyrazine
  2 2-[(S)-(2,6-Dichloro-3,5-difluorophenoxy)-(S)-pyrrolidin-3-yl-methyl]pyrazine
  3 2-[(R)-(2-Chloro-3,6-difluorophenoxy)-(S)-pyrrolidin-3-yl-methyl]pyrazine
  4 2-[(S)-(2-Chloro-3,6-difluorophenoxy)-(S)-pyrrolidin-3-yl-methyl]pyrazine
  5 2-[(R)-(4-Chloro-2-methylphenoxy)-(S)-pyrrolidin-3-yl-methyl]pyrazine
  6 2-[(S)-(4-Chloro-2-methylphenoxy)-(S)-pyrrolidin-3-yl-methyl]pyrazine
  7 2-[(R)-(2-Methoxyphenoxy)-(S)-pyrrolidin-3-yl-methyl]pyrazine
  8 2-[(S)-(2-Methoxyphenoxy)-(S)-pyrrolidin-3-yl-methyl]pyrazine
  9 2-[(R)-(3,5-Dichlorophenoxy)-(S)-pyrrolidin-3-yl-methyl]pyrazine
10 2-[(S)-(3,5-Dichlorophenoxy)-(S)-pyrrolidin-3-yl-methyl]pyrazine
```

### Example 11

Following the procedures described in the examples above, the method depicted in Scheme C, or the Mitsunobu coupling reaction, and substituting the appropriate starting materials and reagents, compounds 1 to 5, having the following formula were also prepared as TFA salts:

(II-f1)

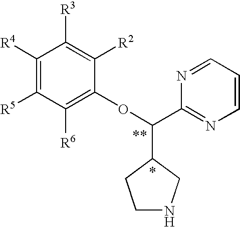

| | | | | | | | | | MS m/z: [M + H]⁺ | |
|---|---|---|---|---|---|---|---|---|---|---|
| # | * | ** | R² | R³ | R⁴ | R⁵ | R⁶ | Formula | calcd | found |
| 1 | S | R | Me | H | Cl | H | H | C₁₆H₁₈ClN₃O | 304.11 | 304.0 |
| 2 | S | R | Cl | F | H | H | F | C₁₅H₁₄ClF₂N₃O | 326.08 | 326.0 |
| 3 | S | R | Cl | F | H | F | Cl | C₁₅H₁₃Cl₂F₂N₃O | 360.04 | 360.0 |
| 4 | S | R | OMe | H | H | H | H | C₁₆H₁₉N₃O₂ | 286.15 | 286.2 |
| 5 | S | R | H | Cl | H | Cl | H | C₁₅H₁₅Cl₂N₃O | 324.06 | 324.0 |

1 2-[(R)-(4-Chloro-2-methylphenoxy)-(S)-pyrrolidin-3-yl-methyl]pyrimidine
2 2-[(R)-(2-Chloro-3,6-difluorophenoxy)-(S)-pyrrolidin-3-yl-methyl]pyrimidine
3 2-[(R)-(2,6-Dichloro-3,5-difluorophenoxy)-(S)-pyrrolidin-3-yl-methyl]pyrimidine
4 2-[(R)-(2-Methoxyphenoxy)-(S)-pyrrolidin-3-yl-methyl]pyrimidine
5 2-[(R)-(3,5-Dichlorophenoxy)-(S)-pyrrolidin-3-yl-methyl]pyrimidine

### Example 12

Following the procedures described in the examples above, the method depicted in Scheme C, or the Mitsunobu coupling reaction, and substituting the appropriate starting materials and reagents, compounds 1 to 5, having the following formula were also prepared as TFA salts:

(II-f2)

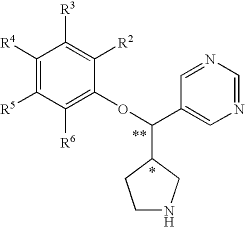

| | | | | | | | | | MS m/z: | |
|---|---|---|---|---|---|---|---|---|---|---|
| | | | | | | | | | [M + H]⁺ | |
| # | * | ** | R² | R³ | R⁴ | R⁵ | R⁶ | Formula | calcd | found |
| 1 | S | R | Cl | F | H | H | F | C₁₅H₁₄ClF₂N₃O | 326.08 | 326.0 |
| 2 | S | S | Cl | F | H | H | F | C₁₅H₁₄ClF₂N₃O | 326.08 | 326.0 |
| 3 | S | R | Me | H | Cl | H | H | C₁₆H₁₈ClN₃O | 304.11 | 304.0 |
| 4 | S | R | OMe | H | H | H | H | C₁₆H₁₉N₃O₂ | 286.15 | 286.2 |
| 5 | S | R | H | H | H | Cl | H | C₁₅H₁₅Cl₂N₃O | 324.06 | 324.0 |

1 5-[(R)-(2-Chloro-3,6-difluorophenoxy)-(S)-pyrrolidin-3-ylmethyl]
pyrimidine
2 5-[(S)-(2-Chloro-3,6-difluorophenoxy)-(S)-pyrrolidin-3-ylmethyl]
pyrimidine
3 5-[(R)-(4-Chloro-2-methylphenoxy)-(S)-pyrrolidin-3-ylmethyl]
pyrimidine
4 5-[(R)-(2-Methoxyphenoxy)-(S)-pyrrolidin-3-ylmethyl]pyrimidine
5 5-[(R)-(3,5-Dichlorophenoxy)-(S)-pyrrolidin-3-ylmethyl]
pyrimidine

### Preparation 3

### (S)-3((R)-Hydroxyphenylmethyl)pyrrolidine-1-carboxylic Acid t-Butyl Ester and (S)-3-((S)-Hydroxyphenylmethyl)pyrrolidine-1-carboxylic Acid t-Butyl Ester

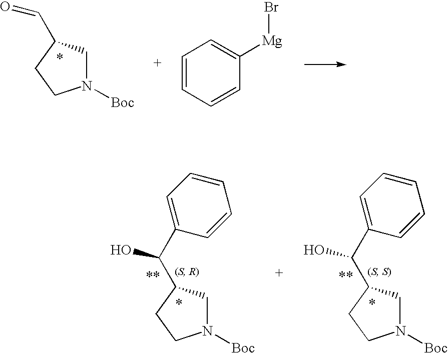

(S)-3-Formylpyrrolidine-1-carboxylic acid t-butyl ester (5.8 g, 29.1 mmol) in THF (140 mL, 1.7 mol) was placed in a flask under nitrogen, and the solution was cooled to −78° C. 1.0 M of phenyl magnesium bromide in THF (43.7 mL, 43.7 mmol) was added dropwise over 20 minutes. The solution was allowed to warm to room temperature overnight, then 250 mL saturated NH₄Cl was added dropwise to quench the reaction. The resulting mixture was extracted with EtOAc (3×150 mL), and the combined organic layers were washed with saturated aqueous NaHCO₃ (1×100 mL) and saturated aqueous NaCl (1×100 mL), then dried over anhydrous Na₂SO₄, filtered, and concentrated in vacuo. The product was combined with a separate lot of material, purified and separated by preparative HPLC to yield 3.2 g of the (S,R) and 3.1 g of the (S,S) title compounds.

### Example 13

### 5-Chloro-3-methyl-2-((R)-phenyl-(S)-pyrrolidin-3-ylmethoxy)pyridine

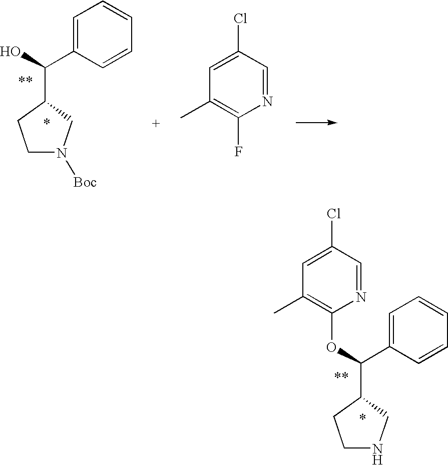

To a vial under an atmosphere of nitrogen was added (S)-3-((R)-hydroxyphenylmethyl)pyrrolidine-1-carboxylic acid t-butyl ester (35 mg, 130 μmol, 1 equiv) and anhydrous DMF (1.0 ml, 13 mmol). Sodium hydride (3.6 mg, 150 μmol, 1.2 equiv), which had been washed (3×5 ml hexanes) and dried under vacuum, was then added. This cloudy mixture was stirred at room temperature for 15 minutes then it was decanted into a vial containing 5-chloro-2-fluoro-3-picoline (55 mg, 380 μmol, 3.0 equiv). This mixture was then capped and heated at 70° C. for three hours. After cooling the mixture to room temperature, the solvent was removed by rotary evaporation. The crude material was dissolved in a solution of HCl in EtOH (1.2M, 610 μL, 5.8 equiv) and the resultant solution was stirred overnight at room temperature. The solution was then concentrated by rotary evaporation to provide an oil. This residue was redissolved in a solution of 50% AcOH in water (5 ml) and purified by preparative HPLC on a 1 inch BDS-packed column (50 minute gradient; 10 to 70% ACN in water containing 0.05% TFA). Lyophilization of the fractions provided the TFA salt of the title compound in 93% purity (28 mg, 52% yield). MS m/z: [M+H]⁺ calcd for C₁₇H₁₉ClN₂O, 303.12; found: 303.3.

### Example 14

Following the procedures described in the examples above or in the Schemes, and substituting the appropriate starting materials and reagents, compounds 1 to 42, having the following formula were also prepared as TFA salts:

(III-a1)

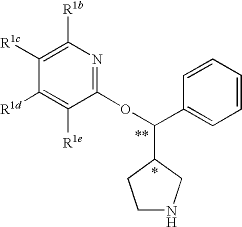

| | | | | | | | | MS m/z: [M + H]⁺ | |
|---|---|---|---|---|---|---|---|---|---|
| # | * | ** | R^{1b} | R^{1c} | R^{1d} | R^{1e} | Formula | calcd | found |
| 1 | S | R | F | F | H | F | C₁₆H₁₅F₃N₂O | 309.11 | 309.0 |
| 2 | S | R | F | F | —CN | F | C₁₇H₁₄F₃N₃O | 334.11 | 334.0 |
| 3 | S | R | F | F | —N(CH₃)₂ | F | C₁₈H₂₀F₃N₃O | 352.16 | 352.2 |
| 4 | S | R | F | F | Me | F | C₁₇H₁₇F₃N₂O | 323.13 | 323.0 |
| 5 | S | R | F | F | H | H | C₁₆H₁₆F₂N₂O | 291.12 | 291.0 |
| 6 | S | R | Cl | F | Cl | F | C₁₆H₁₄Cl₂F₂N₂O | 359.05 | 359.0 |
| 7 | S | R | F | H | F | H | C₁₆H₁₆F₂N₂O | 291.12 | 291.0 |
| 8 | S | R | F | Cl | F | Cl | C₁₆H₁₄Cl₂F₂NO | 359.05 | 359.0 |
| 9 | S | R | Cl | H | H | H | C₁₆H₁₇ClN₂O | 289.10 | 289.0 |
| 10 | S | R | F | H | H | H | C₁₆H₁₇FN₂O | 273.13 | 273.2 |
| 11 | S | R | Me | H | H | H | C₁₇H₂₀N₂O | 269.16 | 269.2 |
| 12 | S | R | Me | Br | H | Br | C₁₇H₁₈Br₂N₂O | 424.98 | 425.0 |
| 13 | S | R | H | Br | H | Br | C₁₆H₁₆Br₂N₂O | 410.96 | 410.8 |
| 14 | S | R | H | Cl | H | Cl | C₁₆H₁₆Cl₂N₂O | 323.06 | 323.0 |
| 15 | R | S | H | Cl | H | Cl | C₁₆H₁₆Cl₂N₂O | 323.06 | 323.0 |
| 16 | R | S | H | F | H | F | C₁₆H₁₆F₂N₂O | 291.12 | 291.2 |
| 17 | S | R | H | F | H | F | C₁₆H₁₆F₂N₂O | 291.12 | 291.0 |
| 18 | S | R | H | F | —CF₃ | F | C₁₇H₁₅F₅N₂O | 359.11 | 359.0 |
| 19 | R | S | H | Br | H | H | C₁₆H₁₇BrN₂O | 333.05 | 333.0 |
| 20 | S | R | H | Br | H | H | C₁₆H₁₇BrN₂O | 333.05 | 333.0 |
| 21 | S | R | H | Br | H | Me | C₁₇H₁₉BrN₂O | 347.07 | 347.0 |
| 22 | S | R | H | Br | H | OMe | C₁₇H₁₉BrN₂O₂ | 363.06 | 363.0 |
| 23 | S | R | H | —CF₃ | H | H | C₁₇H₁₇F₃N₂O | 323.13 | 323.2 |
| 24 | R | S | H | —CF₃ | H | H | C₁₇H₁₇F₃N₂O | 323.13 | 323.2 |
| 25 | S | R | H | —CF₃ | H | Cl | C₁₇H₁₆ClF₃N₂O | 357.09 | 357.0 |
| 26 | S | R | H | Cl | H | H | C₁₆H₁₇ClN₂O | 289.10 | 289.0 |
| 27 | R | S | H | Cl | H | H | C₁₆H₁₇ClN₂O | 289.10 | 289.0 |
| 28 | S | R | H | Cl | H | F | C₁₆H₁₆ClFN₂O | 307.09 | 307.0 |
| 29 | S | R | H | F | H | H | C₁₆H₁₇FN₂O | 273.13 | 273.2 |
| 30 | S | R | H | Me | H | H | C₁₇H₂₀N₂O | 269.16 | 269.2 |
| 31 | S | R | H | Me | H | Br | C₁₇H₁₉BrN₂O | 347.07 | 347.0 |
| 32 | S | R | H | H | Me | H | C₁₇H₂₀N₂O | 269.16 | 269.2 |
| 33 | R | S | H | H | H | —CF₃ | C₁₇H₁₇F₃N₂O | 323.13 | 323.2 |
| 34 | S | R | H | H | H | —CF₃ | C₁₇H₁₇F₃N₂O | 323.13 | 323.2 |
| 35 | R | S | H | H | H | F | C₁₆H₁₇FN₂O | 273.13 | 273.2 |
| 36 | S | R | H | H | H | F | C₁₆H₁₇FN₂O | 273.13 | 273.2 |
| 37 | S | R | H | H | H | Me | C₁₇H₂₀N₂O | 269.16 | 269.2 |
| 38 | R | S | H | H | H | Me | C₁₇H₂₀N₂O | 269.16 | 269.2 |
| 39 | S | R | H | H | H | OMe | C₁₇H₂₀N₂O₂ | 285.15 | 285.2 |
| 40 | RS/SR | | H | H | H | H | C₁₆H₁₈N₂O | 255.14 | 255.2 |
| 41 | S | R | H | H | H | H | C₁₆H₁₈N₂O | 255.14 | 255.2 |
| 42 | S | R | F | F | F | F | C₁₆H₁₄F₄N₂O | 327.10 | 327.0 |

```
  1 2,3,5-Trifluoro-6-((R)-phenyl-(S)-pyrrolidin-3-ylmethoxy)pyridine
  2 2,3,5-Trifluoro-6-((R)-phenyl-(S)-pyrrolidin-3-ylmethoxy)isonicotinonitrile
  3 Dimethyl-[2,3,5-trifluoro-6-((R)-phenyl-(S)-pyrrolidin-3-ylmethoxy)pyridin-4-yl]amine
  4 2,3,5-Trifluoro-4-methyl-6-((R)-phenyl-(S)-pyrrolidin-3-ylmethoxy)pyridine
  5 2,3-Difluoro-6-((R)-phenyl-(S)-pyrrolidin-3-ylmethoxy)pyridine
  6 2,4-Dichloro-3,5-difluoro-6-((R)-phenyl-(S)-pyrrolidin-3-ylmethoxy)pyridine
  7 2,4-Difluoro-6-((R)-phenyl-(S)-pyrrolidin-3-ylmethoxy)pyridine
  8 3,5-Dichloro-2,4-difluoro-6-((R)-phenyl-(S)-pyrrolidin-3-ylmethoxy)pyridine
  9 2-Chloro-6-((R)-phenyl-(S)-pyrrolidin-3-ylmethoxy)pyridine
10 2-Fluoro-6-((R)-phenyl-(S)-pyrrolidin-3-ylmethoxy)pyridine
11 2-Methyl-6-((R)-phenyl-(S)-pyrrolidin-3-ylmethoxy)pyridine
12 3,5-Dibromo-2-methyl-6-((R)-phenyl-(S)-pyrrolidin-3-ylmethoxy)pyridine
13 3,5-Dibromo-2-((R)-phenyl-(S)-pyrrolidin-3-ylmethoxy)pyridine
14 3,5-Dichloro-2-((R)-phenyl-(S)-pyrrolidin-3-ylmethoxy)pyridine
15 3,5-Dichloro-2-((S)-phenyl-(R)-pyrrolidin-3-ylmethoxy)pyridine
16 3,5-Difluoro-2-((S)-phenyl-(R)-pyrrolidin-3-ylmethoxy)pyridine
17 3,5-Difluoro-2-((R)-phenyl-(S)-pyrrolidin-3-ylmethoxy)pyridine
18 3,5-Difluoro-2-((R)-phenyl-(S)-pyrrolidin-3-ylmethoxy)-4-trifluoromethylpyridine
19 5-Bromo-2-((S)-phenyl-(R)-pyrrolidin-3-ylmethoxy)pyridine
20 5-Bromo-2-((R)-phenyl-(S)-pyrrolidin-3-ylmethoxy)pyridine
21 5-Bromo-3-methyl-2-((R)-phenyl-(S)-pyrrolidin-3-ylmethoxy)pyridine
22 5-Bromo-3-methoxy-2-((R)-phenyl-(S)-pyrrolidin-3-ylmethoxy)pyridine
23 2-((R)-Phenyl-(S)-pyrrolidin-3-ylmethoxy)-5-trifluoromethylpyridine
24 2-((S)-Phenyl-(R)-pyrrolidin-3-ylmethoxy)-5-trifluoromethylpyridine
25 3-Chloro-2-((R)-phenyl-(S)-pyrrolidin-3-ylmethoxy)-5-trifluoromethylpyridine
26 5-Chloro-2-((R)-phenyl-(S)-pyrrolidin-3-ylmethoxy)pyridine
27 5-Chloro-2-((S)-phenyl-(R)-pyrrolidin-3-ylmethoxy)pyridine
28 5-Chloro-3-fluoro-2-((R)-phenyl-(S)-pyrrolidin-3-ylmethoxy)pyridine
29 5-Fluoro-2-((R)-phenyl-(S)-pyrrolidin-3-ylmethoxy)pyridine
30 5-Methyl-2-((R)-phenyl-(S)-pyrrolidin-3-ylmethoxy)pyridine
31 3-Bromo-5-methyl-2-((R)-phenyl-(S)-pyrrolidin-3-ylmethoxy)pyridine
32 4-Methyl-2-((R)-phenyl-(S)-pyrrolidin-3-ylmethoxy)pyridine
33 2-((S)-Phenyl-(R)-pyrrolidin-3-ylmethoxy)-3-trifluoromethylpyridine
34 2-((R)-Phenyl-(S)-pyrrolidin-3-ylmethoxy)-3-trifluoromethylpyridine
35 3-Fluoro-2-((S)-phenyl-(R)-pyrrolidin-3-ylmethoxy)pyridine
36 3-Fluoro-2-((R)-phenyl-(S)-pyrrolidin-3-ylmethoxy)pyridine
37 3-Methyl-2-((R)-phenyl-(S)-pyrrolidin-3-ylmethoxy)pyridine
38 3-Methyl-2-((S)-phenyl-(R)-pyrrolidin-3-ylmethoxy)pyridine
39 3-Methoxy-2-((R)-phenyl-(S)-pyrrolidin-3-ylmethoxy)pyridine
40 2-(Phenyl-pyrrolidin-3-yl-methoxy)pyridine
41 2-((R)-Phenyl-(S)-pyrrolidin-3-yl-methoxy)pyridine
42 2,3,4,5-Tetrafluoro-6-((R)-phenyl-(S)-pyrrolidin-3-yl-methoxy)pyridine
```

### Example 15

Following the procedures described in the examples above or in the Schemes, and substituting the appropriate starting materials and reagents, compounds 1 to 32, having the following formula were also prepared as TFA salts:

(III-a2)

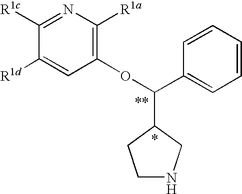

| | | | | | | | MS m/z: [M + H]⁺ | |
|---|---|---|---|---|---|---|---|---|
| # | * | ** | R^{1a} | R^{1c} | R^{1d} | Formula | calcd | found |
| 1 | RS/SR | | Cl | Cl | H | C₁₆H₁₆Cl₂N₂O | 323.06 | 323.0 |
| 2 | R | S | Cl | Cl | H | C₁₆H₁₆Cl₂N₂O | 323.06 | 323.0 |
| 3 | S | R | Cl | Cl | H | C₁₆H₁₆Cl₂N₂O | 323.06 | 323.0 |
| 4 | RS/SR | | Cl | H | H | C₁₆H₁₇ClN₂O | 289.10 | 289.0 |
| 5 | R | S | Cl | H | H | C₁₆H₁₇ClN₂O | 289.10 | 289.0 |
| 6 | S | R | Cl | H | H | C₁₆H₁₇ClN₂O | 289.10 | 289.0 |
| 7 | R | S | F | H | H | C₁₆H₁₇FN₂O | 273.13 | 273.2 |
| 8 | S | R | F | H | H | C₁₆H₁₇FN₂O | 273.13 | 273.2 |
| 9 | S | R | Me | H | H | C₁₇H₂₀N₂O | 269.16 | 269.2 |
| 10 | R | S | —O—CH₂—CF₃ | H | H | C₁₈H₁₉F₃N₂O₂ | 353.14 | 353.2 |
| 11 | S | R | —O—CH₂—CF₃ | H | H | C₁₇H₁₉F₃N₂O₂ | 353.14 | 353.2 |
| 12 | R | S | —O-cyclopentyl | H | H | C₂₁H₂₆N₂O₂ | 339.20 | 339.2 |
| 13 | S | R | —O-cyclopentyl | H | H | C₂₁H₂₆N₂O₂ | 339.20 | 339.2 |
| 14 | R | S | —O—CH—(CH₃)₂ | H | H | C₁₉H₂₄N₂O₂ | 313.18 | 313.2 |
| 15 | S | R | —O—CH—(CH₃)₂ | H | H | C₁₉H₂₄N₂O₂ | 313.18 | 313.2 |
| 16 | RS/SR | | OMe | H | H | C₁₇H₂₀N₂O₂ | 285.15 | 285.2 |
| 17 | S | R | OMe | H | H | C₁₇H₂₀N₂O₂ | 285.15 | 285.2 |
| 18 | R | S | OMe | H | H | C₁₇H₂₀N₂O₂ | 285.15 | 285.4 |
| 19 | S | R | —O—(CH₂)₂—CH₃ | H | H | C₁₉H₂₄N₂O₂ | 313.18 | 313.2 |
| 20 | S | R | —O—CH₂—CH₃ | H | H | C₁₉H₂₂N₂O₂ | 299.17 | 299.2 |
| 21 | S | R | H | F | F | C₁₆H₁₆F₂N₂O | 291.12 | 291.2 |
| 22 | RS/SR | | H | Cl | H | C₁₆H₁₇ClN₂O | 289.10 | 289.0 |
| 23 | R | S | H | Cl | H | C₁₆H₁₇ClN₂O | 289.10 | 289.0 |
| 24 | S | R | H | Cl | H | C₁₆H₁₇ClN₂O | 289.10 | 289.0 |
| 25 | RS/SR | | H | F | H | C₁₆H₁₇FN₂O | 273.13 | 273.0 |
| 26 | R | S | H | F | H | C₁₆H₁₇FN₂O | 273.13 | 273.2 |
| 27 | S | R | H | F | H | C₁₆H₁₇FN₂O | 273.13 | 273.2 |
| 28 | S | R | H | Me | H | C₁₇H₂₀N₂O | 269.16 | 269.2 |
| 29 | RS/SR | | H | OMe | H | C₁₇H₂₀N₂O₂ | 285.15 | 285.2 |
| 30 | R | S | H | OMe | H | C₁₇H₂₀N₂O₂ | 285.15 | 285.2 |
| 31 | S | R | H | OMe | H | C₁₇H₂₀N₂O₂ | 285.15 | 285.2 |

| | | | | | | | |
|---|---|---|---|---|---|---|---|
| 32 | RS/SR | H | H | H | C₁₆H₁₈N₂O | 255.14 | 255.2 |

```
  1 2,6-Dichloro-3-((R)-phenyl-pyrrolidin-3-ylmethoxy)pyridine
  2 2,6-Dichloro-3-((S)-phenyl-(R)-pyrrolidin-3-ylmethoxy)pyridine
  3 2,6-Dichloro-3-((R)-phenyl-(S)-pyrrolidin-3-ylmethoxy)pyridine
  4 2-Chloro-3-((R)-phenyl-pyrrolidin-3-ylmethoxy)pyridine
  5 2-Chloro-3-((S)-phenyl-(R)-pyrrolidin-3-ylmethoxy)pyridine
  6 2-Chloro-3-((R)-phenyl-(S)-pyrrolidin-3-ylmethoxy)pyridine
  7 2-Fluoro-3-((S)-phenyl-(R)-pyrrolidin-3-ylmethoxy)pyridine
  8 2-Fluoro-3-((R)-phenyl-(S)-pyrrolidin-3-ylmethoxy)pyridine
  9 2-Methyl-3-((R)-phenyl-(S)-pyrrolidin-3-ylmethoxy)pyridine
10 3-((S)-Phenyl-(R)-pyrrolidin-3-ylmethoxy)-2-(2,2,2-trifluoroethoxy)pyridine
11 3-((R)-Phenyl-(S)-pyrrolidin-3-ylmethoxy)-2-(2,2,2-trifluoroethoxy)pyridine
12 2-Cyclopentyloxy-3-((S)-phenyl-(R)-pyrrolidin-3-ylmethoxy)pyridine
13 2-Cyclopentyloxy-3-((R)-phenyl-(S)-pyrrolidin-3-ylmethoxy)pyridine
14 2-Isopropoxy-3-((S)-phenyl-(R)-pyrrolidin-3-ylmethoxy)pyridine
15 2-Isopropoxy-3-((R)-phenyl-(S)-pyrrolidin-3-ylmethoxy)pyridine
16 2-Methoxy-3-((R)-phenyl-pyrrolidin-3-ylmethoxy)pyridine
17 2-Methoxy-3-((R)-phenyl-(S)-pyrrolidin-3-ylmethoxy)pyridine
18 2-Methoxy-3-((S)-phenyl-(R)-pyrrolidin-3-ylmethoxy)pyridine
19 3-((R)-Phenyl-(S)-pyrrolidin-3-ylmethoxy)-2-propoxypyridine
20 2-Ethoxy-3-((R)-phenyl-(S)-pyrrolidin-3-ylmethoxy)pyridine
21 2,3-Difluoro-5-((R)-phenyl-(S)-pyrrolidin-3-ylmethoxy)pyridine
22 2-Chloro-5-((R)-phenyl-pyrrolidin-3-ylmethoxy)pyridine
23 2-Chloro-5-((S)-phenyl-(R)-pyrrolidin-3-ylmethoxy)pyridine
24 2-Chloro-5-((R)-phenyl-(S)-pyrrolidin-3-ylmethoxy)pyridine
25 2-Fluoro-5-((R)-phenyl-pyrrolidin-3-ylmethoxy)pyridine
26 2-Fluoro-5-((S)-phenyl-(R)-pyrrolidin-3-ylmethoxy)pyridine
27 2-Fluoro-5-((R)-phenyl-(S)-pyrrolidin-3-ylmethoxy)pyridine
28 2-Methyl-5-((R)-phenyl-(S)-pyrrolidin-3-ylmethoxy)pyridine
29 2-Methoxy-5-((R)-phenyl-pyrrolidin-3-ylmethoxy)pyridine
30 2-Methoxy-5-((S)-phenyl-(R)-pyrrolidin-3-ylmethoxy)pyridine
31 2-Methoxy-5-((R)-phenyl-(S)-pyrrolidin-3-ylmethoxy)pyridine
32 3-(Phenylpyrrolidin-3-yl-methoxy)pyridine
```

### Example 16

Following the procedures described in the examples above or in the Schemes, and substituting the appropriate starting materials and reagents, compounds 1 to 6, having the following formula were also prepared as TFA salts:

(III-a3)

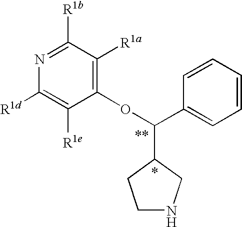

| | | | | | | | | MS m/z: [M + H]⁺ | |
|---|---|---|---|---|---|---|---|---|---|
| # | * | ** | R^{1a} | R^{1b} | R^{1d} | R^{1e} | Formula | calcd | found |
| 1 | S | R | Me | Cl | H | H | C₁₇H₁₉ClN₂O | 303.12 | 303.0 |
| 2 | S | R | H | Cl | Cl | H | C₁₆H₁₆Cl₂N₂O | 323.06 | 323.0 |
| 3 | RS/SR | | H | Cl | H | H | C₁₆H₁₇ClN₂O | 289.10 | 289.0 |
| 4 | S | R | H | CN | H | H | C₁₇H₁₇N₃O | 280.14 | 280.2 |
| 5 | R | S | H | F | H | H | C₁₆H₁₇FN₂O | 273.13 | 273.0 |
| 6 | S | R | H | F | H | H | C₁₆H₁₇FN₂O | 273.13 | 273.2 |

1 2-Chloro-3-methyl-4-((R)-phenyl-(S)-pyrrolidin-3-ylmethoxy)pyridine
2 2,6-Dichloro-4-((R)-phenyl-(S)-pyrrolidin-3-ylmethoxy)pyridine
3 2-Chloro-4-((R)-phenyl-pyrrolidin-3-yl-methoxy)pyridine
4 4-((R)-Phenyl-(S)-pyrrolidin-3-yl-methoxy)pyridine-2-carbonitrile
5 2-Fluoro-4-(S)-phenyl-(R)-pyrrolidin-3-ylmethoxy)pyridine
6 2-Fluoro-4-(R)-phenyl-(S)-pyrrolidin-3-ylmethoxy)pyridine

### Example 17

### 5-Chloro-2-[(R)-(5-fluoropyridin-3-yl)-(S)-pyrrolidin-3-yl-methoxy]-3-methylpyridine

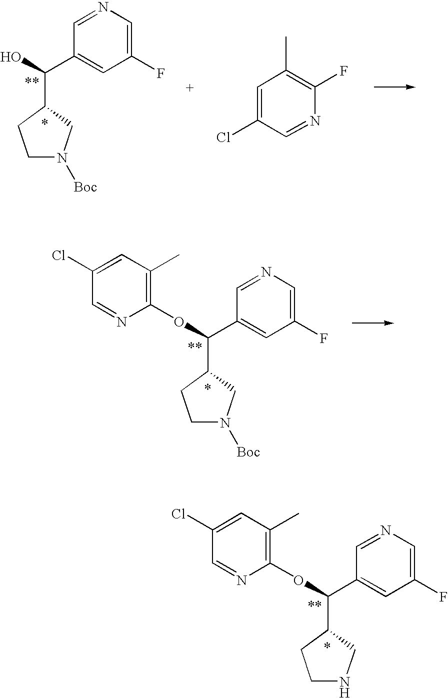

(S)-3-[(R)-(5-Fluoropyridin-3-yl)hydroxymethyl]pyrrolidine-1-carboxylic acid t-butyl ester (40 mg, 0.1 mmol) was dissolved in DMF (540 μL, 7.0 mmol). Washed and dried sodium hydride (4.3 mg, 179 μmol) was added and the resulting mixture was stirred at room temperature for 15 minutes. To this was added 5-chloro-2-fluoro-3-picoline (32.6 mg, 224 μmol) and the mixture was stirred at 70° C. for 3 hours. The mixture was concentrated to yield (S)-3-[(R)-(5-Chloro-3-methylpyridin-2-yloxy)-(5-fluoropyridin-3-yl)methyl]pyrrolidine-1-carboxylic acid t-butyl ester as an oil, which was used in the next step without further purification.

The oily residue from the previous step was dissolved in 1.25 M HCl in EtOH (835 μL, 1.0 mmol) and stirred at room temperature for 18 hours, and then evaporated to dryness. The residue was purified by reverse phase preparative HPLC to afford the TFA salt of the title compound (25.6 mg, 59% yield, 100% pure). MS m/z: [M+H]⁺ calcd for C₁₆H₁₇ClFN₃O, 322.1; found, 322.0.

### Example 18

Following the procedures described in the examples above or in the Schemes, and substituting the appropriate starting materials and reagents, compounds 1 and 2, having the following formula was also prepared as a TFA salt:

(IV-a)

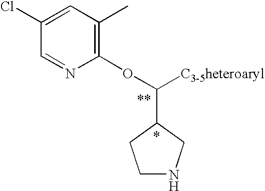

| | | | | | | | MS m/z: | |
|---|---|---|---|---|---|---|---|---|
| | | | | | | | [M + H]⁺ | |
| # | * | ** | R^{1a} | R^{1c} | C₃₋₅heteroaryl | Formula | calcd | found |
| 1 | S | R | Me | Cl | 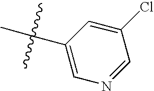 | C₁₆H₁₇Cl₂N₃O | 338.08 | 338.0 |
| 2 | S | R | Me | Cl | 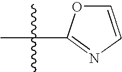 | C₁₄H₁₆ClN₃O₂ | 294.09 | 294.0 |

1 5-Chloro-2-[(R)-(5-chloropyridin-3-yl)-(S)-pyrrolidin-3-yl-methoxy]-3-methyl-pyridine
2 5-Chloro-3-methyl-2-((R)-oxazol-2-yl-(S)-pyrrolidin-3-yl-methoxy)pyridine

### Example 19

Following the procedures described in the examples above or in the Schemes, and substituting the appropriate starting materials and reagents, compounds 1 to 3, having the following formula were also prepared as TFA salts:

(V-a)

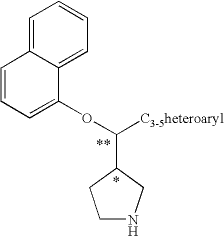

| | | | | | MS m/z: | |
|---|---|---|---|---|---|---|
| | | | | | [M + H]⁺ | |
| # | * | ** | —C₃₋₃heteroaryl | Formula | calcd | found |
| 1 | S | R | 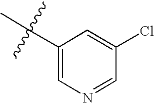 | C₂₀H₁₉ClN₂O | 339.12 | 339.0 |
| 2 | S | R | 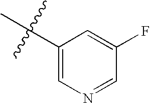 | C₂₀H₁₉FN₂O | 323.15 | 323.2 |
| 3 | S | R | 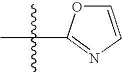 | C₂₀H₁₈N₂O₂ | 295.14 | 295.0 |

1 3-Chloro-5-[(R)-(naphthalen-1-yloxy)-(S)-pyrrolidin-3-yl-methyl]
pyridine
2 3-Fluoro-5-[(R)-(naphthalen-1-yloxy)-(S)-pyrrolidin-3-yl-methyl]
pyridine
3 2-[(R)-(Naphthalen-1-yloxy)-(S)-pyrrolidin-3-yl-methyl]oxazole

### Example 20

Following the procedures described in the examples above or in the Schemes, and substituting the appropriate starting materials and reagents, compounds 1 to 3, having the following formula were also prepared as TFA salts:

(V-b)

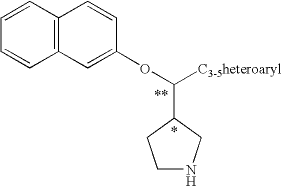

| | | | | | MS m/z: | |
|---|---|---|---|---|---|---|
| | | | | | [M + H]⁺ | |
| # | * | ** | C₃₋₅heteroaryl | Formula | calcd | found |
| 1 | S | R | 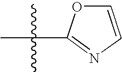 | C₁₉H₁₈N₂O₂ | 295.14 | 295.0 |
| 2 | S | R | 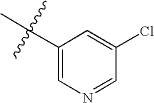 | C₂₀H₁₉ClN₂O | 339.12 | 339.0 |
| 3 | S | R | 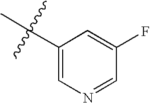 | C₂₀H₁₉FN₂O | 323.15 | 323.2 |

1 2-[(R)-(Naphthalen-2-yloxy)-(S)-pyrrolidin-3-yl-methyl]oxazole
2 3-Chloro-5-[(R)-(naphthalen-2-yloxy)-(S)-pyrrolidin-3-yl-methyl]
pyridine
3 3-Fluoro-5-[(R)-(naphthalen-2-yloxy)-(S)-pyrrolidin-3-yl-methyl]
pyridine

### Example 21

Following the procedures described in the examples above or in the Schemes, and substituting the appropriate starting materials and reagents, compounds 1 to 9, having the following formula were also prepared, compound 1 as a HCl salt and compounds 2-9 as TFA salts:

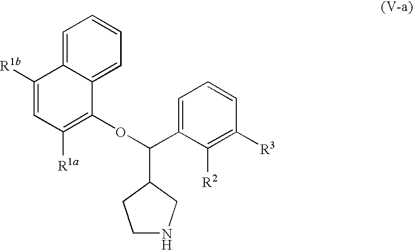

A diastereomeric mixture of the protected alcohol intermediate was purified by reverse phase preparative HPLC to obtain the RS/SR mixture of enantiomers and the SS/RR mixture of enantiomers. The RS/SR mixture of enantiomers was then used to obtain the following compounds.

| | | | | | | | | MS m/z: | |
|---|---|---|---|---|---|---|---|---|---|
| | | | | | | | | [M + H]⁺ | |
| # | * | ** | R^{1a} | R^{1b} | R² | R³ | Formula | calcd | found |

| | | | | | | | | |
|---|---|---|---|---|---|---|---|---|
| 1 | RS/SR | H | H | H | H | C₂₁H₂₁NO | 304.16 | 304.2 |
| 2 | SR/RS | H | H | H | H | C₂₁H₂₁NO | 304.16 | 304.2 |
| 3 | RS/SR | H | H | Me | H | C₂₂H₂₃NO | 318.18 | 318.2 |
| 4 | RS/SR | H | H | H | F | C₂₁H₂₀FNO | 322.15 | 322.2 |
| 5 | RS/SR | H | H | H | Cl | C₂₁H₂₀ClNO | 338.12 | 338.2 |
| 6 | RS/SR | H | H | H | OMe | C₂₂H₂₃NO₂ | 334.17 | 334.2 |
| 7 | RS/SR | H | H | H | —OCF₃ | C₂₂H₂₀F₃NO₂ | 388.14 | 388.2 |
| 8 | RS/SR | OMe | H | H | H | C₂₂H₂₃NO₂ | 334.17 | 334.2 |
| 9 | RS/SR | H | F | H | H | C₂₁H₂₀FNO | 322.15 | 322.2 |

1. 3-[(Naphthalen-1-yloxy)phenylmethyl]pyrrolidine
2. 3-[(Naphthalen-1-yloxy)phenylmethyl]pyrrolidine
3. 3-[(Naphthalen-1-yloxy)-o-tolylmethyl]pyrrolidine
4. 3-[(3-Fluorophenyl)(naphthalen-1-yloxy)-methyl]pyrrolidine
5. 3-[(3-Chlorophenyl)(naphthalen-1-yloxy)methyl]pyrrolidine
6. 3-[(3-Methoxyphenyl)(naphthalen-1-yloxy)methyl]pyrrolidine
7. 3-[(Naphthalen-1-yloxy)(3-trifluoromethoxyphenyl)methyl]pyrrolidine
8. 3-[(2-Methoxynaphthalen-1-yloxy)phenylmethyl]pyrrolidine
9. 3-[(R)-(4-Fluoronaphthalen-1-yloxy)phenylmethyl]pyrrolidine

### Assay 1

### hSERT, hNET, and hDAT Binding Assays

Membrane radioligand binding assays were used to measure inhibition of labeled ligand (³H-citalopram or ³H-nisoxetine or ³H-WIN35428) binding to membranes prepared from cells expressing the respective human recombinant transporter (hSERT or hNET or hDAT) in order to determine the pKᵢ values of test compounds at the transporters.

### Membrane Preparation From Cells Expressing hSERT, hNET, or hDAT

Recombinant human embryonic kidney (HEK-293) derived cell lines stably transfected with hSERT or hNET, respectively, were grown in DMEM medium supplemented with 10% dialyzed FBS (for hSERT) or FBS (for hNET), 100 μg/ml penicillin, 100 μg/ml streptomycin, 2 mM L-glutamine and 250 μg/ml of the aminoglycoside antibiotic G418, in a 5% CO₂ humidified incubator at 37° C. When cultures reached 80% confluence, the cells were washed thoroughly in PBS (without Ca¹⁺ and Mg²⁺) and lifted with 5 mM EDTA in PBS. Cells were pelleted by centrifugation, resuspended in lysis buffer (10 mM Tris-HCl, pH7.5 containing 1 mM EDTA), homogenized, pelleted by centrifugation, then resuspended in 50 mM Tris-HCl, pH 7.5 and 10% sucrose at 4° C. Protein concentration of the membrane suspension was determined using a Bio-Rad Bradford Protein Assay kit. Membranes were snap frozen and stored at −80° C. Chinese hamster ovary membranes expressing hDAT (CHO-DAT) were purchased from PerkinElmer and stored at −80° C.

### Binding Assays

Binding assays were performed in a 96-well assay plate in a total volume of 200 μl assay buffer (50 mM Tris-HCl, 120 mM NaCl, 5 mM KCl, pH 7.4) with 0.5, 1, and 3 μg membrane protein, for SERT, NET and DAT, respectively. Saturation binding studies, to determine radioligand K_{d} values for ³H-citalopram, ³H-nisoxetine, or ³H-WIN35428, respectively were conducted using 12 different radioligand concentrations ranging from 0.005-10 nM (³H-citalopram); 0.01-20 nM (³H-nisoxetine) and 0.2-50 nM (³H-WIN35428). Inhibition assays for determination of pKᵢ values of test compounds were conducted with 1.0 nM ³H-citalopram, 1.0 nM ³H-nisoxetine or 3.0 nM ³H-WIN35428, at 11 different concentrations of test compound ranging from 10 pM to 100 μM.

Stock solutions (10 mM in DMSO) of test compound were prepared and serial dilutions made using Dilution Buffer (50 mM Tris-HCl, 120 mM NaCl, 5 mM KCl, pH 7.4, 0.1% BSA, 400 μM ascorbic acid). Non-specific radioligand binding was determined in the presence of 1 μM duloxetine, 1 μM desipramine or 10 μM GBR12909 (each in Dilution Buffer) for the hSERT, hNET or hDAT assays, respectively.

Following a 60 minute incubation at 22° C. (or a period sufficient to reach equilibrium), the membranes were harvested by rapid filtration over a 96-well UniFilter GF/B plate, pretreated with 0.3% polyethyleneimine, and washed 6 times with 300 μl wash buffer (50 mM Tris-HCl, 0.9% NaCl, pH 7.5 at 4° C.). Plates were dried overnight at room temperature, ˜45 μl of MicroScint™-20 (Perkin Elmer) added and bound radioactivity quantitated via liquid scintillation spectroscopy. Inhibition curves and saturation isotherms were analyzed using GraphPad Prism Software package (GraphPad Software, Inc., San Diego, Calif.). IC₅₀ values were generated from concentration response curves using the Sigmoidal Dose Response (variable slope) algorithm in Prism GraphPad. K_{d} and Bₘₐₓ values for the radioligand were generated from saturation isotherms using the Saturation Binding Global Fit algorithm in Prism GraphPad. pKᵢ (negative decadic logarithm of Kᵢ) values for test compounds were calculated from the best-fit IC₅₀ values, and the K_{d} value of the radioligand, using the Cheng-Prusoff equation (Cheng & Prusoff (1973) Biochem. Pharmacol. 22(23):3099-3108): Kᵢ=IC₅₀/(1+[L]/K_{d}), where [L]=concentration radioligand.

All the aforementioned compounds were tested in this assay and found to exhibit a SERT pKᵢ≧5.0 and/or a NET pKᵢ≧5.0, with numerous compounds exhibiting a SERT pKᵢ≧7.0 and/or NET pKᵢ≧7.0.

| | SERT | NET |
|---|---|---|
| Ex. | pKᵢ | pKᵢ |
| 1 | ≧7.0 | ≧7.0 |
| 2-1 | ≧7.0 | ≧5.0 |
| 2-2 | ≧7.0 | ≧7.0 |
| 2-3 | ≧7.0 | ≧7.0 |
| 2-4 | ≧7.0 | ≧5.0 |
| 2-5 | ≧7.0 | ≧7.0 |
| 2-6 | ≧7.0 | ≧7.0 |
| 2-7 | ≧7.0 | ≧5.0 |
| 2-8 | ≧7.0 | ≧5.0 |
| 2-9 | ≧7.0 | ≧7.0 |
| 2-10 | ≧7.0 | ≧7.0 |
| 2-11 | ≧7.0 | ≧7.0 |
| 2-12 | ≧7.0 | ≧7.0 |
| 2-13 | ≧5.0 | ≧5.0 |
| 2-14 | ≧7.0 | ≧7.0 |
| 2-15 | ≧7.0 | ≧7.0 |
| 2-16 | ≧7.0 | ≧7.0 |
| 2-17 | ≧7.0 | ≧7.0 |
| 2-18 | ≧7.0 | ≧7.0 |
| 2-19 | ≧7.0 | ≧7.0 |
| 2-20 | ≧7.0 | ≧7.0 |
| 2-21 | ≧7.0 | ≧7.0 |
| 2-22 | ≧7.0 | ≧7.0 |
| 2-23 | ≧7.0 | ≧7.0 |
| 2-24 | ≧7.0 | ≧7.0 |
| 2-25 | ≧7.0 | ≧7.0 |
| 2-26 | ≧7.0 | ≧5.0 |
| 2-27 | ≧7.0 | ≧7.0 |
| 2-28 | ≧7.0 | ≧7.0 |
| 2-29 | ≧7.0 | ≧5.0 |
| 2-30 | ≧7.0 | ≧5.0 |
| 2-31 | ≧7.0 | ≧7.0 |
| 2-32 | ≧7.0 | ≧7.0 |
| 2-33 | ≧7.0 | ≧7.0 |
| 2-34 | ≧7.0 | ≧5.0 |
| 2-35 | ≧7.0 | ≧7.0 |
| 2-36 | ≧7.0 | ≧7.0 |
| 2-37 | ≧7.0 | ≧7.0 |
| 2-38 | ≧7.0 | ≧7.0 |
| 2-39 | ≧7.0 | ≧5.0 |
| 2-40 | ≧7.0 | ≧7.0 |
| 2-41 | ≧7.0 | ≧7.0 |
| 2-42 | ≧7.0 | ≧5.0 |
| 2-43 | ≧7.0 | ≧7.0 |
| 2-44 | ≧7.0 | ≧7.0 |
| 2-45 | ≧7.0 | ≧7.0 |
| 2-46 | ≧7.0 | ≧5.0 |
| 2-47 | ≧7.0 | ≧7.0 |
| 2-48 | ≧7.0 | ≧7.0 |
| 2-49 | ≧7.0 | ≧7.0 |
| 2-50 | ≧7.0 | ≧5.0 |
| 2-51 | ≧7.0 | ≧7.0 |
| 2-52 | ≧7.0 | ≧7.0 |
| 2-53 | ≧7.0 | ≧7.0 |
| 2-54 | ≧7.0 | ≧5.0 |
| 2-55 | ≧5.0 | ≧7.0 |
| 2-56 | ≧7.0 | ≧7.0 |
| 2-57 | ≧7.0 | ≧7.0 |
| 2-58 | ≧7.0 | ≧7.0 |
| 2-59 | ≧7.0 | ≧7.0 |
| 2-60 | ≧7.0 | ≧7.0 |
| 2-61 | ≧7.0 | ≧5.0 |
| 2-62 | ≧5.0 | ≧7.0 |
| 2-63 | ≧5.0 | ≧5.0 |
| 2-64 | ≧7.0 | ≧7.0 |
| 2-65 | ≧7.0 | ≧7.0 |
| 2-66 | ≧7.0 | ≧7.0 |
| 2-67 | ≧7.0 | ≧7.0 |
| 2-68 | ≧5.0 | ≧5.0 |
| 2-69 | ≧7.0 | ≧7.0 |
| 2-70 | ≧5.0 | ≧5.0 |
| 2-71 | ≧7.0 | ≧7.0 |
| 2-72 | ≧5.0 | ≧5.0 |
| 2-73 | ≧7.0 | ≧7.0 |
| 2-74 | ≧7.0 | ≧7.0 |
| 2-75 | ≧7.0 | ≧7.0 |
| 2-76 | ≧7.0 | ≧7.0 |
| 2-77 | ≧7.0 | ≧7.0 |
| 2-78 | ≧7.0 | ≧7.0 |
| 2-79 | ≧7.0 | ≧7.0 |
| 2-80 | ≧7.0 | ≧7.0 |
| 2-81 | ≧5.0 | ≧7.0 |
| 2-82 | ≧7.0 | ≧7.0 |
| 3-1 | ≧5.0 | ≧5.0 |
| 3-2 | ≧7.0 | ≧7.0 |
| 3-3 | ≧5.0 | ≧5.0 |
| 3-4 | ≧5.0 | ≧5.0 |
| 3-5 | ≧7.0 | ≧7.0 |
| 3-6 | ≧7.0 | ≧5.0 |
| 3-7 | ≧7.0 | ≧7.0 |
| 4 | ≧7.0 | ≧7.0 |
| 5-1 | ≧7.0 | ≧5.0 |
| 5-2 | ≧7.0 | ≧7.0 |
| 5-3 | ≧5.0 | ≧5.0 |
| 5-4 | ≧5.0 | ≧7.0 |
| 5-5 | ≧7.0 | ≧7.0 |
| 5-6 | ≧5.0 | ≧5.0 |
| 5-7 | ≧7.0 | ≧7.0 |
| 5-8 | ≧5.0 | ≧5.0 |
| 5-9 | ≧7.0 | ≧7.0 |
| 5-10 | ≧5.0 | ≧5.0 |
| 5-11 | ≧7.0 | ≧5.0 |
| 5-12 | ≧7.0 | ≧5.0 |
| 5-13 | ≧5.0 | ≧5.0 |
| 5-14 | ≧7.0 | ≧5.0 |
| 5-15 | ≧7.0 | ≧5.0 |
| 5-16 | ≧7.0 | ≧7.0 |
| 5-17 | ≧7.0 | ≧7.0 |
| 5-18 | ≧7.0 | ≧7.0 |
| 5-19 | ≧7.0 | ≧5.0 |
| 5-20 | ≧7.0 | ≧7.0 |
| 5-21 | ≧7.0 | ≧7.0 |
| 5-22 | ≧7.0 | ≧7.0 |
| 5-23 | ≧7.0 | ≧7.0 |
| 5-24 | ≧7.0 | ≧5.0 |
| 5-25 | ≧7.0 | ≧7.0 |
| 5-26 | ≧7.0 | ≧7.0 |
| 5-27 | ≧7.0 | ≧7.0 |
| 5-28 | ≧7.0 | ≧5.0 |
| 5-29 | ≧7.0 | ≧5.0 |
| 5-30 | ≧7.0 | ≧5.0 |
| 5-31 | ≧7.0 | ≧7.0 |
| 5-32 | ≧7.0 | ≧5.0 |
| 5-33 | ≧7.0 | ≧7.0 |
| 6-1 | ≧7.0 | ≧7.0 |
| 6-2 | ≧5.0 | ≧5.0 |
| 6-3 | ≧7.0 | ≧7.0 |
| 6-4 | ≧7.0 | ≧7.0 |
| 6-5 | ≧7.0 | ≧7.0 |
| 6-6 | ≧7.0 | ≧5.0 |
| 6-7 | ≧5.0 | ≧7.0 |
| 6-8 | ≧7.0 | ≧7.0 |
| 6-9 | ≧5.0 | ≧7.0 |
| 6-10 | ≧7.0 | ≧5.0 |
| 6-11 | ≧7.0 | ≧5.0 |
| 6-12 | ≧7.0 | ≧5.0 |
| 6-13 | ≧7.0 | ≧7.0 |
| 6-14 | ≧7.0 | ≧7.0 |
| 6-15 | ≧7.0 | ≧5.0 |
| 6-16 | ≧7.0 | ≧7.0 |
| 6-17 | ≧5.0 | ≧7.0 |
| 6-18 | ≧7.0 | ≧7.0 |
| 6-19 | ≧5.0 | ≧7.0 |
| 6-20 | ≧5.0 | ≧7.0 |
| 6-21 | ≧5.0 | ≧5.0 |
| 6-22 | ≧5.0 | ≧7.0 |
| 6-23 | ≧7.0 | ≧7.0 |
| 6-24 | ≧7.0 | ≧7.0 |
| 6-25 | ≧5.0 | ≧7.0 |
| 6-26 | ≧7.0 | ≧7.0 |
| 6-27 | ≧7.0 | ~5.0* |
| 6-28 | ≧5.0 | ≧7.0 |
| 6-29 | ≧7.0 | ≧7.0 |
| 6-30 | ≧7.0 | ≧7.0 |
| 6-31 | ≧7.0 | ≧7.0 |
| 6-32 | ≧5.0 | ≧7.0 |
| 6-33 | ≧5.0 | ≧7.0 |
| 6-34 | ≧7.0 | ≧7.0 |
| 6-35 | ≧7.0 | ≧5.0 |
| 6-36 | ≧5.0 | ≧7.0 |
| 6-37 | ≧5.0 | ≧7.0 |
| 6-38 | ≧5.0 | ≧7.0 |
| 6-39 | ≧7.0 | ≧7.0 |
| 6-40 | ≧7.0 | ≧7.0 |
| 6-41 | ≧5.0 | ≧7.0 |
| 6-42 | ≧5.0 | ≧7.0 |
| 6-43 | ≧7.0 | ≧7.0 |
| 6-44 | ≧7.0 | ≧7.0 |
| 6-45 | ≧5.0 | ≧7.0 |
| 6-46 | ≧5.0 | ≧5.0 |
| 6-47 | ≧7.0 | ≧7.0 |
| 6-48 | ≧7.0 | ≧7.0 |
| 6-49 | ≧7.0 | ≧7.0 |
| 6-50 | ≧7.0 | ≧7.0 |
| 6-51 | ≧7.0 | ≧7.0 |
| 6-52 | ≧5.0 | ≧7.0 |
| 6-53 | ≧7.0 | ≧7.0 |
| 7-1 | ≧5.0 | ≧5.0 |
| 7-2 | ≧7.0 | ≧7.0 |
| 7-3 | ≧7.0 | ≧7.0 |
| 8-1 | ≧5.0 | ≧5.0 |
| 8-2 | ≧5.0 | ≧5.0 |
| 8-3 | ≧7.0 | ≧7.0 |
| 8-4 | ≧7.0 | ≧7.0 |
| 9-1 | ≧7.0 | ≧7.0 |
| 10-1 | ≧7.0 | ≧7.0 |
| 10-2 | ≧5.0 | ≧5.0 |
| 10-3 | ≧5.0 | ≧7.0 |
| 10-4 | ≧7.0 | ≧5.0 |
| 10-5 | ≧7.0 | ≧7.0 |
| 10-6 | ≧7.0 | ≧5.0 |
| 10-7 | ≧5.0 | ≧7.0 |
| 10-8 | ≧5.0 | ≧5.0 |
| 10-9 | ≧7.0 | ≧7.0 |
| 10-10 | ≧7.0 | ≧5.0 |
| 11-1 | ≧7.0 | ≧7.0 |
| 11-2 | ≧7.0 | ≧5.0 |
| 11-3 | ≧5.0 | ≧7.0 |
| 11-4 | ≧5.0 | ≧5.0 |
| 11-5 | ≧7.0 | ≧5.0 |
| 12-1 | ≧5.0 | ≧7.0 |
| 12-2 | ≧7.0 | ≧5.0 |
| 12-3 | ≧7.0 | ≧7.0 |
| 12-4 | ≧5.0 | ≧7.0 |
| 12-5 | ≧7.0 | ≧5.0 |
| 13 | ≧7.0 | ≧7.0 |
| 14-1 | ≧7.0 | ≧5.0 |
| 14-2 | ≧5.0 | ≧7.0 |
| 14-3 | ≧7.0 | ≧5.0 |
| 14-4 | ≧7.0 | ≧5.0 |
| 14-5 | ≧5.0 | ≧7.0 |
| 14-6 | ≧7.0 | ≧7.0 |
| 14-7 | ≧7.0 | ≧5.0 |
| 14-8 | ≧7.0 | ≧7.0 |
| 14-9 | ≧7.0 | ≧7.0 |
| 14-10 | ≧5.0 | ≧7.0 |
| 14-11 | ≧5.0 | ≧5.0 |
| 14-12 | ≧7.0 | ≧7.0 |
| 14-13 | ≧7.0 | ≧7.0 |
| 14-14 | ≧7.0 | ≧7.0 |
| 14-15 | ≧7.0 | ≧7.0 |
| 14-16 | ≧7.0 | ≧7.0 |
| 14-17 | ≧5.0 | ≧7.0 |
| 14-18 | ≧7.0 | ≧7.0 |
| 14-19 | ≧7.0 | ≧5.0 |
| 14-20 | ≧7.0 | ≧7.0 |
| 14-21 | ≧7.0 | ≧7.0 |
| 14-22 | ≧7.0 | ≧7.0 |
| 14-23 | ≧7.0 | ≧5.0 |
| 14-24 | ≧7.0 | ≧5.0 |
| 14-25 | ≧7.0 | ≧5.0 |
| 14-26 | ≧7.0 | ≧7.0 |
| 14-27 | ≧7.0 | ≧7.0 |
| 14-28 | ≧7.0 | ≧5.0 |
| 14-29 | ≧7.0 | ≧7.0 |
| 14-30 | ≧5.0 | ≧7.0 |
| 14-31 | ≧7.0 | ≧7.0 |
| 14-32 | ≧7.0 | ≧7.0 |
| 14-33 | ≧5.0 | ≧5.0 |
| 14-34 | ≧7.0 | ≧5.0 |
| 14-35 | ≧5.0 | ≧7.0 |
| 14-36 | ≧7.0 | ≧5.0 |
| 14-37 | ≧5.0 | ≧5.0 |
| 14-38 | ≧5.0 | ≧7.0 |
| 14-39 | ≧7.0 | ≧5.0 |
| 14-40 | ≧5.0 | ≧7.0 |
| 14-41 | ≧7.0 | ≧7.0 |
| 14-42 | ≧5.0 | ≧5.0 |
| 14-43 | ≧7.0 | ≧7.0 |
| 15-1 | ≧7.0 | ≧7.0 |
| 15-2 | ≧7.0 | ≧5.0 |
| 15-3 | ≧7.0 | ≧7.0 |
| 15-4 | ≧7.0 | ≧7.0 |
| 15-5 | ≧7.0 | ≧7.0 |
| 15-6 | ≧7.0 | ≧7.0 |
| 15-7 | ≧7.0 | ≧5.0 |
| 15-8 | ≧7.0 | ≧7.0 |
| 15-9 | ≧5.0 | ≧5.0 |
| 15-10 | ≧7.0 | ≧7.0 |
| 15-11 | ≧7.0 | ≧7.0 |
| 15-12 | ≧7.0 | ≧7.0 |
| 15-13 | ≧7.0 | ≧7.0 |
| 15-14 | ≧5.0 | ≧7.0 |
| 15-15 | ≧5.0 | ≧7.0 |
| 15-16 | ≧7.0 | ≧7.0 |
| 15-17 | ≧7.0 | ≧7.0 |
| 15-18 | ≧7.0 | ≧7.0 |
| 15-19 | ≧7.0 | ≧7.0 |
| 15-20 | ≧7.0 | ≧7.0 |
| 15-21 | ≧5.0 | ≧7.0 |
| 15-22 | ≧7.0 | ≧5.0 |
| 15-23 | ≧7.0 | ≧5.0 |
| 15-24 | ≧7.0 | ≧5.0 |
| 15-25 | ≧7.0 | ≧5.0 |
| 15-26 | ≧7.0 | ≧5.0 |
| 15-27 | ≧7.0 | ≧5.0 |
| 15-28 | ≧7.0 | ≧5.0 |
| 15-29 | ≧7.0 | ≧5.0 |
| 15-30 | ≧7.0 | ≧5.0 |
| 15-31 | ≧7.0 | ≧5.0 |
| 15-32 | ≧7.0 | ≧5.0 |
| 16-1 | ≧7.0 | ≧5.0 |
| 16-2 | ≧7.0 | ≧5.0 |
| 16-3 | ≧7.0 | ≧5.0 |
| 16-4 | ≧5.0 | ≧5.0 |
| 16-5 | ≧7.0 | ≧5.0 |
| 16-6 | ≧5.0 | ≧5.0 |
| 17 | ≧7.0 | ≧7.0 |
| 18-1 | ≧7.0 | ≧7.0 |
| 18-2 | ≧7.0 | ≧7.0 |
| 19-1 | ≧7.0 | ≧7.0 |
| 19-2 | ≧7.0 | ≧7.0 |
| 19-3 | ≧7.0 | ≧7.0 |
| 20-1 | ≧7.0 | ≧7.0 |
| 20-2 | ≧7.0 | ≧7.0 |
| 20-3 | ≧7.0 | ≧7.0 |
| 21-1 | ≧7.0 | ≧7.0 |
| 21-2 | ≧7.0 | ≧7.0 |
| 21-3 | ≧7.0 | ≧7.0 |
| 21-4 | ≧7.0 | ≧7.0 |
| 21-5 | ≧7.0 | ≧7.0 |
| 21-6 | ≧7.0 | ≧7.0 |
| 21-7 | ≧7.0 | ≧5.0 |
| 21-8 | ≧7.0 | ≧7.0 |
| 21-9 | ≧7.0 | ≧7.0 |

*This value was determined to be slightly less than 5.0

### Assay 2

### hSERT and hNET Neurotransmitter Uptake Assays

Neurotransmitter uptake assays were used to measure inhibition of ³H-serotonin (³H-5-HT) and ³H-norepinephrine (³H-NE) uptake into cells expressing the respective transporter (hSERT or hNET) in order to determine the pIC₅₀ values of test compounds at the transporters.

### ³H-5-HT and ³H-NE Uptake Assays

HEK-293 derived cell lines stably-transfected with hSERT or hNET, respectively, were grown in DMEM medium supplemented with 10% dialyzed FBS (for hSERT) or FBS (for hNET), 100 μg/ml penicillin, 100 μg/ml streptomycin, 2 mM L-glutamine and 250 μg/ml of the aminoglycoside antibiotic G418, in a 5% CO₂ humidified incubator at 37° C. When cultures reached 80% confluence, the cells were washed thoroughly in PBS (without Ca²⁺ and Mg²⁺) and lifted with 5 mM EDTA in PBS. Cells were harvested by centrifugation at 1100 rpm for 5 minutes, washed once by resuspension in PBS, then centrifuged. The supernatant was discarded and the cell pellet resuspended, by gentle trituration, in room temperature Krebs-Ringer bicarbonate buffer containing HEPES (10 mM), CaCl₂ (2.2 mM), ascorbic acid (200 μM) and pargyline (200 μM), pH 7.4. The final concentration of cells in the cell suspension was 7.5×10⁴ cells/ml and 1.25×10⁵ cells/ml for SERT and NET cell lines, respectively.

Neurotransmitter uptake assays were performed in a 96-well assay plate in a total volume of 400 μl assay buffer (Krebs-Ringer bicarbonate buffer containing HEPES (10 mM), CaCl₂ (2.2 mM), ascorbic acid (200 μM) and pargyline (200 μM), pH 7.4) with 1.5×10⁴ and 2.5×10⁴ cells, for SERT and NET, respectively. Inhibition assays for determination of pIC₅₀ values of test compounds were conducted with 11 different concentrations, ranging from 10 pM to 100 μM. Stock solutions (10 mM in DMSO) of test compound were prepared and serial dilutions prepared using 50 mM Tris-HCl, 120 mM NaCl, 5 mM KCl, pH 7.4, 0.1% BSA, 400 μM ascorbic acid. Test compounds were incubated for 30 minutes at 37° C. with the respective cells, prior to addition of radiolabeled neurotransmitter, ³H-5-HT (20 nM final concentration) or ³H-NE (50 nM final concentration). Non-specific neurotransmitter uptake was determined in the presence of 2.5 μM duloxetine or 2.5 μM desipramine (each in Dilution Buffer) for the hSERT or hNET assays, respectively.

Following a 10 minute incubation, at 37° C., with radioligand, the cells were harvested by rapid filtration over a 96-well UniFilter GF/B plate, pretreated with 1% BSA, and washed 6 times with 650 μl wash buffer (ice cold PBS). Plates were dried overnight at 37° C., ˜45 μl of MicroScint™-20 (Perkin Elmer) added and incorporated radioactivity quantitated via liquid scintillation spectroscopy Inhibition curves were analyzed using GraphPad Prism Software package (GraphPad Software, Inc., San Diego, Calif.). IC₅₀ values were generated from concentration response curves using the Sigmoidal Dose Response (variable slope) algorithm in Prism GraphPad.

### Assay 3

### Ex Vivo SERT and NET Transporter Occupancy Studies

Ex vivo radioligand binding and neurotransmitter uptake assays are used to determine the in vivo occupancy of SERT and NET, in selected brain regions, following in vivo administration (acute or chronic) of test compounds. Following administration of test compound (by intravenous, intraperitoneal, oral, subcutaneous or other route) at the appropriate dose (0.0001 to 100 mg/kg), rats (≧n=4 per group) are euthanized at specific time points (10 minutes to 48 hours) by decapitation and the brain dissected on ice. Relevant brain regions are dissected, frozen and stored at −80° C. until use.

### Ex Vivo SERT and NET Radioligand Binding Assays

For ex vivo radioligand binding assays, the initial rates of association of SERT (³H-citalopram), and NET (³H-nisoxetine) selective radioligands with rat brain crude homogenates, prepared from vehicle and test compound-treated animals, are monitored (see Hess et al. (2004) J. Pharmacol. Exp. Ther. 310(2):488-497). Crude brain tissue homogenates are prepared by homogenizing frozen tissue pieces in 0.15 ml (per mg wet weight) of 50 mM Tris-HCl, 120 mM NaCl, 5 mM KCl, pH 7.4 buffer. Radioligand association assays are performed in a 96-well assay plate in a total volume of 200 μl assay buffer (50 mM Tris-HCl, 120 mM NaCl, 5 mM KCl, 0.025% BSA, pH 7.4) with 650 μg wet weight tissue (equivalent to 25 μg protein). Homogenates are incubated for up to 5 minutes with ³H-citalopram (3 nM) and ³H-nisoxetine (5 nM), respectively, prior to termination of the assay by rapid filtration over a 96-well UniFilter GF/B plate, pretreated with 0.3% polyethyleneimine. Filters then are washed 6 times with 300 μl wash buffer (50 mM Tris-HCl, 0.9% NaCl, pH 7.4 at 4° C.). Non-specific radioligand binding is determined in the presence of 1 μM duloxetine, or 1 μM despiramine, for ³H-citalopram or ³H-nisoxetine, respectively. The plates are dried overnight at room temperature, ˜45 μl of MicroScint™-20 (Perkin Elmer) added and bound radioactivity quantitated via liquid scintillation spectroscopy. The initial rates of association of ³H-citalopram and ³H-nisoxetine are determined by linear regression using GraphPad Prism Software package (GraphPad Software, Inc., San Diego, Calif.). The average rate of radioligand association to brain tissue homogenates from vehicle-treated animals is determined. The % occupancy of test compounds then is determined using the following equation:

% occupancy=100×(1−(initial rate association for test compound-treated tissue/mean rate association for vehicle-treated tissue))

ED₅₀ values are determined by plotting the log 10 of the dose of the test compound against the % occupancy. ED₅₀ values are generated from concentration response curves using the Sigmoidal Dose Response (variable slope) algorithm in GraphPad Prism.

### Ex Vivo SERT and NET Uptake Assays

Ex vivo neurotransmitter uptake assays, in which the uptake of ³H-5-HT or ³H-NE into rat brain crude homogenates, prepared from vehicle and test compound-treated animals, are used to measure in vivo SERT and NET transporter occupancy (see Wong et al. (1993) Neuropsychopharmacology 8(1):23-33). Crude brain tissue homogenates are prepared by homogenizing frozen tissue pieces in 0.5 ml (per mg wet weight) of 10 mM HEPES buffer pH 7.4, containing 0.32 M sucrose, 200 μM ascorbic acid and 200 μM pargyline, at 22° C. Neurotransmitter uptake assays are performed in a 96-well Axygen plate in a total volume of 350 μl assay buffer (Krebs-Ringer bicarbonate buffer with 10 mM HEPES, 2.2 mM CaCl₂, 200 μM ascorbic acid and 200 μM pargyline, pH 7.4) with 50 μg protein. Homogenates are incubated for 5 minutes at 37° C. with ³H-5-HT (20 nM) and ³H-NE (50 nM), respectively, prior to termination of the assay by rapid filtration over a 96-well UniFilter GF/B plate, pretreated with 1% BSA. Plates are washed 6 times with 650 μl wash buffer (ice cold PBS) and dried overnight at 37° C., prior to addition of ˜45 μl of MicroScint™-20 (Perkin Elmer). Incorporated radioactivity is quantitated via liquid scintillation spectroscopy. Non-specific neurotransmitter uptake is determined in parallel assays in which tissue homogenates are incubated with ³H-5-HT (20 nM) or ³H-NE (50 nM) for 5 minutes at 4° C.

### Assay 4

### Other Assays

Other assays that were used to evaluate the pharmacological properties of test compounds include, but are not limited to, cold ligand binding kinetics assays (Motulsky and Mahan (1984) Molecular Pharmacol. 25(1):1-9) with membranes prepared from cells expressing hSERT or hNET; conventional membrane radioligand binding assays using radiolabeled, for example, tritiated, test compound; radioligand binding assays using native tissue from, for example rodent or human brain; neurotransmitter uptake assays using human or rodent platelets; neurotransmitter uptake assays using crude, or pure, synaptosome preparations from rodent brain.

### Assay 5

### Formalin Paw Test

Compounds are assessed for their ability to inhibit the behavioral response evoked by a 50 μl injection of formalin (5%). A metal band is affixed to the left hind paw of male Sprague-Dawley rats (200-250 g) and each rat is conditioned to the band for 60 minutes within a plastic cylinder (15 cm diameter). Compounds are prepared in pharmaceutically acceptable vehicles and administered systemically (i.p., p.o.) at pre-designated times before formalin challenge. Spontaneous nociceptive behaviors consisting of flinching of the injected (banded) hind paw are counted continuously for 60 minutes using an automated nociception analyzer (UCSD Anesthesiology Research, San Diego, Calif.). Antinociceptive properties of test articles are determined by comparing the number of flinches in the vehicle and compound-treated rats (Yaksh et al. (2001) J. Appl. Physiol. 90(6):2386-2402).

### Assay 6

### Spinal Nerve Ligation Model

Compounds are assessed for their ability to reverse tactile allodynia (increased sensitivity to an innocuous mechanical stimulus) induced by nerve injury. Male Sprague-Dawley rats are surgically prepared as described in Kim and Chung (1992) Pain 50(3):355-363. Mechanical sensitivity is determined as the 50% withdrawal response to innocuous mechanical stimuli (Chaplan et al. (1994) J. Neurosci. Methods 53(1):55-63) before and after nerve injury. One to four weeks post-surgery, compounds are prepared in pharmaceutically acceptable vehicles and administered systemically (i.p., p.o.). The degree of nerve injury-induced mechanical sensitivity before and after treatment serves as an index of the compounds' antinociceptive properties.

While the present invention has been described with reference to specific aspects or embodiments thereof, it will be understood by those of ordinary skilled in the art that various changes can be made or equivalents can be substituted without departing from the true spirit and scope of the invention. Additionally, to the extent permitted by applicable patent statues and regulations, all publications, patents and patent applications cited herein are hereby incorporated by reference in their entirety to the same extent as if each document had been individually incorporated by reference herein.

What is claimed is:

1. A compound of formula I: 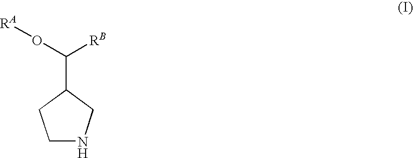
where: R^{A} and R^{B} are independently C₃₋₅heteroaryl, naphthalene, or 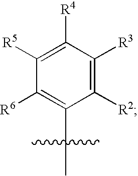
with the proviso that at least one of R^{A} and R^{B} is C₃₋₅heteroaryl or naphthalene; and further with the proviso that when R^{A} is: 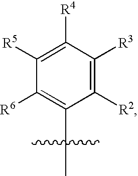
R^{B} is not unsubstituted 2-pyridine;
C₃₋₅heteroaryl and naphthalene are optionally substituted with one to four R¹ groups independently selected from halo, —C₁₋₆alkyl, —O—C₁₋₆alkyl, —O—C₃₋₇cycloalkyl, —CN, and —C₀₋₁alkylene-NR^{a}R^{b};
R² through R⁶ are independently selected from H, halo, —C₁₋₆alkyl, —C₂₋₆alkynyl, —O—C₁₋₆alkyl, —C₁₋₄alkylene-O—C₁₋₄alkyl, —C₃₋₇cycloalkyl, —C₀₋₁alkylene-phenyl, —O—C₀₋₃alkylene-phenyl, —C₀₋₆alkylene-OH, —CN, —C₀₋₂alkylene-COOH, —CHO, —C(O)—C₁₋₆alkyl, —C(O)O—C₁₋₄alkyl, —CH₂SH, —S—C₁₋₆alkyl, —C₁₋₄alkylene-S—C₁₋₄alkyl, —SO₂—C₁₋₆alkyl, —SO₂NR^{a}R^{b}, —NHSO₂R^{a}, —C₀₋₁alkylene-NR^{a}R^{b}, —NHC(O)—C₁₋₆alkyl, —C(O)NR^{a}R^{b}, and —NO_{2;} R^{a} and R^{b} are independently H or —C₁₋₄alkyl;
each alkyl in R¹ through R⁶ is optionally substituted with 1 to 5 fluoro atoms; and each phenyl in R² through R⁶ is optionally substituted with 1 or 2 groups independently selected from halo, —C₁₋₆alkyl, and —O—C₁₋₆alkyl;
or a pharmaceutically acceptable salt thereof.

2. The compound of claim 1, where each C₃₋₅heteroaryl group is independently selected from 2-pyridine, 3-pyridine, 4-pyridine, thiazole, oxazole, furan, pyrazine, and pyrimidine; and each pyridine is optionally substituted with one to two R¹ groups independently selected from halo, —C₁₋₆alkyl, —O—C₁₋₆alkyl, —O—C₃₋₇cycloalkyl, —CN, and —C₀₋₁alkylene-NR^{a}R^{b}, where R^{a} and R^{b} are —C₁₋₄alkyl.

3. The compound of claim 1, wherein R² is selected from H, halo, —C₁₋₆alkyl, —O—C₁₋₆alkyl, —C₃₋₇cycloalkyl, —C₀₋₁alkylene-phenyl, —O—C₀₋₃alkylene-phenyl, —C(O)—C₁₋₆alkyl, and —C(O)O—C₁₋₄alkyl, where each alkyl is optionally substituted with 1 to 5 fluoro atoms.

4. The compound of claim 1, wherein R³ is selected from H, halo, and —C₁₋₆alkyl.

5. The compound of claim 1, wherein R⁴ is selected from H, halo, —C₁₋₆alkyl, —O—C₁₋₆alkyl, and —C₀₋₁alkylene-phenyl.

6. The compound of claim 1, wherein R⁵ is selected from H, halo, and —C₁₋₆alkyl.

7. The compound of claim 1, wherein R⁶ is selected from H, halo, and —C₁₋₆alkyl.

8. The compound of claim 1, wherein R^{A} and R^{B} are independently selected from 2-pyridine, 3-pyridine, 4-pyridine, thiazole, oxazole, furan, pyrazine, pyrimidine, naphthalene, and 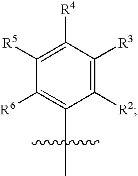
where each pyridine and naphthalene group is optionally substituted with one to four R¹ groups independently selected from halo, —C₁₋₆alkyl, —O—C₁₋₆alkyl, —O—C₃₋₇cycloalkyl, —CN, and —C₀₋₁alkylene-NR^{a}R^{b}, where R^{a} and R^{b} are —C₁₋₄alkyl; and R² through R⁶ are independently selected from H, halo, —C₁₋₆alkyl, —O—C₁₋₆alkyl, —C₃₋₇cycloalkyl, —C₀₋₁alkylene-phenyl, —O—C₀₋₃alkylene-phenyl, —C(O)—C₁₋₆alkyl, and —C(O)O—C₁₋₄alkyl; and each alkyl is optionally substituted with 1 to 5 fluoro atoms.

9. The compound of claim 8, wherein each pyridine and naphthalene group is optionally substituted with one to four R¹ groups independently selected from halo, —C₁₋₆alkyl, —O—C₁₋₆alkyl, and —O—C₃₋₇cycloalkyl; R² is H, halo, —C₁₋₆alkyl, —O—C₁₋₆alkyl, —C₃₋₇cycloalkyl, —C₀₋₁alkylene-phenyl, —C(O)—C₁₋₆alkyl, or —C(O)O—C₁₋₄alkyl; R³ is H, halo, or —C₁₋₆alkyl; R⁴ is H, halo, —C₁₋₆alkyl, or —C₀₋₁alkylene-phenyl; R⁵ is H or halo; R⁶ is H, halo, or —C₁₋₆alkyl; and each alkyl is optionally substituted with 1 to 5 fluoro atoms.

10. The compound of claim 9, wherein each pyridine group is optionally substituted with one to four R¹ groups independently selected from halo, —C₁₋₆alkyl, —O—C₁₋₆alkyl, and —O—C₃₋₇cycloalkyl; R² is H, halo, —C₁₋₆alkyl, —O—C₁₋₆alkyl, —C₀₋₁alkylene-phenyl, —C(O)—C₁₋₆alkyl, or —C(O)O—C₁₋₄alkyl; R³ and R⁴ are independently H, halo, or —C₁₋₆alkyl; and R⁵ and R⁶ are independently H or halo; and each alkyl is optionally substituted with 1 to 5 fluoro atoms.

11. The compound of claim 1, wherein R^{A} is 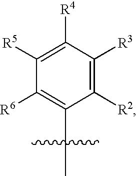
and R^{B} is selected from 2-pyridine substituted with one to two R¹ groups, 3-pyridine optionally substituted with one to two R¹ groups, 4-pyridine optionally substituted with one to two R¹ groups, thiazole, oxazole, furan, pyrazine, and pyrimidine.

12. The compound of claim 11, wherein R² is H, halo, —C₁₋₆alkyl, —O—C₁₋₆alkyl, —C₃₋₇cycloalkyl, —C₀₋₁alkylene-phenyl, —O—C₀₋₃alkylene-phenyl, —C(O)—C₁₋₆alkyl, or —C(O)O—C₁₋₄alkyl, where the alkyl in —C₁₋₆alkyl is optionally substituted with 1 to 5 fluoro atoms; R³ is H, halo, or —C₁₋₆alkyl; R⁴ is H, halo, —C₁₋₆alkyl, —O—C₁₋₆alkyl, or —C₀₋₁alkylene-phenyl; R⁵ is H or halo; and R⁶ is H, halo, or —C₁₋₆alkyl.

13. The compound of claim 11, wherein R^{B} is 3-pyridine optionally substituted with one halo group, or 4-pyridine; R² is H, halo, —C₁₋₆alkyl, —O—C₁₋₆alkyl, —C₀₋₁alkylene-phenyl, —C(O)—C₁₋₆alkyl, or —C(O)O—C₁₋₄alkyl, where the alkyl in —C₁₋₆alkyl is optionally substituted with 1 to 5 fluoro atoms; R³ is H, halo, or —C₁₋₆alkyl; R⁴ is H, halo, —C₁₋₆alkyl, or —C₀₋₁alkylene-phenyl; R⁵ is H or halo; and R⁶ is H, halo, or —C₁₋₆alkyl.

14. The compound of claim 11, wherein R^{B} is thiazole; R² is H, halo, or —C₁₋₆alkyl; R³ is H, halo, or —C₁₋₆alkyl; R⁴ is H or halo; R⁵ is H or halo; and R⁶ is H or halo.

15. The compound of claim 11, wherein R^{B} is oxazole; R² is H, halo, —C₁₋₆alkyl, —O—C₁₋₆alkyl, —C₃₋₇cycloalkyl, —C₀₋₁alkylene-phenyl, —C(O)—C₁₋₆alkyl, or —C(O)O—C₁₋₄alkyl, where the alkyl in —C₁₋₆alkyl is optionally substituted with 1 to 5 fluoro atoms; R³ is H or halo; R⁴ is H, halo, or —C₁₋₆alkyl; R⁵ is H or halo; and R⁶ is H, halo, or —C₁₋₆alkyl.

16. The compound of claim 11, wherein R^{B} is furan; R² is H; R³ is halo; R⁴ is H; R⁵ is halo; and R⁶ is H.

17. The compound of claim 11, wherein R^{B} is pyrazine; R² is H, halo, or —C₁₋₆alkyl; R³ is H, halo, or —C₁₋₆alkyl; R⁴ is H or halo; R⁵ is H or halo; and R⁶ is H or halo.

18. The compound of claim 11, wherein R^{B} is pyrimidine; R² is —C₁₋₆alkyl; R³ is H; R⁴ is halo; R⁵ is H; and R⁶ is H.

19. The compound of claim 1, wherein R^{A} is pyridine optionally substituted with one to four R¹ groups independently selected from halo, —C₁₋₆alkyl, —O—C₁₋₆alkyl, —O—C₃₋₇cycloalkyl, —CN, and —C₀₋₁alkylene-NR^{a}R^{b}, where R^{a} and R^{b} are —C₁₋₄alkyl; where each alkyl is optionally substituted with 1 to 5 fluoro atoms; and R^{B} is 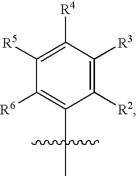
where R²-R⁶ are H.

20. The compound of claim 19, wherein R^{A} is 2-pyridine optionally substituted with one to four R¹ groups independently selected from halo, —C₁₋₆alkyl, and —O—C₁₋₆alkyl, where the alkyl in —C₁₋₆alkyl is optionally substituted with 1 to 5 fluoro atoms.

21. The compound of claim 19, wherein R^{A} is 3-pyridine is substituted with one or two R¹ groups independently selected from halo, —O—C₁₋₆alkyl, and —O—C₃₋₇cycloalkyl, where the alkyl in —O—C₁₋₆alkyl is optionally substituted with 1 to 5 fluoro atoms.

22. The compound of claim 1, wherein R^{A} is pyridine optionally substituted with one or two R¹ groups independently selected from halo and —C₁₋₆alkyl, and R^{B} is oxazole or pyridine optionally substituted with one halo group.

23. The compound of claim 22, wherein R^{A} is 5-chloro-3-methyl-2-pyridine and R^{B} is 2-oxazole, 5-fluoro-3-pyridine, or 5-clororo-3-pyridine.

24. The compound of claim 1, wherein R^{A} is naphthalene and R^{B} is oxazole or pyridine optionally substituted with one halo group.

25. The compound of claim 24, wherein R^{A} is naphthalen-1-yl or naphthalen-2-yl, and R^{B} is 2-oxazole, 3-chloro-5-pyridine, or 3-fluoro-5-pyridine.

26. The compound of claim 1, wherein R^{A} is naphthalene optionally substituted with one R¹ group, and R^{B} is 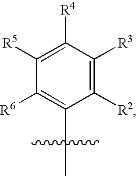
where R¹ is halo or —O—C₁₋₆alkyl; R² is H or —C₁₋₆alkyl; R³ is H, halo, —C₁₋₆alkyl, or —O—C₁₋₆alkyl; R⁴, R⁵ and R⁶ are H; and each alkyl is optionally substituted with 1 to 5 fluoro atoms.

27. The compound of claim 26, wherein R^{A} is naphthalen-1-yl; R¹ is halo or —O—C₁₋₆alkyl; R² is H or —C₁₋₆alkyl; R³ is H, halo, —C₁₋₆alkyl, or —O—C₁₋₆alkyl; and R⁴, R⁵ and R⁶ are H.

28. An intermediate useful in the synthesis of the compound of claim 1, having the formula: 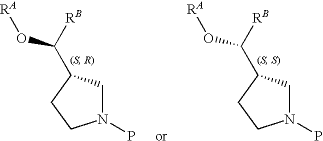
where P represents an amino-protecting group.

29. A pharmaceutical composition comprising the compound of claim 1 and a pharmaceutically acceptable carrier.

30. The pharmaceutical composition of claim 29 further comprising a second therapeutic agent selected from anti-Alzheimer's agents, anticonvulsants, antidepressants, anti-Parkinson's agents, dual serotonin-norepinephrine reuptake inhibitors, non-steroidal anti-inflammatory agents, norepinephrine reuptake inhibitors, opioid agonists, selective serotonin reuptake inhibitors, sodium channel blockers, sympatholytics, and combinations thereof.

31. A method of treating a patient that is suffering from a disease or disorder that is treated by the inhibition of the serotonin and/or the norepinephrine transporter, comprising administering a therapeutically effective amount of the compound claim 1.

32. The method of claim 31, wherein the disorder or disease is selected from pain disorders, depressive disorders, affective disorders, attention deficit hyperactivity disorders, cognitive disorders, stress urinary incontinence, chronic fatigue syndrome, obesity, and vasomotor symptoms associated with menopause.

33. The method of claim 32, wherein the pain disorder is neuropathic pain or fibromyalgia.
